SEQUENCE LISTING (136 sequences)

SEQ ID NO: 1 - protein, 23 aa, synthetic construct
  REGION 1..23: note = sequence
LQCRICGFTCRLKGNLLRHIKLH

SEQ ID NO: 2 - protein, 23 aa, synthetic construct
  REGION 1..23: note = sequence
LQCEICGFTCRRTGNLLRHIKLH

SEQ ID NO: 3 - protein, 23 aa, synthetic construct
  REGION 1..23: note = sequence
LQCEICGFTCRRKGNLMRHIKLH

SEQ ID NO: 4 - protein, 23 aa, synthetic construct
  REGION 1..23: note = sequence
LQCEICGFTCRQKMRLLRHIKLH

SEQ ID NO: 5 - protein, 23 aa, synthetic construct
  REGION 1..23: note = sequence
LQCEICGFTCRQVKNLLRHIKLH

SEQ ID NO: 6 - protein, 23 aa, synthetic construct
  REGION 1..23: note = sequence
LQCEICGFTCRQKNNLLRHIALH

SEQ ID NO: 7 - protein, 23 aa, synthetic construct
  REGION 1..23: note = sequence
LQCEICGFTCRQKNNLLRHIKRH

SEQ ID NO: 8 - protein, 23 aa, synthetic construct
  REGION 1..23: note = sequence
LQCEICGFTCRQLGNLLWHIKLH

SEQ ID NO: 9 - protein, 23 aa, synthetic construct
  REGION 1..23: note = sequence
LQCEICGFTCRRVGNLLRHIKLH

SEQ ID NO: 10 - protein, 23 aa, synthetic construct
  REGION 1..23: note = sequence
LQCWICGFTCRQKGNLLRHIYLH

SEQ ID NO: 11 - protein, 23 aa, synthetic construct
  REGION 1..23: note = sequence
LQCPICGFTCRQKSNLLRHIKLH

SEQ ID NO: 12 - protein, 23 aa, synthetic construct
  REGION 1..23: note = sequence
IQCEACGFTCRQKGNLLRHIKLH

SEQ ID NO: 13 - protein, 23 aa, synthetic construct
  REGION 1..23: note = sequence
LQCEICGFTCRQKGNMLRHIRLH

SEQ ID NO: 14 - protein, 23 aa, synthetic construct
  REGION 1..23: note = sequence
LQCEICGFTCRQKGNLIRHILLH

SEQ ID NO: 15 - protein, 23 aa, synthetic construct
  REGION 1..23: note = sequence
LQCLICGFTCRRKGNLLRHIKLH

SEQ ID NO: 16 - protein, 23 aa, synthetic construct
  REGION 1..23: note = sequence
LQCEICGFTCRRKGNLYRHIKLH

SEQ ID NO: 17 - protein, 23 aa, synthetic construct
  REGION 1..23: note = sequence
LQCIICGFTCRQKGNLLKHIKLH

SEQ ID NO: 18 - protein, 23 aa, synthetic construct
  REGION 1..23: note = sequence
LQCAICGFTSRQKGNLLRHIKLH

SEQ ID NO: 19 - protein, 23 aa, synthetic construct
  REGION 1..23: note = sequence
LQCEICGFTCRLKGNLLRHILLH

SEQ ID NO: 20 - protein, 23 aa, synthetic construct
  REGION 1..23: note = sequence
LQCKICGFTCRVKGNLLRHIKLH

SEQ ID NO: 21 - protein, 23 aa, synthetic construct
  REGION 1..23: note = sequence
LQCEICGFSCRQKGNLRRHIKLH

SEQ ID NO: 22 - protein, 23 aa, synthetic construct
  REGION 1..23: note = sequence
LQCKICGFTSRQKGNLLRHIKLH

SEQ ID NO: 23 - protein, 23 aa, synthetic construct
  REGION 1..23: note = sequence
LQCEICGFTCRKKGALLRHIKLH

SEQ ID NO: 24 - protein, 23 aa, synthetic construct
  REGION 1..23: note = sequence
LQCEICGFTCRQKHNLLRHIKNH

SEQ ID NO: 25 - protein, 23 aa, synthetic construct
  REGION 1..23: note = sequence
LQCKICGFTCRQMGNLLRHIKLH

SEQ ID NO: 26 - protein, 23 aa, synthetic construct
  REGION 1..23: note = sequence
FQCEICGFTCRQKVNLLRHIKLH

SEQ ID NO: 27 - protein, 23 aa, synthetic construct
  REGION 1..23: note = sequence
LQCEICGFTCVQKGNLLRHIALH

SEQ ID NO: 28 - protein, 23 aa, synthetic construct
  REGION 1..23: note = sequence
LQCEICGFTCRRKGNLLRHIKHH

SEQ ID NO: 29 - protein, 23 aa, synthetic construct
  REGION 1..23: note = sequence
LQCEICGFTCRQVGWLLRHIKLH

SEQ ID NO: 30 - protein, 23 aa, synthetic construct
  REGION 1..23: note = sequence
LQCMICGFTFRQKGNLLRHIKLH

SEQ ID NO: 31 - protein, 23 aa, synthetic construct
  REGION 1..23: note = sequence
LQCEICGFTCRQKTRLLRHIKLH

SEQ ID NO: 32 - protein, 23 aa, synthetic construct
  REGION 1..23: note = sequence
LQCEICGFTCRQEKNLLRHIKLH

SEQ ID NO: 33 - protein, 23 aa, synthetic construct
  REGION 1..23: note = sequence
LQCDICGFTCRQKGNLLLHIKLH

SEQ ID NO: 34 - protein, 23 aa, synthetic construct
  REGION 1..23: note = sequence
LQCEICGFTCRQKYNLLRHISLH

SEQ ID NO: 35 - protein, 23 aa, synthetic construct
  REGION 1..23: note = sequence
LQCEICGFTARRKGNLLRHIKLH

SEQ ID NO: 36 - protein, 23 aa, synthetic construct
  REGION 1..23: note = sequence
YQCVICGFTCRQKGNLLRHIKLH

SEQ ID NO: 37 - protein, 23 aa, synthetic construct
  REGION 1..23: note = sequence
FQCEICGFTCRQKHNLLRHIKLH

SEQ ID NO: 38 - protein, 23 aa, synthetic construct
  REGION 1..23: note = sequence
LQCEICGYTCRKKGNLLRHIKLH

SEQ ID NO: 39 - protein, 23 aa, synthetic construct
  REGION 1..23: note = sequence
LQCEICGFTCRQKGRLLRHIKQH

SEQ ID NO: 40 - protein, 23 aa, synthetic construct
  REGION 1..23: note = sequence
LQCEICGFTCKQKKNLLRHIKLH

SEQ ID NO: 41 - protein, 23 aa, synthetic construct
  REGION 1..23: note = sequence
LQCEVCGFTCRKKGNLLRHIKLH

SEQ ID NO: 42 - protein, 23 aa, synthetic construct
  REGION 1..23: note = sequence
LQCEICGFTCRRKGNLLRHRKLH

SEQ ID NO: 43 - protein, 23 aa, synthetic construct
  REGION 1..23: note = sequence
FQCEICGFTCRQKGRLLRHIKLH

SEQ ID NO: 44 - protein, 23 aa, synthetic construct
  REGION 1..23: note = sequence
LQCEICGFTCRQKANLKRHIKLH

SEQ ID NO: 45 - protein, 23 aa, synthetic construct
  REGION 1..23: note = sequence
LQCIICGFTCRQKGNLWRHIKLH

SEQ ID NO: 46 - protein, 23 aa, synthetic construct
  REGION 1..23: note = sequence
LQCEICGFTCRQKGNLYRHYKLH

SEQ ID NO: 47 - protein, 23 aa, synthetic construct
  REGION 1..23: note = sequence
LQCEICGFTCRQKGTLLRHIKRH

SEQ ID NO: 48 - protein, 23 aa, synthetic construct
  REGION 1..23: note = sequence
LQCEICGFTCRKKGNLLRHKKLH

SEQ ID NO: 49 - protein, 23 aa, synthetic construct
  REGION 1..23: note = sequence
LQCEICGFTCRRKGSLLRHIKLH

SEQ ID NO: 50 - protein, 23 aa, synthetic construct
  REGION 1..23: note = sequence
LQCEICGFTCRNKKNLLRHIKLH

SEQ ID NO: 51 - protein, 23 aa, synthetic construct
  REGION 1..23: note = sequence
LQCEICGFTCRQKKNLLRHISLH

SEQ ID NO: 52 - protein, 23 aa, synthetic construct
  REGION 1..23: note = sequence
FQCEICGFTCRQKRNLLRHIKLH

SEQ ID NO: 53 - protein, 23 aa, synthetic construct
  REGION 1..23: note = sequence
LQCEICGFTCRQKGNLLRHYKTH

SEQ ID NO: 54 - protein, 23 aa, synthetic construct
  REGION 1..23: note = sequence
LQCEICGFTCRQKGNLLVHIALH

SEQ ID NO: 55 - protein, 23 aa, synthetic construct
  REGION 1..23: note = sequence
LQCEICGFTCRQKFKLLRHIKLH

SEQ ID NO: 56 - protein, 23 aa, synthetic construct
  REGION 1..23: note = sequence
LQCKICGFTCRHKGNLLRHIKLH

SEQ ID NO: 57 - protein, 23 aa, synthetic construct
  REGION 1..23: note = sequence
LQCEICGFTCRQKMNLVRHIKLH

SEQ ID NO: 58 - protein, 23 aa, synthetic construct
  REGION 1..23: note = sequence
LQCRICGFTCRQKGNMLRHIKLH

SEQ ID NO: 59 - protein, 23 aa, synthetic construct
  REGION 1..23: note = sequence
LQCEICGFTCRLKGNLLLHIKLH

SEQ ID NO: 60 - protein, 23 aa, synthetic construct
  REGION 1..23: note = sequence
LQCEICGFTCRQWGNYLRHIKLH

SEQ ID NO: 61 - protein, 23 aa, synthetic construct
  REGION 1..23: note = sequence
WQCEICGFTCRQYGNLLRHIKLH

SEQ ID NO: 62 - protein, 23 aa, synthetic construct
  REGION 1..23: note = sequence
LQCEICGFTCRQPGNLLRHIKRH

SEQ ID NO: 63 - protein, 23 aa, synthetic construct
  REGION 1..23: note = sequence
LQCWICGFTCRRKGNLLRHIKLH

SEQ ID NO: 64 - protein, 23 aa, synthetic construct
  REGION 1..23: note = sequence
LQCEICGFTCRQKGNLHLHIKLH

SEQ ID NO: 65 - protein, 23 aa, synthetic construct
  REGION 1..23: note = sequence
LQCEICGFTCRQKGNLLRHIFKH

SEQ ID NO: 66 - protein, 23 aa, synthetic construct
  REGION 1..23: note = sequence
LQCVICGFTCRQKGNLVRHIKLH

SEQ ID NO: 67 - protein, 23 aa, synthetic construct
  REGION 1..23: note = sequence
LQCEICGFTCRQQLNLLRHIKLH

SEQ ID NO: 68 - protein, 23 aa, synthetic construct
  REGION 1..23: note = sequence
HQCEICGFTCRQKGRLLRHIKLH

SEQ ID NO: 69 - protein, 23 aa, synthetic construct
  REGION 1..23: note = sequence
LQCEICGFTCRQKGNLDRHIKRH

SEQ ID NO: 70 - protein, 23 aa, synthetic construct
  REGION 1..23: note = sequence
LQCWICGFTCRQKMNLLRHIKLH

SEQ ID NO: 71 - protein, 23 aa, synthetic construct
  REGION 1..23: note = sequence
LQCEICGFTCRKKNNLLRHIKLH

SEQ ID NO: 72 - protein, 23 aa, synthetic construct
  REGION 1..23: note = sequence
YQCEICGFTCRQKGNLLRHIILH

SEQ ID NO: 73 - protein, 23 aa, synthetic construct
  REGION 1..23: note = sequence
LQCHICGFTSRQKGNLLRHIKLH

SEQ ID NO: 74 - protein, 23 aa, synthetic construct
  REGION 1..23: note = sequence
WQCEICGFTCRQKGNLLRHIALH

SEQ ID NO: 75 - protein, 23 aa, synthetic construct
  REGION 1..23: note = sequence
LQCHICGFTCRQHGNLLRHIKLH

SEQ ID NO: 76 - protein, 23 aa, synthetic construct
  REGION 1..23: note = sequence
LQCQICGFTCRRKGNLLRHIKLH

SEQ ID NO: 77 - protein, 23 aa, synthetic construct
  REGION 1..23: note = sequence
LQCEICGFTCRKRGNLLRHIKLH

SEQ ID NO: 78 - protein, 23 aa, synthetic construct
  REGION 1..23: note = sequence
LQCNICGFTCRQLGNLLRHIKLH

SEQ ID NO: 79 - protein, 23 aa, synthetic construct
  REGION 1..23: note = sequence
LQCEICGFTCRKKGNLLRHIILH

SEQ ID NO: 80 - protein, 23 aa, synthetic construct
  REGION 1..23: note = sequence
LQCEICGFTCRQPGNLLRHYKLH

SEQ ID NO: 81 - protein, 23 aa, synthetic construct
  REGION 1..23: note = sequence
LQCEICGFTCRQDGRLLRHIKLH

SEQ ID NO: 82 - protein, 23 aa, synthetic construct
  REGION 1..23: note = sequence
LQCMICGFTCRQKGNLMRHIKLH

SEQ ID NO: 83 - protein, 23 aa, synthetic construct
  REGION 1..23: note = sequence
LQCWICGFTCRQKGNMLRHIKLH

SEQ ID NO: 84 - protein, 23 aa, synthetic construct
  REGION 1..23: note = sequence
LQCEICGFTCRQKGKYLRHIKLH

SEQ ID NO: 85 - protein, 23 aa, synthetic construct
  REGION 1..23: note = sequence
WQCMICGFTCRQKGNLLRHIKLH

SEQ ID NO: 86 - protein, 23 aa, synthetic construct
  REGION 1..23: note = sequence
LQCVICGFQCRQKGNLLRHIKLH

SEQ ID NO: 87 - protein, 23 aa, synthetic construct
  REGION 1..23: note = sequence
LQCEICGFTCRQKQRLLRHIKLH

SEQ ID NO: 88 - protein, 23 aa, synthetic construct
  REGION 1..23: note = sequence
LQCEICGFRCRQKGNLLRHIKKH

SEQ ID NO: 89 - protein, 23 aa, synthetic construct
  REGION 1..23: note = sequence
MQCEICGFTCRQKSNLLRHIKLH

SEQ ID NO: 90 - protein, 23 aa, synthetic construct
  REGION 1..23: note = sequence
LQCEICGFTCRQHGVLLRHIKLH

SEQ ID NO: 91 - protein, 23 aa, synthetic construct
  REGION 1..23: note = sequence
LQCWICGFTCRQKGNLWRHIKLH

SEQ ID NO: 92 - protein, 23 aa, synthetic construct
  REGION 1..23: note = sequence
LQCEICGFTCRQLRNLLRHIKLH

SEQ ID NO: 93 - protein, 23 aa, synthetic construct
  REGION 1..23: note = sequence
LQCEICGFTSRQKGRLLRHIKLH

SEQ ID NO: 94 - protein, 23 aa, synthetic construct
  REGION 1..23: note = sequence
MQCVICGFTCRQKGNLLRHIKLH

SEQ ID NO: 95 - protein, 23 aa, synthetic construct
  REGION 1..23: note = sequence
LQCLICGFTCRQKGNLLRHIRLH

SEQ ID NO: 96 - protein, 23 aa, synthetic construct
  REGION 1..23: note = sequence
LQCEICGFTCRQKLNLLRHIKRH

SEQ ID NO: 97 - protein, 23 aa, synthetic construct
  REGION 1..23: note = sequence
LQCEICGFTCRKKVNLLRHIKLH

SEQ ID NO: 98 - protein, 23 aa, synthetic construct
  REGION 1..23: note = sequence
LQCEICGFKCRQKGNLLRHIKRH

SEQ ID NO: 99 - protein, 23 aa, synthetic construct
  REGION 1..23: note = sequence
LQCEICGFTCRQKGNLLRHINNH

SEQ ID NO: 100 - protein, 23 aa, synthetic construct
  REGION 1..23: note = sequence
LQCEICGFTCRQKGRLLRHIHLH

SEQ ID NO: 101 - protein, 11 aa, synthetic construct
  REGION 1..11: note = sequence
LQCEICGFTCR

SEQ ID NO: 102 - protein, 12 aa, synthetic construct
  REGION 1..12: note = sequence
QKGNLLRHIKLH

SEQ ID NO: 103 - protein, 23 aa, synthetic construct
  REGION 1..23: note = sequence
LQCEICGFTCRQKGNLLRHIKLH

SEQ ID NO: 104 - protein, 12 aa, synthetic construct
  REGION 1..12: note = sequence
FKCHLCNYACRR

SEQ ID NO: 105 - protein, 12 aa, synthetic construct
  REGION 1..12: note = sequence
KDSVVAHKAKSH

SEQ ID NO: 106 - protein, 24 aa, synthetic construct
  REGION 1..24: note = sequence
FKCHLCNYACRRKDSVVAHKAKSH

SEQ ID NO: 107 - protein, 52 aa, synthetic construct
  REGION 1..52: note = sequence
LQCEICGFTCRQKGNLLRHIKLHSGEKPFKCHLCNYACRRKDSVVAHKAKSH

SEQ ID NO: 108 - protein, 23 aa, synthetic construct
  REGION 1..23: note = sequence
YQCEICGFTCRQKGNLLRHIKLH

SEQ ID NO: 109 - protein, 23 aa, synthetic construct
  REGION 1..23: note = sequence
FKCHLCNYACRRRDALTGHLRTH

SEQ ID NO: 110 - protein, 23 aa, synthetic construct
  REGION 1..23: note = sequence
FKCHLCNYACQRRDALTGHLRTH

SEQ ID NO: 111 - protein, 24 aa, synthetic construct
  REGION 1..24: note = sequence
FSCNICGKKFEKKDSVVAHKAKSH

SEQ ID NO: 112 - protein, 24 aa, synthetic construct
  REGION 1..24: note = sequence
FTCDRCGKRFEKLDSVKFHTLKSH

SEQ ID NO: 113 - protein, 24 aa, synthetic construct
  REGION 1..24: note = sequence
FACDQCGRRFEKAHNLNVHMSMVH

SEQ ID NO: 114 - protein, 23 aa, synthetic construct
  REGION 1..23: note = sequence
HQCPLCPFRCARKDNLKSHMKVH

SEQ ID NO: 115 - protein, 24 aa, synthetic construct
  REGION 1..24: note = sequence
FPCEFCGKRFEKPDSVAAHRSKSH

SEQ ID NO: 116 - protein, 23 aa, synthetic construct
  REGION 1..23: note = sequence
FQCNQCGASFTQKGNLLRHIKLH

SEQ ID NO: 117 - protein, 23 aa, synthetic construct
  REGION 1..23: note = sequence
FQCNQCGASFTQKGNLLRHIKLH

SEQ ID NO: 118 - protein, 23 aa, synthetic construct
  REGION 1..23: note = sequence
LQCEICGFTCRQKASLNWHMKKH

SEQ ID NO: 119 - protein, 23 aa, synthetic construct
  REGION 1..23: note = sequence
FACVICGRKFRNRGLMQKHLKNH

SEQ ID NO: 120 - protein, 23 aa, synthetic construct
  REGION 1..23: note = sequence
FVCPRCGRGFSQPKSLARHLRLH

SEQ ID NO: 121 - protein, 23 aa, synthetic construct
  REGION 1..23: note = sequence
LQCEICGYQCRQRASLNWHMKKH

SEQ ID NO: 122 - protein, 23 aa, synthetic construct
  REGION 1..23: note = sequence
LQCEVCGFQCRQRASLKYHMTKH

SEQ ID NO: 123 - protein, 23 aa, synthetic construct
  REGION 1..23: note = sequence
LQCEICGFTCRQKASLNWHQRKH

SEQ ID NO: 124 - protein, 23 aa, synthetic construct
  REGION 1..23: note = sequence
YQCKVCGRAFKRVSHLTVHYRIH

SEQ ID NO: 125 - protein, 23 aa, synthetic construct
  REGION 1..23: note = sequence
YRCRACGRACSRLSTLIQHQKVH

SEQ ID NO: 126 - protein, 23 aa, synthetic construct
  REGION 1..23: note = sequence
HECKLCGASFRTKGSLIRHHRRH

SEQ ID NO: 127 - protein, 23 aa, synthetic construct
  REGION 1..23: note = sequence
FQCPICGLVIKRKSYWKRHMVIH

SEQ ID NO: 128 - protein, 23 aa, synthetic construct
  REGION 1..23: note = sequence
FKCHLCNYACRRRDALTGHLRTH

SEQ ID NO: 129 - protein, 23 aa, synthetic construct
  REGION 1..23: note = sequence
FKCHLCNYACQRRDALTGHLRTH

SEQ ID NO: 130 - protein, 24 aa, synthetic construct
  REGION 1..24: note = sequence
FSCNICGKKFEKKDSVVAHKAKSH

SEQ ID NO: 131 - protein, 24 aa, synthetic construct
  REGION 1..24: note = sequence
FTCDRCGKRFEKLDSVKFHTLKSH

SEQ ID NO: 132 - protein, 24 aa, synthetic construct
  REGION 1..24: note = sequence
FACDQCGRRFEKAHNLNVHMSMVH

SEQ ID NO: 133 - protein, 23 aa, synthetic construct
  REGION 1..23: note = sequence
HQCPLCPFRCARKDNLKSHMKVH

SEQ ID NO: 134 - protein, 24 aa, synthetic construct
  REGION 1..24: note = sequence
FPCEFCGKRFEKPDSVAAHRSKSH

SEQ ID NO: 135 - protein, 23 aa, synthetic construct
LQCEICGFTCRRVNNLLRHIALH

SEQ ID NO: 136 - protein, 52 aa, synthetic construct
LQCEICGFTCRRVNNLLRHIALHSGEKPFKCHLCNYACRRKDSVVAHKAKSH

(19) United States
(12) Patent Application Publication
(10) Pub. No.: US 2025/0206789 A1
(43) Pub. Date: Jun. 26, 2025

(54) ZINC FINGER DEGRON SEQUENCES

(71) Applicant: STICHTING HET NEDERLANDS KANKER INSTITUUT-ANTONI VAN LEEUWENHOEK ZIEKENHUIS, AMSTERDAM (NL)

(72) Inventors: Ali Can SAHILLIOGLU, AMSTERDAM (NL); Antonius Nicolaas Maria SCHUMACHER, AMSTERDAM (NL)

(21) Appl. No.: 18/845,540

(22) PCT Filed: Mar. 17, 2023
(86) PCT No.: PCT/NL2023/050136

§ 371 Date: Sep. 10, 2024

(30) Foreign Application Priority Data
Mar. 18, 2022 (NL) 2031325

(51) Int. Cl. C07K 14/47

(52) U.S. Cl. CPC C07K 14/4703; C07K 2319/81; C07K 2319/95

(57) ABSTRACT

This invention pertains in general to novel zinc finger degron sequences wherein the zinc finger degron comprises at least one non-natural zinc finger domain in the form of a hybrid composed of a first portion from a first zinc finger and a second portion form a second zinc finger, preferably wherein the first portion comprises a beta-hairpin of a first zinc finger and the second portion comprises an alpha-helix of a second zinc finger. In a preferred embodiment the degron tag comprises a further, second, non-natural hybrid zinc finger domain.

### FIELD OF THE INVENTION

This invention pertains in general to novel zinc finger degron sequences wherein the zinc finger degron comprises at least one non-natural zinc finger domain in the form of a hybrid composed of a first portion from a first zinc finger and a second portion form a second zinc finger, preferably wherein the first portion comprises a beta-hairpin of a first zinc finger and the second portion comprises an alpha-helix of a second zinc finger. In a preferred embodiment the degron tag comprises a further, second, non-natural hybrid zinc finger domain. In a preferred embodiment the zinc finger domain comprises at least two amino acids substitutions. Also provided is for fusion proteins comprising a degron tag according to the invention and for use of the degron tags according to the invention in methods that involve controlling protein-of-interest levels, cellular activity, expression and the like.

### BACKGROUND OF THE INVENTION

The background description includes information that may be useful in understanding the present invention. It is not an admission that any of the information provided herein is prior art or relevant to the presently claimed invention, or that any publication specifically or implicitly referenced is prior art.

Cell behavior is too a large extent controlled by the expression levels of various protein including key regulator proteins. To achieve control over such cell behavior, synthetic or artificial protein stability control tools that allow reversible and titratable expression of a protein of interest (POI), for example on a timescale of minutes, by exogenously provided factors, for example (small) compounds, have been developed.

In particular, strategies to control POI proteolysis rate (i.e., the rate of protein degradation, for example intracellular protein degradation) in response to exogenously provided factors such as membrane permeable small molecules have been developed. Well-known examples include the SMASh/Asunaprevir (Chung et al. Nat Chem Biol. 2015 September; 11 (9): 713-20), FKBP12F36V/dTAG-13 (Nabet et al. Nat Chem Biol. 2018 May; 14 (5): 431-441), zinc finger degron/IMID (Sievers et al. Science. 2018 Nov. 2; 362 (6414): eaat0572), and DD/Shield1 (Haugwitz et al. Biotechniques. 2008 March; 44 (3): 432-3) platforms.

These platforms and the principals on which these platforms are based are well-known to the person skilled in the art and have been extensively described in both scientific and patent literature (see, for example, for zinc finger degron/IMID WO2019007869, WO2020132039, WO2021188286, or WO2021080427).

Most of these platforms and the methods used therein involve fusion of the POI with a small molecule-dependent degron domain (i.e., a domain or portion of a protein that is important in regulation of protein degradation rates), and preferential interaction of the degron (domain) with the proteasomal complex in the presence of a small molecule regulator leads to subsequent degradation of the POI-degron fusion protein.

Alternatively, a POI can be fused with a degron domain that is inherently unstable and hence prone to proteolysis, and stabilization of the folded protein state by small molecule addition is used to prevent such proteolysis.

A POI can also be fused with a regulation domain that contains a constitutively active degron domain, a protease and the corresponding protease cleavage site in the linker between POI and degron tag. In the absence of protease inhibitor, the degron domain is proteolytically removed from the POI, thereby, for example, stabilizing POI expression. In contrast, in the presence of protease inhibitor, degradation of the fusion protein is induced.

In spite of the major interest in the development of protein stability control systems that can be used in cell therapy applications, a number of challenges remains ahead. For example, a preferred protein stability regulator that is used in clinical cell therapy products should be composed of human sequences in order to minimize the risk of immune-mediated rejection, and the immunogenicity of e.g., the SMASh-tag degron system that is based on the hepatitis C virus (HCV) derived NS3/4A protease (Tan et al, PLOS One. 2017; 12 (7): e0181578) is likely to limit its clinical utility.

In addition, the small molecule regulators of protein stability control domains should be safe and preferably be clinically approved. The absence of clinical approval of the dTAG-13 PROTAC that is used in the FKBP12F36V-tag degron system complicates the clinical development of FKBP12F36V-based regulation systems. In addition, the large size of PROTAC molecules is considered as a challenge for administration and metabolism of this drug class (Hu et al. Chembiochem. 2021 Sep. 8. doi: 10.1002/cbic.202100270. Online ahead of print).

Furthermore, and importantly, the small molecule concentration that is sufficient to induce biologically meaningful control over protein levels, for example meaningful control over protein degradation (e.g., degradation of the POI fused to the degron) should preferably be compatible with clinical use, and in particular in the context of long-term small molecule administration, the side effects of such administration may form a concern.

The immunomodulatory imide drug (IMiD) inducible zinc finger degron system described by Sievers et al. Science. 2018 Nov. 2; 362 (6414): eaat0572, employs fusion of a POI with a short zinc finger degron tag (sometimes also referred to as zinc finger domain and/or zinc finger polypeptide) that facilitates recruitment of POI-zinc finger fusion degron protein to the IMID/CRBN E3 ligase complex, e.g., in the presence of the small molecules known as IMID. It is generally thought that IMiDs bind to Cereblon (CRBN), the substrate receptor of the CRL4CRBN E3 ubiquitin ligase and that CRBN can recruit (fusion) proteins containing a zinc finger degron tag through interaction with the zinc finger degron, which interaction is mediated by the IMiDs such as thalidomide and its derivatives. This zinc finger degron system is based on human protein sequences, thus limiting the risk of immune-mediated rejection. In addition, protein stability is regulated by clinically approved small molecules, such as thalidomide, pomalidomide and lenalidomide (as examples of IMiDs), thereby facilitating clinical development of these systems.

In preclinical work, this protein stability control system has demonstrated its value in clinically relevant applications, such as the regulation of CAR-T cell activity, when such zinc finger degrons are fused to CARs (Jan et al. Sci Transl Med. 2021 Jan. 6; 13 (575): eabb6295). Furthermore, we recently reported the use of the IMiD/zinc finger system in the Chemically Regulated and SH2-delivered—Inhibitory Tail (CRASH-IT) switch platform that allows control over cellular activity levels of a variety of cell therapy platforms, such as CAR-T, TCR-T, and NK cells (Sahillioglu et al., WO2021080427).

While the fact that zinc finger degrons are composed of human sequence and can be controlled using approved molecules facilitates clinical application of this system, the clinical use of these IMID molecules in the treatment of patients with hematological cancers is associated with substantial side effects at therapeutically effective concentrations. As an example, in a study evaluating high (25 mg per day) and low (5 mg per day) dose lenalidomide maintenance therapy in multiple myeloma patients, lenalidomide dose was shown to be correlated with both toxicity and efficacy, and dose reductions due to toxicity, in particular neutropenia, were frequent in the high-dose lenalidomide cohort. (Fenk et al. Clin Cancer Res. 2020 Nov. 15; 26 (22): 5879-5886)./pct

Because of the concerns associated with IMID use at higher doses, the identification of novel zinc finger degron sequences that can be regulated at lower drug doses is attractive.

In light of this, new products, compositions, methods and uses for controlling (expression) levels of proteins-of-interest using improved and novel zinc finger degrons sequences would be highly desirable but are not yet readily available. In particular, there is a clear need in the art for reliable, efficient and reproducible products, compositions, methods and uses that allow, for example, regulating (expression) levels of proteins-of-interest using novel zinc finger degron sequences (zinc finger degron tags, domains, polypeptides) in treatment or prevention of disease in a manner that is compatible with clinical use, for example allowing to employ reduced concentrations of small molecules (lower drug doses), in particular IMiDs, that regulate protein degradation/stability by interacting with the degron sequences, for example, fused with the protein-of-interest. Accordingly, the technical problem underlying the present invention can been seen in the provision of such products, compositions, methods and uses for complying with any of the aforementioned needs. The technical problem is solved by the embodiments characterized in the claims and herein below.

### SUMMARY OF THE INVENTION

The scope of the invention is defined by the claims. Subject-matter which is not encompassed by the scope of the claims does not form part of the present claimed invention.

### BRIEF DESCRIPTION OF THE DRAWINGS

Embodiments of the invention are further described hereinafter with reference to the accompanying drawings, in which:

FIG. 1: Sensitivity of zinc finger degrons to IMiD can be improved by alteration of the second zinc finger sequence. (A) Schematic representation of a panel of zinc finger degrons containing the ZFP91 ZF4 beta hairpin and IKZF1 ZF2 alpha helix as a hybrid first zinc finger. In this panel, the degrons contain second zinc finger sequences selected from IKZF1 ZF3 (single hybrid, dual zinc finger degron), IKZF1 ZF3 beta hairpin-ZFP91 ZF5 alpha helix (double hybrid degron), or the degron does not contain a second zinc finger (single hybrid, single zinc finger degron). (B-E) Primary human T cells modified with the HLA class I restricted CDK4 TCR plus a Zap70-PD1-degron CRASH-IT switch, in which the degron sequence is selected from (A), were pretreated with indicated concentrations of thalidomide. Data depict intracellular IFNγ, IL2, TNFα, and cell surface LAMP1 expression of CDK4 TCR+, EGFP high, CD8+ T cells upon co-culture with NKIRTIL006 melanoma cells in the continued presence of indicated concentrations of thalidomide. Error bars represent standard deviation (n=2). Data are representative of two independent experiments.

FIG. 2: Schematic overview of non-limiting embodiments according to the invention.

FIG. 3: Design of the synthetic zinc finger (SynFinger) library. The SynFinger library contains Zap70-PD1-degron CRASH-IT switch variants, in which the parental zinc finger degron contains single or dual amino acid substitutions, or no substitutions. The figure depicts the sequence of the double hybrid degron that is used as the parental zinc finger sequence in the SynFinger library screen. Amino acids indicated with arrows are substituted to any of the other genetically encoded amino acids except cysteine. 2 cysteines and a conserved glycine in the ZFP91 ZF4 beta hairpin, as well as 2 histidines in the IKZF1 ZF2 alpha helix, were kept constant. Substitution mutations are numbered starting with the first amino acid of the ZFP91 ZF4beta hairpin (marked with asterisk).

FIG. 4: Enrichment of certain amino acid substitutions in the top 100 degrons. SynFinger degrons were ranked according to enrichment index (EI) values following removal of SynFingers with low sequence reads (see Examples). The graph depicts the number of times each amino substitution was found in combination with other amino acid substitutions in the top 100 SynFinger degrons. For example, the Q12R mutation was observed in 11 dual amino acid substitution combinations, together with either K13T, L17M, K13V, E4L, L17Y, L22H, C10A, 120R, N15S, E4W and E4Q, amongst the top 100 SynFingers.

FIG. 5: SynFingers fusion proteins enable sensitive control of immune cell functions. A subset of SynFingers identified in the SynFinger library screen were individually validated. (A-H) Primary human T cells were modified with the HLA class I restricted CDK4 TCR plus a Zap70-Siglec11-degron CRASH-IT switch, in which the degron sequence contains the parental zinc finger degron (double hybrid degron depicted in FIGS. 1 and 3), or the parental zinc finger degron with the following mutations: G14N/K21A, G14M/N15R, L17I/K21L, E4R/Q12L, Q12R/K13T, or Q12R/K13V. As a control, primary human T cells modified with the HLA class I restricted CDK4 TCR plus vector control were used. Cells were pretreated with 5 nM lenalidomide or left untreated. Data depict intracellular IFNγ, IL2, TNFα, and cell surface LAMP1 expression of CDK4 TCR+, EGFP intermediate, CD8+ T cells upon co-culture with NKIRTIL006 melanoma cells in the continued absence (E-H) or continued presence of 5 nM lenalidomide (A-D). Dashed horizontal lines indicate cytokine production and degranulation levels of cells modified with a CRASH-IT switch containing the parental zinc finger degron. Error bars represent standard deviation (n=2). SynFinger and vector control groups were compared to the parental zinc finger group using a one-sided t test: *=p<0.05, **=p<0.01, ***=p<0.001, ****=p<0.0001, n.s.=not significant. Data are representative of two independent experiments.

FIG. 6: SynFinger containing CRASH-IT switches enable restoration of cytokine production and degranulation at lower IMID concentrations as compared to zinc finger degrons not according to the invention. (A-D) Primary human T cells modified with the HLA class I restricted CDK4 TCR plus a Zap70-Siglec11-degron CRASH-IT switch, in which the degron sequence contains prior art zinc finger degrons (single hybrid, dual zinc finger degron depicted in FIG. 1), the parental zinc finger degron (double hybrid degron depicted in FIGS. 1 and 3), or the parental zinc finger degron with the following mutations: G14N/K21A, L17I/K21L, or Q12R/K13V, were pretreated with the indicated concentrations of lenalidomide or left untreated. Data depict intracellular IFNγ, IL2, TNFα, and cell surface LAMP1 expression of CDK4 TCR+, EGFP intermediate, CD8+ T cells upon co-culture with NKIRTIL006 melanoma cells in the continued presence or absence of the indicated concentration of lenalidomide (A-D). Error bars represent standard deviation (n=3). Data are representative of two independent experiments. (E) Table depicts EC50Lenalidomide (nM) values of cells in (A-D), and EC50 fold change between the prior art zinc finger degron and a SynFinger containing the Q12R/K13V substitutions.

FIG. 7: Combination of dual substitution sets can further increase lenalidomide sensitivity of SynFinger degron. (A-D) Primary human T cells modified with the HLA class I restricted CDK4 TCR plus a Zap70-Siglec11-degron CRASH-IT switch, in which the degron sequence contains the parental zinc finger degron (double hybrid degron depicted in FIGS. 1 and 3), or the parental zinc finger degron with the following mutations: G14N/K21A, Q12R/K13V, or Q12R/K13V/G14N/K21A, were pretreated with the indicated concentrations of lenalidomide or left untreated. Data depict intracellular IFNγ, IL2, TNFα, and cell surface LAMP1 expression of CDK4 TCR+, EGFP intermediate, CD8+ T cells upon co-culture with NKIRTIL006 melanoma cells in the continued presence or absence of the indicated concentration of lenalidomide (A-D). Error bars represent standard deviation (n=3). Data are representative of two independent experiments.

### DEFINITIONS

A portion of this disclosure contains material that is subject to copyright protection (such as, but not limited to, diagrams, device photographs, or any other aspects of this submission for which copyright protection is or may be available in any jurisdiction.). The copyright owner has no objection to the facsimile reproduction by anyone of the patent document or patent disclosure, as it appears in the Patent Office patent file or records, but otherwise reserves all copyright rights whatsoever.

Various terms relating to the methods, compositions, uses and other aspects of the present invention are used throughout the specification and claims. Such terms are to be given their ordinary meaning in the art to which the invention pertains, unless otherwise indicated. Other specifically defined terms are to be construed in a manner consistent with the definition provided herein. Although any methods and materials similar or equivalent to those described herein can be used in the practice for testing of the present invention, the preferred materials and methods are described herein. For purposes of the present invention, the following terms are defined below.

As used herein, the singular form terms “a,” “an,” and “the” include plural referents unless the content clearly dictates otherwise. Thus, for example, reference to “a degron tag” includes a combination of two or degron tags, and the like. For example, a method for administrating a drug or a fusion protein includes the administrating of a plurality of the molecules (e.g., 10's, 100's, 1000's, 10's of thousands, 100's of thousands, millions, or more molecules).

As used herein, “about” and “approximately”, when referring to a measurable value such as an amount, a temporal duration, and the like, is meant to encompass variations of ±20% or ±10%, more preferably ±5%, even more preferably ±1%, and still more preferably ±0.1% from the specified value, as such variations are appropriate to perform the disclosed invention.

As used herein, “and/or” refers to a situation wherein one or more of the stated cases may occur, alone or in combination with at least one of the stated cases, up to with all of the stated cases.

As used herein, “at least” a particular value means that particular value or more. For example, “at least 2” is understood to be the same as “2 or more” i.e., 2, 3, 4, 5, 6, 7, 8, 9, 10, 11, 12, 13, 14, 15, . . . , etc. As used herein, the term “at most” a particular value means that particular value or less. For example, “at most 5” is understood to be the same as “5 or less” i.e., 5, 4, 3, . . . 10, −11, etc.

As used herein, “comprising” or “to comprise” is construed as being inclusive and open ended, and not exclusive. Specifically, the term and variations thereof mean the specified features, steps or components are included. These terms are not to be interpreted to exclude the presence of other features, steps or components. It also encompasses the more limiting “to consist of”.

As used herein, “conventional techniques” or “methods known to the skilled person” refer to a situation wherein the methods of carrying out the conventional techniques used in methods of the invention will be evident to the skilled worker. The practice of conventional techniques in molecular biology, biochemistry, cell culture, genomics, sequencing, medical treatment, pharmacology, immunology and related fields are well-known to those of skill in the art and are discussed, in various handbooks and literature references.

As used herein, “exemplary” or “for example” means “serving as an example, instance, or illustration,” and should not be construed as excluding other configurations, including those disclosed herein.

Where in the context of the current invention differences exist between the sequences disclosed in the description and those in the sequence listing, the sequence indicated in the description prevails except for where it is clear something else is intended. Wherein reference is made to proteins, or portion thereof, in a preferred embodiment, these relate to human proteins, or portions thereof.

Throughout this disclosure, various aspects of the invention can be presented in a range format. It should be understood that the description in range format is merely for convenience and should not be construed as a limitation on the scope of the invention. The description of a range should be considered to have specifically disclosed all the possible subranges as well as individual numerical values within that range including both integers and non-integers. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 etc. This applies regardless of the breadth of the range.

As used herein the term “fusion protein” refer to any polypeptide which is not normally found in nature is a species, in particular a polypeptide in which one or more part of the amino acids sequence are not associated with each other in nature. For example, a fusion protein may comprise a N-terminal part consisting of a first sequence of amino acids and a C-terminal part consisting of a second sequence of amino acids that are not associated with each other in nature and/or are not associated with each other in nature in this order. A fusion protein may for example be obtained from transcription and translation of a fusion gene of nucleic acid. Such fusion gene may be created by joining parts of two different genes/nucleic acid sequences. Within the context of the current invention the fusion protein may, for example, comprise a degron tag according to the invention, or a hybrid zinc finger polypeptide according to the invention, fused to a protein of interest.

As used herein, the term “host cell” refers to a cell into which exogenous nucleic acid (polynucleotides) and/or exogenous polypeptide has been introduced, including the progeny of such cells. Host cells may include “transformants” and “transformed cells,” which include the primary transformed cell and progeny derived therefrom without regard to the number of passages. Progeny may not be completely identical in nucleic acid content to a parent cell but may contain mutations. Mutant progeny that has the same function or biological activity as screened or selected for in the originally transformed cell are included herein. Host cells include in vitro host cells and in vivo host cells.

As used herein “immunomodulatory drugs”, “Immunomodulatory imide drugs” or “IMiDs” refers to compounds known in the art. IMiDs include thalidomide, pomalidomide, lenalidomide, iberdomide (CC-220), avadomide (CC-122), and CC-885, or pharmaceutically acceptable salts thereof; these compounds may also be referred to as cereblon modulators (CRBN modulators). Thalidomide, lenalidomide, and pomalidomide have each been approved for treatment of various diseases while other IMiDs or cereblon modulators are under review. The compounds may be in the form of a free acid or free base, or a pharmaceutically acceptable salt.

As used herein, “in vivo” refers to an event that takes place in a subject's body; “in vitro” refers to an event that takes places outside of a subject's body. For example, an in vitro assay or method encompasses any assay or method conducted outside of a subject. In vitro assays or methods encompass cell-based assays in which cells, alive or dead, are employed. In vitro assays also encompass a cell-free assay in which no intact cells are employed.

As used herein, the term “isolated” when referring to a polynucleotide (nuclei acid) or polypeptide (protein), refers to proteins or nucleic acids being present in a non-naturally occurring environment, e.g. are separated from their naturally occurring environment. For example, an isolated protein or polypeptide according to the invention relates to a protein which is no longer in its natural environment, for example, in vitro or in a recombinant host cell. The terms, next to being isolated from naturally occurring source, also refers to such protein or nucleic acid being artificially or synthetically produced. Within the context of the current invention, it will be clear for the skilled person if a reference to a protein, polypeptide, nucleic acid, or polynucleotide includes reference to an “isolated” protein, polypeptide, nucleic acid, or polynucleotide.

As used herein, the term “linker” as used in reference to a part of a protein refers to a stretch of amino acids which joins two part of the proteins together, for example in a fusion protein. Generally, such molecules have no specific biological activity other than to join or to preserve some minimum distance or other spatial relationship between the proteins. However, in certain embodiments, the linker may be selected to influence some property of the linker and/or the protein such as the folding, net charge, or hydrophobicity of the linker.

As used herein, the term “non-natural” refers to a polypeptide, polynucleotide, or domain comprised in such polypeptide or polynucleotide and that is not known to exist in nature, for example, that is not known to exist in (human) cells, i.e., wherein one or more parts of the polypeptide, polynucleotide or domain are not associated with each other in nature. One example being a fusion protein as defined herein. As explained herein, in particular the term “non-natural” refers to the fact the first hybrid zinc finger domain is comprised of a first portion and a second portion and wherein the first portion is obtained from a first Cys₂-His₂ zinc finger domain and the second portion is obtained form a second Cys₂-His₂ zinc finger domain, and wherein the first and second Cys₂-His₂ zinc finger domain are different from each other. The non-natural first hybrid zinc finger domain is thus a fusion protein comprised of said first portion and said second portion. Alternatively the non-natural first hybrid zinc finger domain may thus be referred to as first hybrid zinc finger domain. As the skilled person will understand, the same analogy is applicable to the term “non-natural second hybrid zinc finger domain”, which therefore may also be referred to as “second hybrid zinc finger domain”.

As used herein the term “nucleic acid” or “polynucleotide” refers to any polymers or oligomers of (contiguous) nucleotides. The nucleic acid may be DNA or RNA, or a mixture thereof, and may exist permanently or transitionally in single-stranded or double-stranded form, including homoduplex, heteroduplex, and hybrid states.

As used herein, the term “pharmaceutical composition” refers to a composition formulated in pharmaceutically acceptable or physiologically acceptable compositions for administration to a cell or subject. The compositions of the invention may be administered in combination with other agents as well, provided that the additional agents do not adversely affect the ability of the composition to deliver the intended therapy. The pharmaceutical composition often comprises, in addition to a pharmaceutical active agent, one or more pharmaceutical acceptable carriers (or excipients). The pharmaceutical compositions be specially formulated for administration in solid or liquid form, including those adapted for the following: (1) oral administration, for example, drenches (aqueous or non-aqueous solutions or suspensions), tablets, boluses, powders, granules, pastes; (2) parenteral administration, for example, by subcutaneous, intramuscular or intravenous injection as, for example, a sterile solution or suspension; (3) topical application, for example, as a cream, ointment or spray applied to the skin; (4) intravaginally or intrarectally, for example, as a pessary, cream or foam; or (5) aerosol, for example, as an aqueous aerosol, liposomal preparation or solid particles containing the compound. Drugs, therapeutic agents, medicaments and pharmaceutical compositions according to the present invention may be formulated for administration by a number of routes, including but not limited to, parenteral, intravenous, intra-arterial, intramuscular, intratumoral and oral. Drugs, therapeutic agents, medicaments and compositions may be formulated in fluid or solid form. Fluid formulations may be formulated for administration by injection to a selected region of the human or animal body.

As used herein, “protein” or “polypeptide” are used interchangeably and refer to molecules consisting of a chain of amino acids, without reference to a specific mode of action, size, 3-dimensional structure or origin. A “fragment” or “portion” or “domain” of a protein may thus still be referred to as a “protein.” A protein as defined herein and as used in any method as defined herein may be an isolated protein, a natural protein or a non-natural protein. An “isolated protein” is used to refer to a protein which is no longer in its natural environment, for example in vitro or in a recombinant bacterial or plant host cell. In particular embodiments of the invention the protein is a protein that is involved in regulating cell behavior, in particular that can regulate cell behavior in the context of the treatment of a disease. In other words, the terms “portion” or “domains” refer to amino acid sequences that are less than the full protein sequence of any protein mentioned herein. The term “domains” is also more specifically used herein to refer to functional domains known in the art, e.g., zinc-finger domains, extracellular domains, intracellular domains, signaling domains, intracellular signaling domains, cytoplasmic domains and transmembrane domains.

As used herein, “sequence” or “nucleotide sequence” may refer to the order of nucleotides of, or within a nucleic acid. In other words, any order of nucleotides in a nucleic acid may be referred to as a sequence or nucleotide sequence. The term “amino acid sequence” refers to the order of amino acids of, or within a polypeptide (or protein). In other words, any order of amino acids in a polypeptide may be referred to as a sequence or amino acid sequence.

As used herein, a “subject” is to indicate the organism to be treated e.g., to which administration is contemplated. The subject may be any subject in accordance with the present invention, including, but not limited to humans, males, females, infants, children, adolescents, adults, young adults, middle-aged adults or senior adults and/or other primates or mammals. Preferably the subject is a human patient. A subject may have been diagnosed with a cancer or be suspected of having a cancer.

As used herein, “treatment”, “treating”, “palliating”, “alleviating” and “ameliorating” in the context of a subject to be treated, all refer to an approach for obtaining beneficial or desired results including, but not limited to, therapeutic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder.

As used herein, the terms “construct”, “nucleic acid construct”, “vector”, and “expression vector” may be used interchangeably and are defined as a man-made nucleic acid molecule resulting from the use of recombinant DNA technology. These constructs and vectors therefore do not include naturally occurring nucleic acid molecules although a nucleic acid construct may comprise (parts of) naturally occurring nucleic acid molecules.

### DETAILED DESCRIPTION

The invention is defined herein, and in particular in the accompanying claims.

It is contemplated that any method, use or composition described herein can be implemented with respect to any other method, use or composition described herein. Embodiments discussed in the context of methods, use and/or compositions of the invention may be employed with respect to any other method, use or composition described herein. Thus, an embodiment pertaining to one method, use or composition may be applied to other methods, uses and compositions of the invention as well.

Any references in the description to methods of treatment also refer to the compounds, pharmaceutical compositions and medicaments of the present invention for use in a method for treatment of the human (or animal) body by therapy.

The present invention provides compositions that include a degron tag and/or hybrid zinc finger polypeptide, and methods for modulating protein abundance in a target-specific manner via the degron tags and/or hybrid zinc finger polypeptide.

The invention may target endogenous and exogenous (e.g., therapeutic) proteins. As disclosed herein, degron tags and/or hybrid zinc finger polypeptides are peptides that when fused to a target protein of interest (POI), may transform the POI into a substrate for cereblon (CRBN)-dependent ubiquitination and degradation, which is induced by the administration of immunomodulatory drugs (IMiDs). Without intending to be bound by any theory, it is believed that IMiDs bind cereblon forming a complex that has binding specificity for the degron tags and/or hybrid zinc finger polypeptides. Consequently, fusion proteins with a POI become substrates for cereblon-dependent ubiquitination and degradation. Therefore, the degron tags and/or hybrid zinc finger polypeptides of the present invention may be useful for targeted degradation of POIs.

As embodied and broadly described herein, the present invention is directed to the surprising finding that zinc finger degrons and/or zinc finger polypeptides can be provided and that display improved sensitivity towards small molecules, in particular towards IMiDs, such as those disclosed herein, that regulate protein degradation/stability by interacting with these degron and/or polypeptide sequences.

The zinc finger degrons and/or zinc finger polypeptides according to the invention thus allow using reduced concentrations of these small molecules, in particular IMiDs, to control protein degradation, expression and/or stability of a protein of interest (and therewith, for example, cellular activity (total activity in a cell) of the protein of interest; for example, fused to the degron tags, zinc finger degrons and/or zinc finger polypeptides-according to the invention).

The zinc finger degrons (degron tag) and/or zinc finger polypeptides according to the invention may therefore provide for better control of protein degradation, expression and/or stability of a protein of interest (for example as comprised in a fusion protein comprising the protein of interest and the zinc finger degron or zinc finger polypeptide according to the invention) at similar or reduced concentrations of the small molecules, in particular IMiDs. At the same time, it may allow for more sensitive regulation of the degradation, expression, level and/or stability of the POI.

In other words, and in an embodiment of the invention, the zinc finger degrons (degron tag) and/or zinc finger polypeptides according to the invention allow for control of the level (expression) of a protein of interest, for example expressed in a cell or in a subject, by regulating degradation and/or stability thereof at reduced concentration of the small molecules, in particular IMiDs, that regulate protein degradation/stability by interacting with these degron and/or polypeptide sequences.

As will be apparent from the current disclosure it has, for example, surprisingly be found that improved zinc finger degrons and/or zinc finger polypeptides according to the invention may obtained by providing for degron tags and/or polypeptides that comprise a first and a second zinc finger domain, wherein each zinc finger domain generally comprises of a beta hairpin (within the context of the current invention also sometimes referred to as a “first” or “third” portion) obtained from a first Cys2-His2 zinc finger domain and an alpha helix from a second Cys2-His2 zinc finger domain (within the context of the current invention also sometimes referred to as a “second” or “fourth” portion).

In particular it was found that the first zinc finger domain is a hybrid zinc finger domain, i.e., the first zinc finger domain is comprised of a first portion and a second portion that together do not exist in nature (non-natural). For example, the first zinc finger domain is a non-natural hybrid zinc finger domain obtained by combining a beta hairpin from a first Cys2-His2 zinc finger domain and an alpha helix from a second Cys2-His2 zinc finger domain, and wherein the first and second Cys2-His2 zinc finger domain are not identical or the same.

With respect to the second zinc finger domain, it was surprisingly found that, preferably, also the second zinc finger domain comprised in the zinc finger degrons and/or zinc finger polypeptides according to the invention is a hybrid zinc finger domain, like as described for the first hybrid zinc finger domain above. The second hybrid zinc finger domain may be the same as or different from the first hybrid zinc finger domain. Preferably, the second hybrid zinc finger domain is different from the first hybrid zinc finger domain.

At the same time it was surprisingly found that when the first hybrid zinc finger domain comprised in the zinc finger degron of the invention and/or the first hybrid zinc finger domain comprised in a zinc finger polypeptide according to the invention, further comprises at least two amino acid substitutions, as disclosed herein in detail, sensitivity of the zinc finger degron (degron tag) of the invention and/or the zinc finger polypeptide of the invention to small molecules, in particular IMiDs, that regulate protein degradation/stability by interacting with these degron and/or polypeptide sequences, is even further improved.

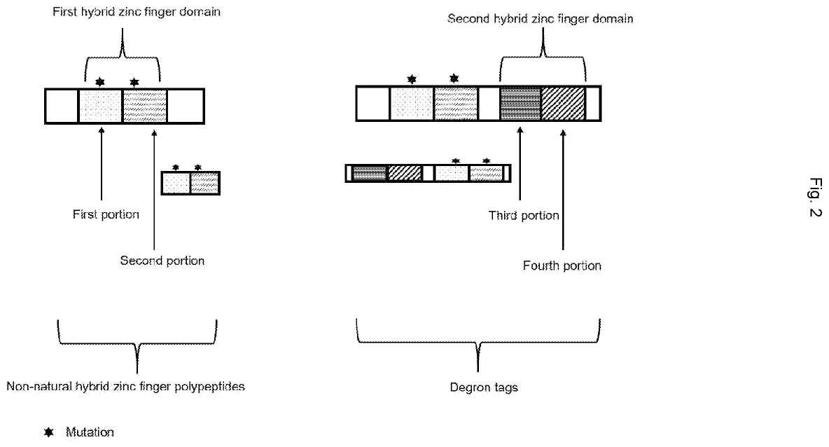

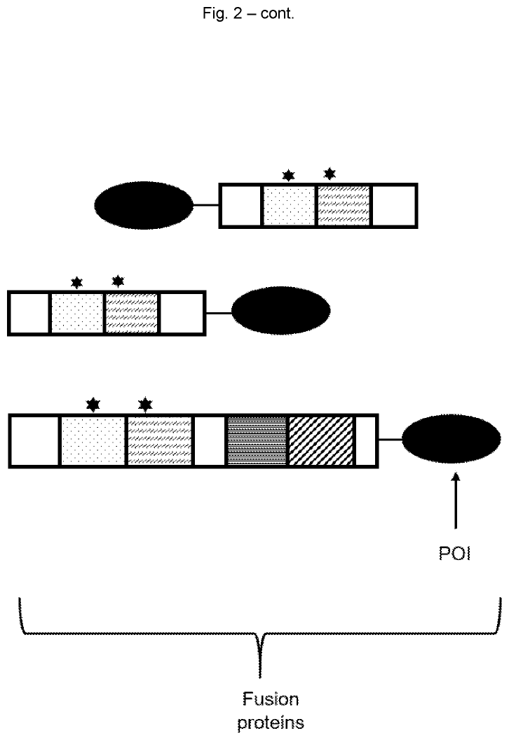

In summary, it was surprisingly found that improved zinc finger domains and degrons (e.g., degron tags) can be provided by providing a non-natural hybrid zinc finger polypeptide comprising a first hybrid zinc finger domain (as defined herein) and comprising at least two amino acid substitutions. In a preferred embodiment wherein said hybrid zinc finger polypeptide is preferably comprised (as the first hybrid zinc finger domain) in a degron tag according to the invention, said degron tag according to the invention comprising two non-natural hybrid zinc finger domains (see FIG. 2).

Within the context of the current invention the degron tag according to the invention may also be referred to as ‘double hybrid degron’.

Within the context of the current invention a degron comprising a first hybrid zinc finger domain and a second non-hybrid zinc finger domain may also be referred to as ‘single hybrid, dual zinc finger degron’

Within the current invention a degron tag comprising a first hybrid zinc finger domain and no second zinc finger domain may also be referred to as ‘single hybrid, single zinc finger degron’.

In some embodiments the non-natural hybrid zinc finger polypeptide according to the invention is such ‘single hybrid, single zinc finger degron’ comprising two amino acid substitutions as described herein. In embodiments wherein the non-natural hybrid zinc finger polypeptide according to the invention, in addition to the first hybrid zinc finger domain, further comprises a second non-hybrid zinc finger domain, such molecule may also be referred to as single hybrid, dual zinc finger degron comprising at least two amino acid substitutions as described herein. In embodiments wherein the non-natural hybrid zinc finger polypeptide according to the invention, in addition to the first hybrid zinc finger domain, further comprises a second hybrid zinc finger domain, such molecule may also be referred to as ‘double hybrid degron’ comprising at least two amino acid substitutions as described herein.

In accordance with the invention as disclosed herein, and according to a first aspect there is provided for a degron tag comprising a (non-natural) first hybrid zinc finger domain and a (non-natural) second hybrid zinc finger domain wherein
(1) the first hybrid zinc finger domain comprises a first portion and a second portion;
(2) the first portion comprises the amino acid sequence X₁X₂C₃X₄X₅C₆X₇X₈X₉X₁₀X₁₁, wherein X represents any amino acid, of a first Cys₂-His₂ zinc finger domain;
(3) the second portion comprises the amino acid sequence X₁₂X₁₃X₁₄X₁₅X₁₆X₁₇X₁₈H₁₉X₂₀X₂₁X₂₂H₂₃, wherein X represents any amino acid, of a second Cys₂-His₂ zinc finger domain, wherein the second Cys₂-His₂ zinc finger domain is different from the first Cys₂-His₂ zinc finger domain;
(4) the second portion is C-terminal with respect to the first portion;
(5) the second hybrid zinc finger domain is C-terminal with respect to the first hybrid zinc finger domain;
(6) the second hybrid zinc finger domain comprises a third portion and a fourth portion;
(7) the third portion comprises the amino acid sequence (Z)₂C(Z)₂C(Z)₅₋₆ wherein Z represents any amino acid, of a third Cys₂-His₂ zinc finger domain;
(8) the fourth second portion comprises the amino acid sequence (Z)₆H(Z)₃₋₄H, wherein Z represents any amino acid, of a fourth Cys₂-His₂ zinc finger domain, wherein the fourth Cys₂-His₂ zinc finger domain is different from the third Cys₂-His₂ zinc finger domain; and
(9) the fourth portion is C-terminal with respect to the third portion; and
(10) wherein the first hybrid zinc finger domain comprises at least two amino acid substitutions, wherein an amino acid substitution in the first portion of the first hybrid zinc finger domain is relative to the first portion of the first Cys₂-His₂ zinc finger domain and wherein an amino acid substitution in the second portion of the first hybrid zinc finger domain is relative to the second portion of the second Cys₂-His₂ zinc finger domain, and wherein the substitution is not at any of positions C₃, C₆, X₇, H₁₉, or H₂₃.

The current invention provides for systems that may comprise zinc finger degrons herein also referred to as degron tags. Generally, a degron or degron tag is a peptide sequence or protein element that is involved in regulating the degradation rate of a protein that, for example, comprises such degron or degron tag. The degron tag according to the invention comprises, in some embodiments, two zinc finger domains. The term zinc finger domain is, within the context of the current invention, well understood by the skilled person. In embodiments, the degron is therefore a zinc finger degron comprising at least two zinc finger domains and that can be controlled with an IMID such as thalidomide, lenalidomide, pomalidomide, and/or analogs thereof. Preferably the degron tag is able to promote or control degradation of a POI, for example when fused to the degron tag, in the presence of such IMID. Preferably, one or both of the hybrid zinc finger domains in the degron tag according to the invention is/are non-natural hybrid zinc finger.

The first hybrid zinc finger domain that is comprised in a degron tag according to the invention comprises a first portion and a second portion. The first portion and the second portion each independently consist of an amino acid sequence. The first portion and the second portion that are comprised in the first hybrid zinc finger domain may or may not be directly adjacent to each other. In some embodiments the first portion and the second portion are connected via a linker peptide of, for example, one, two, three, four, five, or more amino acids. Therefore, is some embodiments the first portion and the second portion may be connected to each other via a further linker peptide, for example comprising one, two, three, four, five, six, seven or more amino acids. However, in some embodiments no additional linker peptide is present between the first portion of the first hybrid zinc finger domain and the second portion of the first hybrid zinc finger domain. In such embodiment the first and second portion are directly adjacent to each other. Preferably the first portion and the second portion are directly adjacent to each other.

The first portion of the first hybrid zinc finger domain may be N-terminal or may be C-terminal from the second portion of the first hybrid zinc finger domain. In a preferred embodiment the first portion of the first hybrid zinc finger domain is N-terminal of the second portion of the first hybrid zinc finger domain.

In a preferred embodiment the first portion consists of 8-30 amino acids, with increasing preference 11-30, 10-20, 11-20, 10-14, 11-14, 10-11, most preferably 11 amino acids.

In a preferred embodiment the second portion consists of 8-30 amino acids, with increasing preference 12-30, 11-20, 12-20, 11-14, 12-14, most preferably 12 amino acids.

In some embodiment, the, preferably non-natural, first hybrid zinc finger domain, consist of about 16-60 amino acids, preferably about 20-36 amino acids, about 20-30 amino acids, for example 20, 21, 22, 23, 24, 25, 26, 27, 28, 29 or 30 amino acids. In particular embodiments, the first hybrid zinc finger domain comprises a Cys₂ His₂ (C₂H₂) domain, and wherein in the hybrid zinc finger domain comprises at least two subdomains (herein referred to as portions), and wherein each subdomain is from a different wild type zinc finger. For example, wherein the first portion of the first hybrid zinc finger domain is from a first wild type zinc finger, and wherein the second portion of the first hybrid zinc finger domain is from a second, different, wild type zinc finger domain. Based on the disclosure herein the skilled person is well capable of selecting the first portion of the first hybrid zinc finger domain from a first wild type zinc finger and/or well capable of selecting the second portion of the first hybrid zinc finger domain from a second wild type zinc finger. For example, he may appropriately select such portions from wild type zinc finger domains (in particular wild type Cys₂-His₂ (C₂H₂) zinc finger domains) that are available from various scientific publications and public and well-known gene- and protein databases, for example from (Sievers et al. Science. 2018 Nov. 2; 362 (6414): eaat0572), and that are, for example known or expected to destabilize proteins (e.g., the wild type protein that comprise such wild-type zinc finger domain) in the presence of an IMiD such as thalidomide and therewith may control degradation of such protein. With respect to the second zinc finger, the second hybrid zinc finger and/or the third portion or fourth portion as described herein, in some embodiment, these on its own (i.e., in the absence of the first hybrid zinc finger domain) may or may not be sensitive to IMiDs, i.e., that may or may not destabilize proteins in the presence of an IMiD such as thalidomide and therewith may control degradation of such protein. Preferably it is not sensitive to IMiDs on its own.

In particular embodiments the first portion of the first hybrid zinc finger domain comprises an amino acid sequence represented by the sequence X₁X₂C₃X₄X₅C₆X₇X₈X₉X₁₀X₁₁, wherein each X (X₁, X₂, . . . , etc.) represents, independently, any amino acid, more in particular wherein each X represent any natural or proteinogenic amino acid (e.g. Alanine, Arginine, Asparagine, Aspartic Acid, Cysteine, Glutamic acid, Glutamine, Glycine, Histidine, Isoleucine, Leucine, Lysine, Methionine, Phenylalanine, Proline. Serine, Threonine, Tryptophan, Tyrosine, and Valine). An alternative way of representing the amino acid sequence comprised in the first portion of the first hybrid zinc finger domain is (X)₂C(X)₂C(X)₅. However, since amino acids substitutions at particular positions in, amongst others, the first portion of the first hybrid zinc finger domain, are part of the invention it is preferred to use X₁X₂C₃X₄X₅C₆X₇X₈X₉X₁₀X₁₁, indicating the individual amino acids and positions thereof.

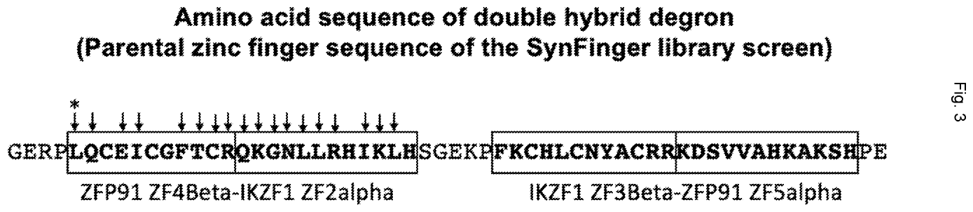

The skilled person will understand that, for example, X₁ may represent another amino acid than, for example X₂, or it may represent the same amino acid. The skilled person understands that C₃ indicates the presence of a cysteine at the third position in this amino acid sequence that is comprised in the first portion of the first hybrid zinc finger domain. Likewise, C₆ indicates the presence of a cysteine at the sixth position. In a preferred embodiment the first portion of the first hybrid zinc finger domain has the above indicated amino acid sequence (X₁X₂C₃X₄X₅C₆X₇X₈X₉X₁₀X₁₁) and wherein the amino acid sequence is of a first Cys₂-His₂ zinc finger domain (e.g., of a wild type Cys₂-His₂ zinc finger domain). In some embodiment, the indicated amino acid sequence of the first portion of the first hybrid zinc finger domain may comprise an additional short stretch of amino acids N-terminal thereof, for example 1-10, preferably 1-5, for example 1, 2, 3, 4, or 5 amino acids, for example of said first or second Cys₂-His₂ zinc finger domain. FIG. 3, for example, provides an example of a degron tag according to the invention (with positions of amino acid substitutions indicated) and wherein N-terminal of the first hybrid zinc finger domain there is a short stretch of amino acids GERP (in this case originating from the IKZF1; in some other embodiments the stretch may be from another wild type zinc finger domain, and may, for example be GEKP). Experiments confirmed that although such short stretch may be included, it does not substantially affect the results obtained with the degron tags (and/or hybrid zinc finger polypeptides according to the invention).

As detailed herein elsewhere, the first hybrid zinc finger domain comprised in the degron tag according to the invention is in preferred embodiments a hybrid zinc finger comprising a Cys2 His2 (C2H2) zinc finger domain and wherein the hybrid zinc finger domain comprises at least two subdomains, i.e., a first portion and a second portion, and wherein the amino acid sequence of the first portion is from a wild type zinc finger, more in particular a wild type Cys2 His2 (C2H2) zinc finger.

In particular embodiments the second portion of the first hybrid zinc finger domain comprises an amino acid sequence represented by the sequence X₁₂X₁₃X₁₄X₁₅X₁₆X₁₇X₁₈H₁₉X₂₀X₂₁X₂₂H₂₃, wherein each X (X₁₂, X₁₃, . . . , etc.) represents, independently, any amino acid, more in particular wherein each X represent any natural or proteinogenic amino acid (e.g. Alanine, Arginine, Asparagine, Aspartic Acid, Cysteine, Glutamic acid, Glutamine, Glycine, Histidine, Isoleucine, Leucine, Lysine, Methionine, Phenylalanine, Proline. Serine, Threonine, Tryptophan, Tyrosine, and Valine). An alternative way of representing the amino acid sequence comprised in the second portion of the first hybrid zinc finger domain is (X)₇H(X)₃H. However, since amino acids substitutions at particular positions in, amongst others, the second portion of the first hybrid zinc finger domain, are part of the invention it is preferred to use X₁₂X₁₃X₁₄X₁₅X₁₆X₁₇X₁₈H₁₉X₂₀X₂₁X₂₂H₂₃., indicating the individual amino acids and positions thereof. In some embodiments it is also contemplated that the second portion of the first hybrid zinc finger domain is (X)₆H(X)₃H.

The skilled person will understand that, for example, X₁₂ may represent another amino acid than, for example X₁₃, or it may represent the same amino acid. The skilled person understands that H₁₉ indicates the presence of a histidine at the nineteenth position in this amino acid sequence that is comprised in the second portion of the first hybrid zinc finger domain. Likewise, H₂₃ indicates the presence of a histidine cysteine at the twenty-third position in this sequence. In a preferred embodiment the second portion of the first hybrid zinc finger domain has the above indicated amino acid sequence (X₁₂X₁₃X₁₄X₁₅X₁₆X₁₇X₁₈H₁₉X₂₀X₂₁X₂₂H₂₃) and wherein the amino acid sequence is of a second Cys₂-His₂ zinc finger domain.

In some embodiment, the indicated amino acid sequence of the second portion of the first hybrid zinc finger domain may comprise an additional short stretch of amino acids C-terminal thereof, for example 1-10, preferably 1-5, for example 1, 2, 3, 4, or 5 amino acids, for example of said second Cys₂-His₂ zinc finger domain. In preferred embodiments the first portion of the first hybrid zinc finger domain is N-terminal from the second portion of the first hybrid zinc finger domain. In such embodiment, the second portion is C-terminal with respect to the first portion.

In other words, the first hybrid zinc finger domain comprised in the degron tag according to the invention is in preferred embodiments a hybrid zinc finger domain comprising a Cys2 His2 (C2H2) domain and wherein the hybrid zinc finger domain comprises at least two subdomains, i.e. a first portion and a second portion, and wherein the amino acid sequence of the first portion is from a wild type zinc finger, more in particular a wild type Cys2 His2 (C2H2) zinc finger (i.e. the first Cys₂-His₂ zinc finger domain), and wherein the amino acid sequence of the second portion is from a wild type zinc finger, more in particular a wild type Cys2 His2 (C2H2) zinc finger (i.e. the second Cys2-His2 zinc finger domain), and wherein, preferably, the second Cys₂-His₂ zinc finger domain is different from the first Cys₂-His₂ zinc finger domain (for example, wherein the first Cys₂-His₂ zinc finger domain is a different wild type zinc finger than the second Cys₂-His₂ zinc finger domain). Preferably the amino acid sequence of the first portion comprises or is X₁X₂C₃X₄X₅C₆X₇X₈X₉X₁₀X₁ and/or the amino acid sequence of the second portion comprises or is X₁₂X₁₃X₁₄X₁₅X₁₆X₁₇X₁₈H₁₉X₂₀X₂₁X₂₂H₂₃.

As indicated above, the first portion and the second portion together form the first hybrid zinc finger domain, and wherein the first portion is preferably of a first Cys₂-His₂ zinc finger domain, e.g., of a wild type, naturally occurring Cys₂-His₂ zinc finger domain and the second portion is preferably of a second, different, Cys₂-His₂ zinc finger domain, e.g., of a different wild type, naturally occurring Cys₂-His₂ zinc finger domain. In such embodiments, the first hybrid zinc finger domain that is formed by the first portion and the second portion may be referred to as a hybrid Cys₂-His₂ (C2H2) zinc finger domain, e.g., a Cys₂-His₂ (C2H2) zinc finger domain that does not occur in wild type zinc finger proteins and/or in nature.

The C2H2 zinc finger domain structure has been found to be an important determinant in binding and may even be a more important determining factor than the primary amino acid sequence of the zinc finger domain (see, for example Sievers et al. Science. 2018 Nov. 2; 362 (6414): eaat0572). The C2H2 zinc finger domains are known as recurrent motifs that mediate drug-dependent (e.g., IMiDs) interactions with ubiquitin ligase CRL4^{CRBN} (see, for example, An et al, Nat Commun. 8:15398 (2017), doi: 10.1038/ncommsI5398, Koduri et al, PNAS 116 (7) 2539-2544 (2019), doi: 10.1073/pnas.1818109116, WO2019089592, WO2020132039, WO2021188286, or WO202108042).

Typically, a C2H2 zinc finger domain, including e.g., the first hybrid zinc finger domain and/or the second (hybrid) zinc finger domain according to the invention, comprise beta-hairpin and alpha-helix subdomains. Typically, a C2H2 zinc finger domain consist of about 20-36 amino acids, about 20-30 amino acids, for example 20, 21, 22, 23, 24, 25, 26, 27, 28, 29 or 30 amino acids. A C2H2 zinc finger domain typically comprises an N-terminal beta-hairpin comprising two conserved cysteine residues, followed by an alpha helix comprising two conserved histidine residues at its C-terminus (see, for example Fedotova et al., ActaNaturae, 2017 April-June; 9 (2): 47-58). Said motif is also present in the zinc finger domains according to the invention, including the first and the second hybrid zinc finger domain.

In addition to the first hybrid (non-natural) zinc finger domain, the degron tag according to the invention may also comprises a second (non-natural) hybrid zinc finger domain. In the degron tag according to the invention said second hybrid zinc finger domain is C-terminal with respect to the first hybrid zinc finger domain. In such embodiment the first hybrid zinc finger domain in the degron tag according to the invention is N-terminal with respect to the second hybrid zinc finger domain.

In some embodiment the first hybrid zinc finger domain and the second hybrid zinc finger domain are directly adjacent to each other, with no peptide linker being present. In some embodiments the first hybrid zinc finger domain and the second hybrid zinc finger domains are not directly adjacent to each other but connected to each other via a short peptide linker. In embodiments wherein the first hybrid zinc finger domain and the second hybrid zinc finger domain are connected via a linker peptide, such linker peptide may be comprised of, for example, one, two, three, four, five, six, seven, or more amino acids.

Therefore, is some embodiments the first hybrid zinc finger domain may be connected to the second hybrid zinc finger domain via a further linker peptide, for example comprising one, two, three, four, five, six, seven or more amino acids. However, in some embodiments no additional linker peptide is present.

For example, in some embodiments the linker may be a short stretch of amino acids that originate from a wild-type zinc finger domain, for example from the N-terminal part or adjacent to the N-terminal part of a wild type zinc finger domain. Such amino acid sequence may, for example, be a SGEKP sequence (see FIG. 3) (in this case originating from the IKZF1 protein—SGEKP sequence is the endogenous linker between IKZF1 ZF2 and IKZF1 ZF3 in the IKZF1 protein; in the same manner other endogenous linker may, for example, be used as linker within the context of the current invention, for example comparable endogenous linkers from other C2H2 zinc finger proteins); in some other embodiments the stretch may be from another wild type zinc finger domain, and may, for example be GERP). Experiments confirmed that although such short stretch may be included, it does not substantially affect the results obtained with the degron tags (and/or hybrid zinc finger polypeptides according to the invention), and the skilled person is well able to provide additional linker suitable within the context of the current invention.

With respect to the second hybrid zinc finger domain, it has been found that including such second hybrid zinc finger domain may further improve the sensitivity of the degron tag of the invention in controlling protein degradation, protein levels and or cellular activity. According to this aspect of the invention there is therefore provided for a degron tag wherein the degron tag comprises a first and a second hybrid zinc finger domain as detailed herein.

The second hybrid zinc finger domain comprises a third portion and a fourth portion. The third portion and the fourth portion each independently consist of an amino acid sequence. The third portion and the fourth portion that are comprised in the second hybrid zinc finger domain may or may not be directly adjacent to each other. In some embodiments the third portion and the fourth portion are connected via a linker peptide of, for example, one, two, three, four, five, or more amino acids. Therefore, is some embodiments the third portion and the fourth portion may be connected to each other via a further linker peptide, for example comprising one, two, three, four, five, six, seven or more amino acids. However, in some embodiments no additional linker peptide is present between the third portion of the second hybrid zinc finger domain and the fourth portion of the second hybrid zinc finger domain. In such embodiment the third and fourth portion are directly adjacent to each other. Preferably the third portion and the fourth portion are directly adjacent to each other.

The third portion of the second hybrid zinc finger domain may be N-terminal or may be C-terminal from the fourth portion of the second hybrid zinc finger domain. In a preferred embodiment the third portion is N-terminal of the fourth portion.

In a preferred embodiment the third portion consists of 8-30 amino acids, with increasing preference 11-30, 10-20, 11-20, 10-14, 11-14, 10-11, most preferably 11 amino acids.

In a preferred embodiment the fourth portion consists of 8-30 amino acids, with increasing preference 12-30, 11-20, 12-20, 11-14, 12-14, most preferably 12 amino acids.

In some embodiment, the, preferably non-natural, second hybrid zinc finger domain, consist of about 16-60 amino acids, preferably about 20-36 amino acids, about 20-30 amino acids, for example 20, 21, 22, 23, 24, 25, 26, 27, 28, 29 or 30 amino acids. In particular embodiments, the second hybrid zinc finger domain comprises a Cys2 His2 (C2H2) domain, and wherein in the hybrid zinc finger domain comprises at least two subdomains (herein referred to as portions), and wherein each subdomain is from a different wild type zinc finger. For example, wherein the third portion of the second hybrid zinc finger domain is from a third wild type zinc finger, and wherein the fourth portion of the second hybrid zinc finger domain is from a fourth, different, wild type zinc finger domain. Based on the disclosure herein the skilled person is well capable of selecting the third portion of the second hybrid zinc finger domain from a third wild type zinc finger and/or well capable of selecting the fourth portion of the second hybrid zinc finger domain from a fourth wild type zinc finger. For example, he may appropriately select such portions from wild type zinc finger domains (in particular wild type Cys2-His2 (C2H2) zinc finger domains) that are available from various scientific publications and public and well-known gene- and protein databases, for example from (Sievers et al. Science. 2018 Nov. 2; 362 (6414): eaat0572), and that are, for example known or expected to destabilize proteins (e.g., the wild type protein that comprise such wild-type zinc finger domain) in the presence of an IMiD such as thalidomide and therewith may control degradation of such protein.

In particular embodiments the third portion of the second hybrid zinc finger domain comprises an amino acid sequence represented by the sequence (Z)₂C(Z)₂C(Z)₅₋₆, wherein Z represents any amino acid. (Z)₂ thus represent two amino acids whereas (Z)₅₋₆ indicates 5 or 6 amino acids. By way of example, the third portion of the second hybrid zinc finger domain may, for example, comprise an amino acid sequence Z₁Z₂C₃Z₄Z₅C₆Z₇Z₈Z₉Z₁₀Z₁₁ or Z₁Z₂C₃Z₄Z₅C₆Z₇Z₈Z₉Z₁₀Z₁₁Z₁₂ and wherein each Z (Z₁, Z₂, . . . , etc.) represents, independently, any amino acid, more in particular wherein each Z represent any natural or proteinogenic amino acid (e.g. Alanine, Arginine, Asparagine, Aspartic Acid, Cysteine, Glutamic acid, Glutamine, Glycine, Histidine, Isoleucine, Leucine, Lysine, Methionine, Phenylalanine, Proline. Serine, Threonine, Tryptophan, Tyrosine, and Valine). The skilled person will understand that, for example, Z₁ may represent another amino acid than, for example Z₂, or it may represent the same amino acid. The skilled person understands that C₃ indicates the presence of a cysteine at the third position in this amino acid sequence that is comprised in the third portion of the second hybrid zinc finger domain. Likewise, C₆ indicates the presence of a cysteine at the sixth position. In a preferred embodiment the third portion of the second hybrid zinc finger domain has the above indicated amino acid sequence (Z)₂C(Z)₂C(Z)₅₋₆, even more preferably (Z)₂C(Z)₂C(Z)₆ and wherein the amino acid sequence is of a third Cys₂-His₂ zinc finger domain (e.g., of a wild type Cys2-His2 zinc finger domain). In some embodiment, the indicated amino acid sequence of the third portion of the second hybrid zinc finger domain may comprise an additional short stretch of amino acids N-terminal thereof, for example 1-10, preferably 1-5, for example 1, 2, 3, 4, or 5 amino acids, for example of said third or fourth (wildtype) Cys2-His2 zinc finger domain. FIG. 3, for example, provides an example of a degron tag according to the invention (with positions of amino acid substitutions indicated) and wherein N-terminal of the second hybrid zinc finger domain there is a short stretch of amino acids SGEKP, and as discussed above. Experiments confirmed that although such short stretch may be included, it does not substantially affect the results obtained with the degron tags (and/or hybrid zinc finger polypeptides according to the invention).

As detailed herein, and comparable to the first hybrid zinc finger domain comprised in the degron tag according to the invention, also the second hybrid zinc finger domain is in preferred embodiments a hybrid zinc finger comprising a Cys2 His2 (C2H2) zinc finger domain and wherein the hybrid zinc finger domain comprises at least two subdomains, i.e. a third portion and a fourth portion, and wherein the amino acid sequence of the third portion is from a wild type zinc finger, more in particular a wild type Cys2 His2 (C2H2) zinc finger.

In particular embodiments the fourth portion of the second hybrid zinc finger domain comprises an amino acid sequence represented by the sequence (Z)₆H(Z)₃₋₄H. (Z)₆ thus represent six amino acids whereas (Z)₃₋₄ indicates 3 or 4 amino acids By way of example, the fourth portion of the second hybrid zinc finger domain may, for example, comprise an amino acid sequence Z₁₃Z₁₄Z₁₅Z₁₆Z₁₇Z₁₈H₁₉Z₂₀Z₂₁Z₂₂H₂₃ or Z₁₃Z₁₄Z₁₅Z₁₆Z₁₇Z₁₈H₁₉Z₂₀Z₂₁Z₂₂Z₂₃H₂₄, wherein each Z (Z₁₃, Z₁₄, . . . , etc.) represents, independently, any amino acid, more in particular wherein each Z represent any natural or proteinogenic amino acid (e.g. Alanine, Arginine, Asparagine, Aspartic Acid, Cysteine, Glutamic acid, Glutamine, Glycine, Histidine, Isoleucine, Leucine, Lysine, Methionine, Phenylalanine, Proline. Serine, Threonine, Tryptophan, Tyrosine, and Valine). The skilled person will understand that, for example, Z₁₃ may represent another amino acid than, for example Z₁₄, or it may represent the same amino acid. The skilled person understands that H₁₉ indicates the presence of a histidine at the nineteenth position in this amino acid sequence that is comprised in the second portion of the first hybrid zinc finger domain. Likewise, H₂₃ or H₂₄ indicates the presence of a histidine cysteine at the twenty-third position in this sequence or the twenty-fourth position in this sequence (depending on whether (Z)₃₋₄ relates to 3 or 4 amino acids). It is noted that, for example, Z₁₃ does not indicate a position relative to the first hybrid zinc finger domain, since it is, for example, explained herein elsewhere that in some embodiments there may be linkers between the first hybrid zinc finger domain and the second (hybrid) zinc finger domain. It is also explained herein that the length of the third portion of the second (hybrid) zinc finger may vary, and may, for example be 11 amino acids in lengths. In a preferred embodiment the fourth portion of the second hybrid zinc finger domain has the above indicated amino acid sequence (Z)₆H(Z)₃₋₄H and wherein the amino acid sequence is of a second Cys2-His2 zinc finger domain, i.e., more in particular a wild type Cys2 His2 (C2H2) zinc finger.

In other words, the second hybrid zinc finger domain comprised in the degron tag according to the invention is in preferred embodiments a hybrid zinc finger domain comprising a Cys2 His2 (C2H2) domain and wherein the hybrid zinc finger domain comprises at least two subdomains, i.e. a third portion and a fourth portion, and wherein the amino acid sequence of the third portion is from a wild type zinc finger, more in particular a wild type Cys2 His2 (C2H2) zinc finger (i.e. the third Cys2-His2 zinc finger domain), and wherein the amino acid sequence of the fourth portion is from a wild type zinc finger, more in particular a wild type Cys2 His2 (C2H2) zinc finger (i.e. the fourth Cys2-His2 zinc finger domain), and wherein, preferably, the third Cys2-His2 zinc finger domain is different from the fourth Cys2-His2 zinc finger domain (for example, wherein the third Cys2-His2 zinc finger domain is a different wild type zinc finger than the fourth Cys2-His2 zinc finger domain). Preferably the amino acid sequence of the third portion comprises or is (Z)₂C(Z)₂C(Z)₅₋₆ and/or the amino acid sequence of the fourth portion comprises or is (Z)₆H(Z)₃₋₄H.

In preferred embodiment the fourth portion is C-terminal with respect to the third portion.

As will be understood by the skilled person, in addition to the domain, portions and linkers described above, the degron tag according to the invention may comprise further amino acid, for example N-terminal from the first hybrid zinc finger domain or C-terminal form the second hybrid zinc finger domain.

In preferred embodiments, the degron tag according to the invention is further characterized by the presence of at least two amino acid substitutions in the first hybrid zinc finger domain, and wherein an amino acid substitution in the first portion of the first hybrid zinc finger domain is relative to the first portion of the first Cys₂-His₂ zinc finger domain and wherein an amino acid substitution in the second portion of the first hybrid zinc finger domain is relative to the second portion of the second Cys₂-His₂ zinc finger domain, and wherein the substitution is not at any of positions C₃, C₆, X₇, H₁₉, or H₂₃. In some embodiments X₇ is a glycine.

As discussed herein, in embodiments, the first hybrid zinc finger domain is comprised from a first portion that is from a first Cys₂-His₂ zinc finger domain, in particular from a first wild type Cys₂-His₂ zinc finger domain, and wherein the first portion comprises the amino acid sequence X₁X₂C₃X₄X₅C₆X₇X₈X₉X₁₀X₁₁ (i.e., as present in the first wild type Cys₂-His₂ zinc finger domain). As discussed herein, in embodiments, the first hybrid zinc finger domain is comprised from a second portion that is from a second Cys₂-His₂ zinc finger domain, in particular from a second wild type Cys₂-His₂ zinc finger domain, and wherein the second portion comprises the amino acid sequence X₁₂X₁₃X₁₄X₁₅X₁₆X₁₇X₁₈H₁₉X₂₀X₂₁X₂₂H₂₃ (i.e., as present in the second wild type Cys₂-His₂ zinc finger domain).

In those embodiments wherein the degron tag according to the invention is further characterized by the presence of at least two amino acid substitutions in the first hybrid zinc finger domain, and wherein an amino acid substitution is in the first portion of the first hybrid zinc finger domain, this amino acid substitution is an amino acid substitution relative to the first portion of the first Cys₂-His₂ zinc finger domain from which the sequence was obtained. For example, if X₂ is a Valine in the first (wildtype) Cys₂-His₂ zinc finger domain from which the sequence was obtained, and this Valine is substituted with, for example, a Lysine in the degron tag according to the invention, in the degron tag according to the invention X₂ will be a Lysine and the amino acid substitution to a Lysine is relative to the Valine that was comprised in the wild type Cys₂-His₂ zinc finger domain from which it was obtained. In a similar way an amino acid substitution in the second portion of the first hybrid zinc finger domain is defined herein. For example, if X₂₀ is a Proline in the second (wildtype) Cys₂-His₂ zinc finger domain from which the sequence for the second portion was obtained, and this Proline is substituted with, for example, a Leucine in the degron tag according to the invention, in the degron tag according to the invention X₂₀ will be a Leucine and the amino acid substitution to a Leucine is relative to the Proline that was comprised in the wild type Cys₂-His₂ zinc finger domain from which it was obtained.

As disclosed above, relative to the portions from the wild type first Cys₂-His₂ zinc finger domain and second Cys₂-His₂ zinc finger domain, the first hybrid zinc finger domain comprises at least two amino acid substitutions. The skilled person is well capable of providing, preparing and recognizing such amino acid substitutions relative to the wild type first Cys₂-His₂ zinc finger domain and second Cys₂-His₂ zinc finger domain, for example by comparing the amino acid sequence of the first portion and or the second portion of the first hybrid zinc finger domain therewith.

In some embodiment the at least two amino acids substitutions (i.e., replacement of an amino acid from the wild type sequence with another, different, amino acid) are both in the first portion. In some embodiments the at least two amino acid substitutions are both in the second portion. In some other embodiment one amino acid substitution is in the first portion and one amino acid substitutions is in the second portion. In some preferred embodiments, the first hybrid zinc finger domain comprises no more than two amino acid substitutions. In some preferred embodiments, the first hybrid zinc finger domain comprises more than two amino acid substitutions, for example, 3, 4, 5, or 6 amino acid substitutions, preferably 2, 3 or 4 amino acid substitutions. In some preferred embodiments, the first zinc finger domain comprises (exactly) two amino acid substitutions, i.e. no more than two amino acid substitutions. In some preferred embodiments, the first zinc finger domain comprises (exactly) three amino acid substitutions, i.e. no more than three amino acid substitutions. In some preferred embodiments, the first zinc finger domain comprises (exactly) four amino acid substitutions, i.e. no more than four amino acid substitutions. In those embodiments wherein the first hybrid zinc finger domain comprises more than two amino acid substitutions, for example three or four amino acid substitutions, in preferred embodiments, the for example third and/or fourth amino acid substitution is selected or also selected from the substitutions as disclosed herein, for example as listed in Table 1. In those embodiments wherein the first hybrid zinc finger domain comprise, for example, four amino acid substitutions (or six), in preferred embodiments, the four amino acid substitutions (or six) are selected from the pair of substitutions disclosed herein, for example as listed in Table 2 (for example Q12R K13V G14N K21A) and/or the pair of position and amino acids to be substituted as listed in Table 4. In a preferred embodiment, as is discussed herein, and wherein the first hybrid zinc finger domain comprises 4 amino acid substitutions, the first hybrid zinc finger domain according to the invention comprises an amino acid sequence according to SEQ ID NO: 135 (LQCEICGFTCRRVNNLLRHIALH; i.e. quadruple substitutions Q12R/K13V/G14N/K21A) and/or the degron tag according to the invention comprises an amino acid sequence according to SEQ ID NO: 136 (LQCEICGFTCRRVNNLLRHIALHSGEKPFKCHLCNYACRRKDSVVAHKAKSH).

In a preferred embodiment, the substitution is not in any of the positions C₃, C₆, X₇, H₁₉, or H₂₃. In some embodiments X₇ is G₇.

It was surprisingly found that by introducing the at least two amino acid substitutions in the first and/or second portions of the first hybrid zinc finger domain, and relative to the first portion of the (wild-type) first Cys₂-His₂ zinc finger domain and the second portion of the (wild-type) second Cys₂-His₂ zinc finger domain, degron tags can be provided and that have highly enhanced or increased sensitivity to an IMiD molecule, e.g. thalidomide or analogue, relative to a wild-type zinc finger domain and relative to a hybrid zinc finger domain but that is comprised of a wild-type subdomains (as described herein) and do not comprise the at least two amino acid substitutions. Interestingly, although it was found that introducing one amino acid substitutions may also enhanced or increased sensitivity to an ImiD molecule, unexpectedly introducing a further amino acid substitution may even further enhance or increase sensitivity to an ImiD molecule, as is shown in the examples and described herein.

According to this aspect of the invention there is therefor provided for degron tags comprising at least a first hybrid zinc finger domain comprising at least two amino acid substitutions relative to the wild type zinc finger domains (portions thereof) from which the hybrid zinc finger domain is composed, and that has enhanced or increased sensitivity to an ImiD molecule.

In some embodiments the at least two amino acid substitutions are at position that are directly adjacent to each other, in other embodiments, the positions of the amino acid substitutions are separated from each other by at least 1, 2, 3, 4, 5, 6, 7, 8, 10, 11, 12, 13 or more amino acids (i.e., non-substituted amino acids), for example as shown in the accompanying tables, figures and examples.

Although in principle any amino acid substitutions is allowed, preferably the amino acid substitutions on a particular position in the first hybrid zinc finger domain is one as described herein (with respect to either the amino acid substituted or with respect to the substituting amino acid). The skilled person is, based on the information disclosed herein, and without due burden, able to provide for such amino acid substitutions, and degron tags according to the invention.

Without tending to be bound by theory, the current inventors' belief that the enhanced or increased sensitivity to an ImiD molecule that is observed with the degron tags and/or hybrid zinc finger polypeptides according to the invention is at least in part due to altered or improved interaction of the degron tag, cereblon and the immunomodulatory drug (IMID). Therefore, in preferred embodiments, there is provided for a degron tag according to the invention and/or a hybrid zinc finger polypeptide according to the invention and wherein the degron tag and/or the hybrid zinc finger polypeptide is able to bind a complex formed between cereblon (CRBN) and an immunomodulatory drug (IMiD).

Also provided is for a degron tag and/or hybrid zinc finger polypeptide according to the invention, wherein the first portion is a beta-hairpin loop of a first Cys₂-His₂ zinc finger domain, the second portion is an alpha-helix region of a second Cys₂-His₂ zinc finger domain, the third portion is a beta-hairpin loop of a third Cys₂-His₂ zinc finger domain, and/or the fourth portion is an alpha-helix region of a fourth Cys₂-His₂ zinc finger domain. As explained above the term “first, second, third or fourth Cys₂-His₂ zinc finger domain” refer to Cys₂-His₂ zinc finger domains as these are found in nature, i.e., Cys₂-His₂ zinc finger domains from wild type zinc finger proteins. As explained above, in the degron tag and/or hybrid zinc finger polypeptide according to the invention, the first hybrid zinc finger domain may comprise at least two amino acid substitutions. As explained above, portions of these wild type Cys₂-His₂ zinc finger domains are used in providing the degron tag and/or hybrid zinc finger polypeptide according to the invention. According to a preferred embodiment of the invention the first portion that is used is a beta-hairpin loop of a first Cys₂-His₂ zinc finger domain, the second portion that is used is an alpha-helix region of a second Cys₂-His₂ zinc finger domain, the third portion that is used is a beta-hairpin loop of a third Cys₂-His₂ zinc finger domain, and/or the fourth portion that is used is an alpha-helix region of a fourth Cys₂-His₂ zinc finger domain. Zinc finger domains include a beta-hairpin loop and an alpha-helix region, and the skilled person knows how to provide for a beta-hairpin loop portion and/or an alpha-helix region portion of (wildtype) Cys₂-His₂ zinc finger domains. Representative examples are well-known to the skilled person and include, for example, those disclosed and described herein. Examples of zinc finger comprising a beta-hairpin loop and an alpha-helix region include but are not limited (human) IKZF1, IKZF2, IKZF3, SALL4, ZFP91, GZF1, ZNF653, ZNF692, ZNF827, ZBTB39, WIZ, ZNF98, ZNF654, ZNF787, ZNF 276, ZNF582, ZNF517 and E4F1 (see also Uniprot Accession no: Q13422, Q9UKS7, Q9UKT9, Q9UJQ4, Q96JP5, Q9H116, Q96CK0, Q9BU19, Q17R98, 015060, O95785, A6NK75, Q8IZM8, Q6DD87, Q8N554, Q96NG8, Q6ZMY9, Q66K89). As disclosed above, the degron tag and/or hybrid zinc finger polypeptide according to the invention may include one or more amino acid residues N-terminal with respect to the beta-hairpin portion, one or more amino acid residues between the beta-hairpin portion and the alpha-helix portion, and one or more amino acid residues C-terminal with respect to the alpha-helix portion provided that the degron tag and/or hybrid zinc finger polypeptide according to the invention is a substrate for a CRBN-IMiD complex, and/or shows enhanced or increased sensitivity to an IMiD molecule (relative to e.g. to wild type situation). These additional amino acids may correspond to residues in the native Cys₂-His₂ zinc finger domains or be different provided that the degron tag maintains a zinc finger-like fold and exhibits the properties as disclosed herein.

In embodiments of the invention, there is provided for the degron tag and/or hybrid zinc finger polypeptide according to the invention wherein the first substitution is in the second portion and the second substitution is in the first portion or in the second portion. Although the at least two amino acid substitutions may both be comprised in the first portion, or may both be comprised in the second portion, or one may be comprised in the first portion and the second may be comprised in the second portion, in a preferred embodiment, at least one of the amino acid substitutions is present in the second portion. It was found that in particular suitable degron tags and/or hybrid zinc finger polypeptides according to the invention can be provided and wherein at least one of the amino acid substitutions is in the second portion, i.e., is in the alpha helix portion of the first hybrid zinc finger domain. The second amino acid substitutions may be present in either the second portion or may be present in the first portion (i.e., in the beta hairpin loop).

There is also provided for the degron tag and/or hybrid zinc finger polypeptide according to the invention and wherein at least one substitution is at a position selected from the group consisting of X₁, X₄, X₁₂, X₁₃, X₁₄, X₁₅, X₁₇, X₂₁, and X₂₂. It was found that in particular an amino acid substitution on one of these position in the first hybrid zinc finger domain allows for providing degron tag and/or hybrid zinc finger polypeptide according to the invention with increased or enhanced sensitivity to an IMiD molecule. Without being bound by theory it is believed that in particular these positions are important positions in the first hybrid zinc finger domain that allow for the observed improved or increased sensitivity to an IMID, as shown in the Examples. Preferably the substituting amino acid is as disclosed herein.

In preferred embodiments, there is provided for a degron tag and/or hybrid zinc finger polypeptide according to the invention, and wherein at least one of the substitutions that is present in the degron tag and/or hybrid zinc finger polypeptide according to the invention (or that is present in the first hybrid zinc finger domain) is selected from those listed in Table 1. Table 1 list the various position X₁-X₂₂ and where a substitution in the degron tag and/or hybrid zinc finger polypeptide according to the invention may, in preferred embodiments, be present, together with the substituting amino acid (i.e., the amino acid that is included at that position in the degron tag and/or hybrid zinc finger polypeptide according to the invention; one letter code). Preferably both amino acid substitutions (both position and substituting amino acids) are selected from those listed in Table 1. The skilled person will understand that when both amino acid substitutions are selected from those listed in Table 1 (or any other Table of list provided herein), each amino acid substitution is at a different position. At the same time the skilled person will understand that a substituting amino acid is meant to indicate an amino acid that is different from the native amino acid (i.e., that is different from that as present in the wild type Cys₂-His₂ zinc finger domain used for the first or second portion of the first hybrid zinc finger domain). In that respect, it is noted that the current invention is not in particular limited to a particular wild type Cys₂-His₂ zinc finger domain used for providing the first portion, second portion (and/or third and fourth portion as discussed herein).

Likewise, the current invention also encompasses the use of the positions and substitutions as listed in Table 1 (or any other Table of list provided herein)-in any suitable first or second portion of a (wildtype) Cys₂-His₂ zinc finger domain that may be used in providing for the first hybrid zinc finger domain according to the invention. In other words, the positions and substituting amino acids listed in Table 1 (or any other Table of list provided herein) are applicable to any suitable first hybrid zinc finger domain as long the degron tag and/or hybrid zinc finger polypeptide according to the invention maintains a zinc finger-like fold and exhibits the properties as disclosed herein (e.g., sensitivity to IMiD in the context of protein degradation).

However, in a preferred embodiment the position and substitutions are relative to the first hybrid zinc finger domain as used in the examples herein, i.e., wherein the first portion of the first hybrid zinc finger domain is a beta-hairpin region of ZFP91ZF4, and the second portion of the first hybrid zinc finger domain is an alpha-helix region of IKZF1 ZF2 (see FIG. 3; wherein the first hybrid zinc finger domain of the ‘parental degron tag’ used in the examples is schematically represented).

TABLE 1
Preferred positions and substitutions (substituting amino acid)

```
  1F
  1H
  1I
  1M
  1W
  1Y
  4A
  4D
  4H
  4I
  4K
  4L
  4M
  4N
  4P
  4Q
  4R
  4V
  4W
  5A
  5V
  8Y
  9K
  9Q
  9R
  9S
10A
10F
10S
11K
11V
12H
12K
12L
12N
12R
12V
13D
13E
13H
13L
13M
13P
13Q
13R
13T
13V
13W
13Y
14A
14F
14H
14K
14L
14M
14N
14Q
14R
14S
14T
14V
14Y
15A
15K
15R
15S
15T
15V
15W
16M
16Y
17D
17H
17I
17K
17M
17R
17V
17W
17Y
18K
18L
18V
18W
20K
20R
20Y
21A
21F
21H
21I
21L
21N
21R
21S
21Y
22H
22K
22N
22Q
22R
22T
```

In preferred embodiments there is provided for a degron tag and/or hybrid zinc finger polypeptide according to the invention, and wherein the at least two substitutions are selected from those listed in Table 2. Table 2 lists preferred combinations of two amino acid substitutions at two different position according to the invention in the first hybrid zinc finger domain as described herein (showing per row a preferred combination of two positions within the first hybrid zinc finger domain and the corresponding substituting amino acids; for example 4R and 12L indicates that in this combination the amino acid at position 4 in the first hybrid zinc finger domain is substituted with an Arginine (R) and the amino acid at position 12 in the first hybrid zinc finger domain is substituted with a Leucine (L)-in other words the original amino acids in the portions obtained from the wild type Cys2-His2 zinc finger domains are substituted with an Arginine in position 4 and an Leucine at position 12). In preferred embodiment of the invention, the at least two substitutions are selected from any of the combinations shown in Table 2.

TABLE 2
preferred combination of positions and
substitutions (substituting amino acid)

| Substitution1 | Substitution 2 | Substitution1 | Substitution 2 |
|---|---|---|---|
| 4R | 12L | 14K | 21S |
| 12R | 13T | 1F | 14R |
| 12R | 17M | 20Y | 22T |
| 14M | 15R | 18V | 21A |
| 13V | 14K | 14F | 15K |
| 14N | 21A | 4K | 12H |
| 14N | 22R | 14M | 17V |
| 13L | 18W | 4R | 16M |
| 12R | 13V | 12L | 18L |
| 4W | 21Y | 13W | 16Y |
| 4P | 14S | 1W | 13Y |
| 1I | 5A | 13P | 22R |
| 16M | 21R | 4W | 12R |
| 17I | 21L | 17H | 18L |
| 4L | 12R | 21F | 22K |
| 12R | 17Y | 4V | 17V |
| 4I | 18K | 13Q | 14L |
| 4A | 10S | 1H | 15R |
| 12L | 21L | 17D | 22R |
| 4K | 12V | 4W | 14M |
| 9S | 17R | 12K | 14N |
| 4K | 10S | 1Y | 21I |
| 12K | 15A | 4H | 10S |
| 14H | 22N | 1W | 21A |
| 4K | 13M | 4H | 13H |
| 1F | 14V | 4Q | 12R |
| 11V | 21A | 12K | 13R |
| 12R | 22H | 4N | 13L |
| 13V | 15W | 12K | 21I |
| 4M | 10F | 13P | 20Y |
| 14T | 15R | 13D | 15R |
| 13E | 14K | 4M | 17M |
| 4D | 18L | 4W | 16M |
| 14Y | 21S | 15K | 16Y |
| 10A | 12R | 1W | 4M |
| 1Y | 4V | 4V | 9Q |
| 1F | 14H | 14Q | 15R |
| 8Y | 12K | 9R | 22K |
| 15R | 22Q | 1M | 14S |
| 11K | 14N | 13H | 15V |
| 5V | 12K | 4W | 17W |
| 12R | 20R | 13L | 14R |
| 1F | 15R | 10S | 15R |
| 14A | 17K | 1M | 4V |
| 4I | 17W | 4L | 21R |
| 17Y | 20Y | 14L | 22R |
| 15T | 22R | 12K | 14V |
| 12K | 20K | 9K | 22R |
| 12R | 15S | 21N | 22N |
| 12N | 14K | 15R | 21H |

The skilled person will understand that a substituting amino acid is meant to indicate an amino acid that is different from the native amino acid (i.e., that is different from that as present in the wild type Cys₂-His₂ zinc finger domain used for the first or second portion of the first hybrid zinc finger domain). In that respect, it is noted that the current invention is not in particular limited to a particular wild type Cys₂-His₂ zinc finger domain used for providing the first portion, second portion (and/or third and fourth portion as discussed herein).

Likewise, the current invention also encompasses the use of the positions and substitutions as listed in Table 2 (or any other Table of list provided herein)-in any suitable first or second portion of a (wildtype) Cys₂-His₂ zinc finger domain that may be used in providing for the first hybrid zinc finger domain according to the invention. In other words, the positions and substituting amino acids listed in Table 2 (or any other Table of list provided herein) are applicable to any suitable first hybrid zinc finger domain as long the degron tag and/or hybrid zinc finger polypeptide according to the invention maintains a zinc finger-like fold and exhibits the properties as disclosed herein (e.g., sensitivity to IMiD in the context of protein degradation).

However, in a preferred embodiment the position and substitutions are relative to the first hybrid zinc finger domain as used in the examples herein, i.e., wherein the first portion of the first hybrid zinc finger domain is a beta-hairpin region of ZFP91ZF4, and the second portion of the first hybrid zinc finger domain is an alpha-helix region of IKZF1 ZF2 (see FIG. 3; wherein the first hybrid zinc finger domain of the ‘parental degron tag’ used in the examples is schematically represented).

Furthermore, the frequently observed primary amino acid substitutions Q12R, Q12K, N15R and L22R in the examples, increased sensitivity towards IMiDs further in a synergistic manner when combined with the secondary amino acid substitutions listed in Table 8 (see below), (i.e., EI Q12R/K13V=0.006877267, whereas EI Q12R=0.167217557). Thus in particular preferred embodiments of the invention, a combination of amino acid substitutions are mentioned in table 8 is used/provided, e.g. 12R and 13T or 22R and 14L, and so on.

In preferred embodiments there is provided for a degron tag and/or hybrid zinc finger polypeptide according to the invention, and wherein an amino acid in the first portion that is substituted and/or an amino acid in the second portion that is substituted is selected from those listed in Table 3.

Table 3 list preferred amino acids that are substituted in the first hybrid zinc finger domain of the degron tag and/or hybrid zinc finger polypeptide according to the invention. In other words, in a preferred embodiment of the invention, the position and/or the position and the amino acid that is substituted is selected from those listed in Table 3. Accordingly, according to a preferred embodiment of the current invention, the at least two amino acid substitutions are selected from a combination of the position 1, 4, 5, 8, 9, 10, 11, 12, 13, 14, 15, 16, 17, 18, 20, 21 and 22, and wherein the amino acids that are substituted are a combination of two amino acids as listed in Table 3.

TABLE 3
preferred position and amino acid to be substituted

L1
E4
I5
F8
T9
C10
R11
Q12
K13
G14
N15
L16
L17
R18
I20
K21
L22

Preferably both amino acids that are substituted (both position and substituting amino acids) are selected from those listed in Table 3. As will be understood by the skilled person the amino acids, for example the preferred amino acids as shows in Table 3 may be substituted with any suitable amino acid as long as it provides for a degron tag and/or hybrid zinc finger polypeptide according to the invention. However, in a preferred embodiment, the amino acids, preferably a combination of amino acids listed in Table 3 are replaced with a corresponding amino acid as listed in Table 1 or corresponding combination of amino acids as shown in Table 2 (wherein corresponding refers, for example, to the corresponding position in the first hybrid zinc finger domain as disclosed herein).

The skilled person will understand that when both amino acids that are substituted are selected from those listed in Table 3 (or any other Table of list provided herein), each amino acid substitution is at a different position. At the same time the skilled person will understand that a substituted amino acid is meant to indicate an amino acid that is substituted with another amino acids and that is different from the native amino acid (i.e., that is different from that is present in the wild type Cys2-His2 zinc finger domain used for the first or second portion of the first hybrid zinc finger domain). In that respect, it is noted that the current invention is not in particular limited to a particular wild type Cys2-His2 zinc finger domain used for providing the first portion, second portion (and/or third and fourth portion as discussed herein).

Likewise, the current invention also encompasses the use of the positions and substitutions as listed in Table 3 (or any other Table or list provided herein) in any suitable first or second portion of a (wildtype) Cys2-His2 zinc finger domain that may be used in providing for the first hybrid zinc finger domain according to the invention. In other words, the positions and amino acids that are substituted that are listed in Table 3 (or any other Table of list provided herein) are applicable to any suitable first hybrid zinc finger domain as long the degron tag and/or hybrid zinc finger polypeptide according to the invention maintains a zinc finger-like fold and exhibits the properties as disclosed herein (e.g., sensitivity to IMiD in the context of protein degradation).

However, in a preferred embodiment the position and substitutions are relative to the first hybrid zinc finger domain as used in the examples herein, i.e., wherein the first portion of the first hybrid zinc finger domain is a beta-hairpin region of ZFP91 ZF4 and the second portion of the first hybrid zinc finger domain is an alpha-helix region of IKZF1 ZF2 (see FIG. 3; wherein the first hybrid zinc finger domain of the ‘parental degron tag’ used in the examples is schematically represented).

In preferred embodiments there is provided for a degron tag and/or hybrid zinc finger polypeptide according to the invention, and wherein the at least two amino acids in the first portion and/or in the second portion that are substituted are selected from those listed in Table 4.

Table 4 lists preferred combinations of two amino acids that are substituted at two different position according to the invention in the first hybrid zinc finger domain as described herein (showing per row a preferred combination of two positions within the first hybrid zinc finger domain and the corresponding substituting amino acids; for example E4 and Q12 indicates that in this combination the amino acid at position 4 in the first hybrid zinc finger domain that is substituted is Glutamic acid (E) and that the amino acid at position 12 that is substituted is Glutamine (Q)—in other words the original amino acids in the portions obtained from the wild type Cys2-His2 zinc finger domains are Glutamic acid as position 4 and Glutamine at position 12. In a preferred embodiment of the invention, the at least two amino acids that are substituted are selected from any of the combinations shown in Table 4. As will be understood by the skilled person the amino acids, for example the preferred amino acids as shows in Table 4 may be substituted with any suitable amino acid as long as it provides for a degron tag and/or hybrid zinc finger polypeptide according to the invention. However, in a preferred embodiment, the amino acids, preferably the combination of amino acids listed in Table 4 are replaced with a corresponding amino acid as listed in Table 1 or corresponding combination of amino acids as shown in Table 2 (wherein corresponding refers, for example, to the corresponding position in the first hybrid zinc finger domain as disclosed herein).

TABLE 4
preferred combinations of position
and amino acids to be substituted.

| Amino acid 1 | Amino acid 2 | Amino acid 1 | Amino acid 2 |
|---|---|---|---|
| E4 | Q12 | G14 | K21 |
| Q12 | K13 | L1 | G14 |
| Q12 | L17 | I20 | L22 |
| G14 | N15 | R18 | K21 |
| K13 | G14 | G14 | N15 |
| G14 | K21 | E4 | Q12 |
| G14 | L22 | G14 | L17 |
| K13 | R18 | E4 | L16 |
| Q12 | K13 | Q12 | R18 |
| E4 | K21 | K13 | L16 |
| E4 | G14 | L1 | K13 |
| L1 | I5 | K13 | L22 |
| L16 | K21 | E4 | Q12 |
| L17 | K21 | L17 | R18 |
| E4 | Q12 | K21 | L22 |
| Q12 | L17 | E4 | L17 |
| E4 | R18 | K13 | G14 |
| E4 | C10 | L1 | N15 |
| Q12 | K21 | L17 | L22 |
| E4 | Q12 | E4 | G14 |
| T9 | L17 | Q12 | G14 |
| E4 | C10 | L1 | K21 |
| Q12 | N15 | E4 | C10 |
| G14 | L22 | L1 | K21 |
| E4 | K13 | E4 | K13 |
| L1 | G14 | E4 | Q12 |
| R11 | K21 | Q12 | K13 |
| Q12 | L22 | E4 | K13 |
| K13 | N15 | Q12 | K21 |
| E4 | C10 | K13 | I20 |
| G14 | N15 | K13 | N15 |
| K13 | G14 | E4 | L17 |
| E4 | R18 | E4 | L16 |
| G14 | K21 | N15 | L16 |
| C10 | Q12 | L1 | E4 |
| L1 | E4 | E4 | T9 |
| L1 | G14 | G14 | N15 |
| F8 | Q12 | T9 | L22 |
| N15 | L22 | L1 | G14 |
| R11 | G14 | K13 | N15 |
| I5 | Q12 | E4 | L17 |
| Q12 | I20 | K13 | G14 |
| L1 | N15 | C10 | N15 |
| G14 | L17 | L1 | E4 |
| E4 | L17 | E4 | K21 |
| L17 | I20 | G14 | L22 |
| N15 | L22 | Q12 | G14 |
| Q12 | I20 | T9 | L22 |
| Q12 | N15 | K21 | L22 |
| Q12 | G14 | N15 | K21 |

The skilled person will understand that a substituting amino acid is meant to indicate an amino acid that is different from the native amino acid that is substituted (i.e., that is different from that as present in the wild type Cys2-His2 zinc finger domain used for the first or second portion of the first hybrid zinc finger domain). In that respect, it is noted that the current invention is not in particular limited to a particular wild type Cys2-His2 zinc finger domain used for providing the first portion, second portion (and/or third and fourth portion as discussed herein).

Likewise, the current invention also encompasses the use of the positions and substitutions as listed in Table 4 (or any other Table of list provided herein) in any suitable first or second portion of a (wildtype) Cys2-His2 zinc finger domain that may be used in providing for the first hybrid zinc finger domain according to the invention. In other words, the positions and amino acids that are substituted and listed in Table 4 (or any other Table of list provided herein) are applicable to any suitable first hybrid zinc finger domain as long the degron tag and/or hybrid zinc finger polypeptide according to the invention maintains a zinc finger-like fold and exhibits the properties as disclosed herein (e.g., sensitivity to IMiD in the context of protein degradation).

However, in a preferred embodiment the position and amino acids that are substituted are relative to the first hybrid zinc finger domain as used in the examples herein, i.e., wherein the first portion of the first hybrid zinc finger domain is a beta-hairpin region of ZFP91 ZF4 and the second portion of the first hybrid zinc finger domain is an alpha-helix region of IKZF1 ZF2 (see FIG. 3; wherein the first hybrid zinc finger domain of the ‘parental degron tag’ used in the examples is schematically represented).

In further embodiments there is provided for a degron tag and/or hybrid zinc finger polypeptide according to the invention, and wherein
(1) the first Cys₂-His₂ zinc finger domain is selected from the group consisting of IKZF1 ZF2, IKZF3 ZF2, ZFP91 ZF4, ZNF654 ZF1, ZNF787 ZF5, ZNF653 ZF4, ZNF276 ZF4, ZNF692 ZF4, ZNF582 ZF9, ZNF517 ZF10, E4F1 ZF2, and ZNF827 ZF1, preferably ZFP91 ZF4;
(2) the second Cys₂-His₂ zinc finger domain is selected from the group consisting of IKZF1 ZF2, IKZF3 ZF2, ZFP91 ZF4, ZNF654 ZF1, ZNF787 ZF5, ZNF653 ZF4, ZNF276 ZF4, ZNF692 ZF4, ZNF582 ZF9, ZNF517 ZF10, E4F1 ZF2, ZNF827 ZF1, preferably IKZF1 ZF2;
(3) the third Cys₂-His₂ zinc finger domain is selected from the group consisting of IKZF1 ZF3, IKZF3 ZF3, ZFP91 ZF5, ZNF653 ZF5, ZNF276 ZF5, ZNF827 ZF2, ZNF692 ZF5, preferably IKZF1 ZF3; and/or
(4) the fourth Cys₂-His₂ zinc finger domain is selected from the group consisting of IKZF1 ZF3, IKZF3 ZF3, ZFP91 ZF5, ZNF653 ZF5, ZNF276 ZF5, ZNF827 ZF2, ZNF692 ZF5, preferably ZFP91 ZF5.

The current invention is not in particular limited to specific (wild type) Cys₂-His₂ zinc finger domains that provide for the first, second, third and/or fourth portion in, respectively, the first hybrid zinc finger domain and the second zinc finger domain, in particular second hybrid zinc finger domain. In the art various wild-types or naturally occurring Cys₂-His₂ zinc finger domains have been described and that are suitable in degron systems that include the use of IMiDs (see for example those described by (Sievers et al. Science. 2018 Nov. 2; 362 (6414): eaat0572)). The skilled person will appreciate that such Cys₂-His₂ zinc finger domains, comprising the typical beta hairpin loop and the alpha helix, can therefore suitably be used in the current invention and provide, for example, the first, second, third and/or fourth portion in, respectively, the first hybrid zinc finger domain and the second zinc finger domain, in particular second hybrid zinc finger domain, and wherein in the first and/or second portion at least two amino acids may be substituted and detailed herein. Therefore, such degron tags and/or hybrid zinc finger polypeptides according to the invention that maintain a zinc finger-like fold (beta-hairpin loop and alpha helix) and exhibits the properties as disclosed herein (e.g., sensitivity to IMiD in the context of protein degradation) can be easily provided for by the skilled person.

However, in a preferred embodiment
(1) the first Cys₂-His₂ zinc finger domain is selected from the group consisting of IKZF1 ZF2, IKZF3 ZF2, ZFP91 ZF4, ZNF654 ZF1, ZNF787 ZF5, ZNF653 ZF4, ZNF276 ZF4, ZNF692 ZF4, ZNF582 ZF9, ZNF517 ZF10, E4F1 ZF2, and ZNF827 ZF1, preferably ZFP91 ZF4;
(2) the second Cys₂-His₂ zinc finger domain is selected from the group consisting of IKZF1 ZF2, IKZF3 ZF2, ZFP91 ZF4, ZNF654 ZF1, ZNF787 ZF5, ZNF653 ZF4, ZNF276 ZF4, ZNF692 ZF4, ZNF582 ZF9, ZNF517 ZF10, E4F1 ZF2, ZNF827 ZF1, preferably IKZF1 ZF2;
(3) the third Cys₂-His₂ zinc finger domain is selected from the group consisting of IKZF1 ZF3, IKZF3 ZF3, ZFP91 ZF5, ZNF653 ZF5, ZNF276 ZF5, ZNF827 ZF2, ZNF692 ZF5, preferably IKZF1 ZF3; and/or
(4) the fourth Cys₂-His₂ zinc finger domain is selected from the group consisting of IKZF1 ZF3, IKZF3 ZF3, ZFP91 ZF5, ZNF653 ZF5, ZNF276 ZF5, ZNF827 ZF2, ZNF692 ZF5, preferably ZFP91 ZF5.

In other words, the amino acid sequence of the Cys₂-His₂ zinc finger domains for selection of a first portion and/or second portion (see below) is in some embodiments selected from the group consisting of: SEQ ID NO: 116 (IKZF1 ZF2-FQCNQCGASFTQKGNLLRHIKLH), SEQ ID NO: 117 (IKZF3 ZF2-FQCNQCGASFTQKGNLLRHIKLH), SEQ ID NO: 118 (ZFP91 ZF4-LQCEICGFTCRQKASLNWHMKKH), SEQ ID NO: 119 (ZNF654 ZF1-FACVICGRKFRNRGLMQKHLKNH), SEQ ID NO: 120 (ZNF787 ZF5-FVCPRCGRGFSQPKSLARHLRLH), SEQ ID NO: 121 (ZNF653 ZF4-LQCEICGYQCRQRASLNWHMKKH), SEQ ID NO: 122 (ZNF276 ZF4-LQCEVCGFQCRQRASLKYHMTKH), SEQ ID NO: 123 (ZNF692 ZF4-LQCEICGFTCRQKASLNWHQRKH), SEQ ID NO: 124 (ZNF582 ZF9-YQCKVCGRAFKRVSHLTVHYRIH), SEQ ID NO: 125 (ZNF517 ZF 10-YRCRACGRACSRLSTLIQHQKVH), SEQ ID NO: 126 (E4F1 ZF2-HECKLCGASFRTKGSLIRHHRRH), and/or SEQ ID NO: 127 (ZNF827 ZF1-FQCPICGLVIKRKSYWKRHMVIH).

In other words, the amino acid sequence of the Cys2-His2 zinc finger domains for selection of a third portion and/or fourth portion (see below) is in some embodiments selected from the group consisting of: SEQ ID NO: 128 (IKZF1 ZF3-FKCHLCNYACRRRDALTGHLRTH), SEQ ID NO: 129 (IKZF3 ZF3-FKCHLCNYACQRRDALTGHLRTH), SEQ ID NO: 130 (ZFP91 ZF5-FSCNICGKKFEKKDSVVAHKAKSH), SEQ ID NO: 131 (ZNF653 ZF5-FTCDRCGKRFEKLDSVKFHTLKSH), SEQ ID NO: 132 (ZNF276 ZF5-FACDQCGRRFEKAHNLNVHMSMVH), SEQ ID NO: 133 (ZNF827 ZF2-HQCPLCPFRCARKDNLKSHMKVH), and/or SEQ ID NO: 134 (ZNF692 ZF5-FPCEFCGKRFEKPDSVAAHRSKSH).

According to this embodiment, the first Cys₂-His₂ zinc finger domain provides for the first portion of the first hybrid zinc finger domain and is selected from those listed above.

According to this embodiment, the second Cys₂-His₂ zinc finger domain provides for the second portion of the first hybrid zinc finger domain and is selected from those listed above.

In the degron tag and/or hybrid zinc finger polypeptide according to the invention these first and second portion provides for the first hybrid zinc finger domain and further comprises at least two amino acid substitutions relative to the first and/or second portion provided by the first and second Cys₂-His₂ zinc finger domain.

According to this embodiment, the third Cys₂-His₂ zinc finger domain provides for the third portion of the second hybrid zinc finger domain and is selected from those listed above.

According to this embodiment, the fourth Cys₂-His₂ zinc finger domain provides for the fourth portion of the second hybrid zinc finger domain and is selected from those listed above. As is discussed herein the second zinc finger in the degron tag and/or hybrid zinc finger polypeptide according to the invention may in certain embodiment be a hybrid zinc finger domain or may be a non-hybrid zinc finger domain. In the latter case, the second zinc finger may therefor also be selected from any of the third or fourth Cys₂-His₂ zinc finger domain zinc finger domains listed above (providing both the first portion and the second portion from the same Cys₂-His₂ zinc finger domain).

In a preferred embodiment at least one, two, three or four of the first, second, third and fourth Cys₂-His₂ zinc finger domain listed above are selected. In a preferred embodiment all of the first, second, third and fourth Cys₂-His₂ zinc finger domain listed above are selected. In a preferred embodiment at least the first and the second Cys₂-His₂ zinc finger domain is selected from those listed above. In a preferred embodiment at least the third and fourth Cys₂-His₂ zinc finger domain is selected from those listed above.

In a highly preferred embodiment, the beta hairpin loop (or first portion) of the first hybrid zinc finger domain is from ZFP91 ZF4 and the alpha helix (or second portion) of the first hybrid zinc finger is from IKZF1 ZF2. In a further highly preferred embodiment, the beta hairpin loop (or third portion) of the second hybrid zinc finger domain is from IKZF1 ZF3 and the alpha helix (or fourth portion) of the second hybrid zinc finger is from ZFP91 ZF5.

In some embodiments one of the first and second Cys₂-His₂ zinc finger domain and one of the third and fourth Cys₂-His₂ zinc finger domain are from the same zinc finger protein (e.g., a ZF4 domain from ZFP91 and a ZF5 domain from ZFP91). In some embodiment, the first and second Cys₂-His₂ zinc finger domain are from two different zinc finger protein, and the third and fourth Cys₂-His₂ zinc finger domain are from the same two different zinc finger proteins (for example the first Cys₂-His₂ zinc finger domain and the fourth Cys₂-His₂ zinc finger domain are both from ZFP91 (respectively zinc finger domain ZF4 and ZF5) and the second Cys₂-His₂ zinc finger domain and the third Cys₂-His₂ zinc finger domain are both from a different zinc finger protein, for example IKZF1 (respectively zinc finger domain ZF2 and ZF3). When two different Cys₂-His₂ zinc finger domains from the same zinc finger protein are provided, the Cys₂-His₂ zinc finger domain that in the wild type zinc finger protein is N-terminal orientated relative to the other is preferably also N-terminal from the other in the degron tags and/or hybrid zinc finger polypeptides according to the invention.

The listed Cys₂-His₂ zinc finger domains, there sequence and structure are well-known to the skilled person and available from various scientific publications and databases.

As already briefly discussed, in an embodiment of the invention there is also provided for the degron tags and/or hybrid zinc finger polypeptides according to the invention wherein the at least two amino acid substitutions are introduced in a first hybrid zinc finger domain the first portion thereof having an amino acid sequence according to SEQ ID NO: 101 (LQCEICGFTCR-ZFP91 ZF4 (first portion)), and/or the second portion thereof having an amino acid sequence according to SEQ ID NO: 102 (QKGNLLRHIKLH-IKZF1 ZF2 (second portion)), and/or the first hybrid zinc finger domain having an amino acid sequence according to SEQ ID NO: 103 (LQCEICGFTCRQKGNLLRHIKLH-(ZFP91 ZF4/IKZF1 ZF2 first hybrid zinc finger domain)). In preferred embodiments, in these sequences the at least two amino acid substitutions according to the invention may be introduced.

In a further preferred embodiment, there is provided for the degron tags and/or hybrid zinc finger polypeptides according to the invention wherein the at least two amino acid substitutions are introduced (e.g. having any of the sequences according to SEQ ID NO: 101-103), and wherein the third portion thereof having an amino acid sequence according to SEQ ID NO: 104 (FKCHLCNYACRR-(IKZF1 ZF3 (third portion)), and/or the fourth portion thereof having an amino acid sequence according to SEQ ID NO: 105 (KDSVVAHKAKSH (ZFP91 ZF5 (fourth portion)), and/or the second hybrid zinc finger domain having an amino acid sequence according to SEQ ID NO: 106 (FKCHLCNYACRRKDSVVAHKAKSH-Second hybrid zinc finger domain).

In a further preferred embodiment, there is provided for the degron tags and/or hybrid zinc finger polypeptides according to the invention wherein the at least two amino acid substitutions are introduced, and wherein the degron tags and/or hybrid zinc finger polypeptides has an amino acid sequence according to SEQ ID NO: 107 (LQCEICGFTCRQKGNLLRHIKLHSGEKPFKCHLCNYACRRKDSVVAHKAKSH). In preferred embodiments, in these sequences the at least two amino acid substitutions according to the invention may be introduced.

In preferred embodiments there is provided for a degron tag and/or hybrid zinc finger polypeptide according to the invention, and wherein the first hybrid zinc finger domain comprising the two substitutions is selected from those listed in Table 5. Table 5 list preferred first hybrid zinc finger domains (or hybrid zinc finger polypeptides) according to the invention. Also SEQ ID NO: 135 is a preferred first hybrid zinc finger domain (or hybrid zinc finger polypeptides) according to the invention. SEQ ID NO: 136 is comprised in a preferred degron tag according to the invention. These preferred zinc finger domains are those that showed the most beneficial EI index (enrichment index) as calculated according to the example section. The results thereof are summarized in Table 6.

TABLE 5
Preferred first hybrid zinc finger domains

| | SEQ ID |
|---|---|
| Sequence | NO. |
| LQCRICGFTCRLKGNLLRHIKLH | 1 |
| LQCEICGFTCRRTGNLLRHIKLH | 2 |
| LQCEICGFTCRRKGNLMRHIKLH | 3 |
| LQCEICGFTCRQKMRLLRHIKLH | 4 |
| LQCEICGFTCRQVKNLLRHIKLH | 5 |
| LQCEICGFTCRQKNNLLRHIALH | 6 |
| LQCEICGFTCRQKNNLLRHIKRH | 7 |
| LQCEICGFTCRQLGNLLWHIKLH | 8 |
| LQCEICGFTCRRVGNLLRHIKLH | 9 |
| LQCWICGFTCRQKGNLLRHIYLH | 10 |
| LQCPICGFTCRQKSNLLRHIKLH | 11 |
| IQCEACGFTCRQKGNLLRHIKLH | 12 |
| LQCEICGFTCRQKGNMLRHIRLH | 13 |
| LQCEICGFTCRQKGNLIRHILLH | 14 |
| LQCLICGFTCRRKGNLLRHIKLH | 15 |
| LQCEICGFTCRRKGNLYRHIKLH | 16 |
| LQCIICGFTCRQKGNLLKHIKLH | 17 |
| LQCAICGFTSRQKGNLLRHIKLH | 18 |
| LQCEICGFTCRLKGNLLRHILLH | 19 |
| LQCKICGFTCRVKGNLLRHIKLH | 20 |
| LQCEICGFSCRQKGNLRRHIKLH | 21 |
| LQCKICGFTSRQKGNLLRHIKLH | 22 |
| LQCEICGFTCRKKGALLRHIKLH | 23 |
| LQCEICGFTCRQKHNLLRHIKNH | 24 |
| LQCKICGFTCRQMGNLLRHIKLH | 25 |
| FQCEICGFTCRQKVNLLRHIKLH | 26 |
| LQCEICGFTCVQKGNLLRHIALH | 27 |
| LQCEICGFTCRRKGNLLRHIKHH | 28 |
| LQCEICGFTCRQVGWLLRHIKLH | 29 |
| LQCMICGFTFRQKGNLLRHIKLH | 30 |
| LQCEICGFTCRQKTRLLRHIKLH | 31 |
| LQCEICGFTCRQEKNLLRHIKLH | 32 |
| LQCDICGFTCRQKGNLLLHIKLH | 33 |
| LQCEICGFTCRQKYNLLRHISLH | 34 |
| LQCEICGFTARRKGNLLRHIKLH | 35 |
| YQCVICGFTCRQKGNLLRHIKLH | 36 |
| FQCEICGFTCRQKHNLLRHIKLH | 37 |
| LQCEICGYTCRKKGNLLRHIKLH | 38 |
| LQCEICGFTCRQKGRLLRHIKQH | 39 |
| LQCEICGFTCKQKKNLLRHIKLH | 40 |
| LQCEVCGFTCRKKGNLLRHIKLH | 41 |
| LQCEICGFTCRRKGNLLRHRKLH | 42 |
| FQCEICGFTCRQKGRLLRHIKLH | 43 |
| LQCEICGFTCRQKANLKRHIKLH | 44 |
| LQCIICGFTCRQKGNLWRHIKLH | 45 |
| LQCEICGFTCRQKGNLYRHYKLH | 46 |
| LQCEICGFTCRQKGTLLRHIKRH | 47 |
| LQCEICGFTCRKKGNLLRHKKLH | 48 |
| LQCEICGFTCRRKGSLLRHIKLH | 49 |
| LQCEICGFTCRNKKNLLRHIKLH | 50 |
| LQCEICGFTCRQKKNLLRHISLH | 51 |
| FQCEICGFTCRQKRNLLRHIKLH | 52 |
| LQCEICGFTCRQKGNLLRHYKTH | 53 |
| LQCEICGFTCRQKGNLLVHIALH | 54 |
| LQCEICGFTCRQKFKLLRHIKLH | 55 |
| LQCKICGFTCRHKGNLLRHIKLH | 56 |
| LQCEICGFTCRQKMNLVRHIKLH | 57 |
| LQCRICGFTCRQKGNMLRHIKLH | 58 |
| LQCEICGFTCRLKGNLLLHIKLH | 59 |
| LQCEICGFTCRQWGNYLRHIKLH | 60 |
| WQCEICGFTCRQYGNLLRHIKLH | 61 |
| LQCEICGFTCRQPGNLLRHIKRH | 62 |
| LQCWICGFTCRRKGNLLRHIKLH | 63 |
| LQCEICGFTCRQKGNLHLHIKLH | 64 |
| LQCEICGFTCRQKGNLLRHIFKH | 65 |
| LQCVICGFTCRQKGNLVRHIKLH | 66 |
| LQCEICGFTCRQQLNLLRHIKLH | 67 |
| HQCEICGFTCRQKGRLLRHIKLH | 68 |
| LQCEICGFTCRQKGNLDRHIKRH | 69 |
| LQCWICGFTCRQKMNLLRHIKLH | 70 |
| LQCEICGFTCRKKNNLLRHIKLH | 71 |
| YQCEICGFTCRQKGNLLRHIILH | 72 |
| LQCHICGFTSRQKGNLLRHIKLH | 73 |
| WQCEICGFTCRQKGNLLRHIALH | 74 |
| LQCHICGFTCRQHGNLLRHIKLH | 75 |
| LQCQICGFTCRRKGNLLRHIKLH | 76 |
| LQCEICGFTCRKRGNLLRHIKLH | 77 |
| LQCNICGFTCRQLGNLLRHIKLH | 78 |
| LQCEICGFTCRKKGNLLRHIILH | 79 |
| LQCEICGFTCRQPGNLLRHYKLH | 80 |
| LQCEICGFTCRQDGRLLRHIKLH | 81 |
| LQCMICGFTCRQKGNLMRHIKLH | 82 |
| LQCWICGFTCRQKGNMLRHIKLH | 83 |
| LQCEICGFTCRQKGKYLRHIKLH | 84 |
| WQCMICGFTCRQKGNLLRHIKLH | 85 |
| LQCVICGFQCRQKGNLLRHIKLH | 86 |
| LQCEICGFTCRQKQRLLRHIKLH | 87 |
| LQCEICGFRCRQKGNLLRHIKKH | 88 |
| MQCEICGFTCRQKSNLLRHIKLH | 89 |
| LQCEICGFTCRQHGVLLRHIKLH | 90 |
| LQCWICGFTCRQKGNLWRHIKLH | 91 |
| LQCEICGFTCRQLRNLLRHIKLH | 92 |
| LQCEICGFTSRQKGRLLRHIKLH | 93 |
| MQCVICGFTCRQKGNLLRHIKLH | 94 |
| LQCLICGFTCRQKGNLLRHIRLH | 95 |
| LQCEICGFTCRQKLNLLRHIKRH | 96 |
| LQCEICGFTCRKKVNLLRHIKLH | 97 |
| LQCEICGFKCRQKGNLLRHIKRH | 98 |
| LQCEICGFTCRQKGNLLRHINNH | 99 |
| LQCEICGFTCRQKGRLLRHIHLH | 100 |

TABLE 6
EI index (enrichment index) of the preferred first
hybrid zinc finger domains listed in Table 5.

| SEQ ID | | | |
|---|---|---|---|
| No. | Sub. 1 | Sub. 2 | EI index |
| 1 | E4R | Q12L | 0 |
| 2 | Q12R | K13T | 0.00291222 |
| 3 | Q12R | L17M | 0.00372528 |
| 4 | G14M | N15R | 0.004109971 |
| 5 | K13V | G14K | 0.004648074 |
| 6 | G14N | K21A | 0.00471865 |
| 7 | G14N | L22R | 0.004968515 |
| 8 | K13L | R18W | 0.006186869 |
| 9 | Q12R | K13V | 0.006877267 |
| 10 | E4W | K21Y | 0.006950013 |
| 11 | E4P | G14S | 0.008068234 |
| 12 | L1I | I5A | 0.0085639 |
| 13 | L16M | K21R | 0.00876145 |
| 14 | L17I | K21L | 0.009984402 |
| 15 | E4L | Q12R | 0.010142772 |
| 16 | Q12R | L17Y | 0.010704273 |
| 17 | E4I | R18K | 0.010796113 |
| 18 | E4A | C10S | 0.011326673 |
| 19 | Q12L | K21L | 0.011767926 |
| 20 | E4K | Q12V | 0.012162914 |
| 21 | T9S | L17R | 0.012551551 |
| 22 | E4K | C10S | 0.013111269 |
| 23 | Q12K | N15A | 0.013300837 |
| 24 | G14H | L22N | 0.013512849 |
| 25 | E4K | K13M | 0.013833226 |
| 26 | L1F | G14V | 0.014775966 |
| 27 | R11V | K21A | 0.014865307 |
| 28 | Q12R | L22H | 0.015890989 |
| 29 | K13V | N15W | 0.016086794 |
| 30 | E4M | C10F | 0.016725195 |
| 31 | G14T | N15R | 0.016863083 |
| 32 | K13E | G14K | 0.016927083 |
| 33 | E4D | R18L | 0.017054368 |
| 34 | G14Y | K21S | 0.017225632 |
| 35 | C10A | Q12R | 0.017544896 |
| 36 | L1Y | E4V | 0.017694595 |
| 37 | L1F | G14H | 0.017868996 |
| 38 | F8Y | Q12K | 0.017942936 |
| 39 | N15R | L22Q | 0.01900977 |
| 40 | R11K | G14N | 0.01915403 |
| 41 | I5V | Q12K | 0.019438445 |
| 42 | Q12R | I20R | 0.019646848 |
| 43 | L1F | N15R | 0.019766277 |
| 44 | G14A | L17K | 0.020643322 |
| 45 | E4I | L17W | 0.020901639 |
| 46 | L17Y | I20Y | 0.020996596 |
| 47 | N15T | L22R | 0.022086922 |
| 48 | Q12K | I20K | 0.022190694 |
| 49 | Q12R | N15S | 0.022681348 |
| 50 | Q12N | G14K | 0.023070252 |
| 51 | G14K | K21S | 0.023086608 |
| 52 | L1F | G14R | 0.023099481 |
| 53 | I20Y | L22T | 0.023631198 |
| 54 | R18V | K21A | 0.024078298 |
| 55 | G14F | N15K | 0.024697019 |
| 56 | E4K | Q12H | 0.025430522 |
| 57 | G14M | L17V | 0.02583464 |
| 58 | E4R | L16M | 0.025841131 |
| 59 | Q12L | R18L | 0.026815642 |
| 60 | K13W | L16Y | 0.027389937 |
| 61 | L1W | K13Y | 0.027600513 |
| 62 | K13P | L22R | 0.027669711 |
| 63 | E4W | Q12R | 0.028290744 |
| 64 | L17H | R18L | 0.029469901 |
| 65 | K21F | L22K | 0.029885865 |
| 66 | E4V | L17V | 0.029894605 |
| 67 | K13Q | G14L | 0.030300224 |
| 68 | L1H | N15R | 0.031406463 |
| 69 | L17D | L22R | 0.031878391 |
| 70 | E4W | G14M | 0.031924717 |
| 71 | Q12K | G14N | 0.032269961 |
| 72 | L1Y | K21I | 0.032746025 |
| 73 | E4H | C10S | 0.033034775 |
| 74 | L1W | K21A | 0.033618582 |
| 75 | E4H | K13H | 0.03442029 |
| 76 | E4Q | Q12R | 0.034449761 |
| 77 | Q12K | K13R | 0.035310273 |
| 78 | E4N | K13L | 0.035438461 |
| 79 | Q12K | K21I | 0.035691832 |
| 80 | K13P | I20Y | 0.036848485 |
| 81 | K13D | N15R | 0.037261051 |
| 82 | E4M | L17M | 0.037538608 |
| 83 | E4W | L16M | 0.038387133 |
| 84 | N15K | L16Y | 0.039025335 |
| 85 | L1W | E4M | 0.039238539 |
| 86 | E4V | T9Q | 0.039295393 |
| 87 | G14Q | N15R | 0.039396688 |
| 88 | T9R | L22K | 0.039473684 |
| 89 | L1M | G14S | 0.040650091 |
| 90 | K13H | N15V | 0.0410691 |
| 91 | E4W | L17W | 0.04124576 |
| 92 | K13L | G14R | 0.041287832 |
| 93 | C10S | N15R | 0.04175191 |
| 94 | L1M | E4V | 0.042291085 |
| 95 | E4L | K21R | 0.042847145 |
| 96 | G14L | L22R | 0.042849089 |
| 97 | Q12K | G14V | 0.042938713 |
| 98 | T9K | L22R | 0.043024631 |
| 99 | K21N | L22N | 0.043386844 |
| 100 | N15R | K21H | 0.044608879 |

Indeed, it was surprisingly found that with the at least two substitutions in the first hybrid zinc finger domain as described herein, degron tags and/or hybrid zinc finger polypeptides according to the invention are provided that have enhanced or increased sensitivity of IMiDs, in particular in comparison to, for example, those that comprise only one substitution in the first hybrid zinc finger domain. For example, when the first hybrid zinc finger domain as used in the example comprised only one mutation (a substitution L1Y, providing sequence YQCEICGFTCRQKGNLLRHIKLH (SEQ ID NO: 108), the correspond EI value was at least 10-15 times higher (=indicate for a degron tag and/or hybrid zinc finger polypeptide that is less sensitive to an IMiD) as compared to the EI value determined for SEQ ID. NO 36 (and at least 5 times as compared to SEQ ID. NO 100). This thus shows that despite introducing a second substitution, or more, for example a third or fourth amino acid substitution, in the first hybrid zinc finger domain, sensitivity of the resulting first hybrid zinc finger domain (having at least two substitutions) is surprisingly and dramatically improved. Exemplary EI values obtained with various single mutations in the first hybrid single domain used in the examples herein are show in Table 7.

TABLE 7
EI index (enrichment index) of selected single
mutations in the first hybrid single domain.

| Position | Mutation | EI Index |
|---|---|---|
| L1 | H | 0.636477685 |
| L1 | W | 0.637400065 |
| Q2 | D | 0.917930978 |
| Q2 | T | 0.946831062 |
| E4 | A | 0.365806836 |
| E4 | W | 0.392513782 |
| I5 | V | 0.418917893 |
| F8 | Y | 0.452021407 |
| T9 | K | 0.424730805 |
| T9 | R | 0.533275349 |
| C10 | S | 0.35751446 |
| R11 | K | 0.608245997 |
| R11 | G | 0.645443413 |
| Q12 | F | 0.307819119 |
| Q12 | Y | 0.34576953 |
| K13 | Q | 0.278922437 |
| K13 | P | 0.280186349 |
| G14 | K | 0.306447148 |
| N15 | K | 0.205033159 |
| N15 | T | 0.298069121 |
| L16 | F | 0.516568047 |
| L16 | M | 0.552891676 |
| L17 | Y | 0.35840278 |
| L17 | Q | 0.457417453 |
| R18 | K | 0.285725983 |
| R18 | F | 0.361286698 |
| 20I | Y | 0.3727609 |
| 20I | R | 0.453876376 |
| K21 | S | 0.294182544 |
| K21 | Q | 0.300387833 |
| L22 | R | 0.372469467 |
| L22 | S | 0.401515932 |

Although in a preferred embodiment the degron tags and/or hybrid zinc finger polypeptides according to the invention comprise a first hybrid zinc finger domain comprising the sequence of any of those listed in Table 5, it is also contemplated the first hybrid zinc finger domain of the degron tags and/or hybrid zinc finger polypeptides according to the invention comprises either the beta hairpin of the first hybrid zinc finger domains listed in Table 5 or the alpha helix of the first hybrid zinc finger domains listed in Table 5, and further comprises a different alpha helix and beta hairpin portion respectively. For example, in a preferred embodiment, it is contemplated that the first hybrid zinc finger domain is comprised of a beta hairpin portion of any one of the sequences listed in Table 5 in combination with an alpha helix portion of any one of the sequences listed in Table 5. In some embodiments, the first hybrid zinc finger domain is comprised of a beta hairpin portion of any one of the sequences listed in Table 5 in combination with a further alpha helix portion not necessarily listed in Table 5. In some embodiments, the first hybrid zinc finger domain is comprised of a beta hairpin portion not necessarily listed in Table 5 in combination with an alpha helix portion of any one of the sequences listed in Table 5.

In some embodiments the first hybrid zinc finger domain of the degron tag and/or zinc finger polypeptide according to the invention is any one of those listed in Table 5, preferably any one SEQ ID 1-90, SEQ ID 1-80, SEQ ID 1-70, SEQ ID 1-60, SEQ ID 1-50, SEQ ID 1-40, SEQ ID 1-30, SEQ ID 1-20, SEQ ID 1-10, or SEQ ID NO: 135. In another embodiment the first hybrid zinc finger domain of the degron tag and/or zinc finger polypeptide according to the invention is any one of SEQ ID 1-10, SEQ ID 11-20, SEQ ID 21-30, SEQ ID 31-40, SEQ ID 41-50, SEQ ID 51-60, SEQ ID 61-70, SEQ ID 71-80, SEQ ID 81-90, or SEQ ID 91-100, or or SEQ ID NO: 135. In a further embodiment, the first hybrid zinc finger domain as shown in FIG. 3 (ZFP91 ZF4-IKZF1 ZF2) is replaced by any one of the sequences listed in Table 5.

Although for the current invention it is not necessary to include a second zinc finger domain, or to include a second hybrid zinc finger domain in the degron tags and/or zinc finger polypeptide according to the invention (as is discussed below), it was surprisingly found that sensitivity of the degron tags and/or zinc finger polypeptide according to the invention may be further enhanced or increased by combining the first hybrid zinc finger domain (e.g. having the at least two substitutions) with a further, second zinc finger domain, preferably a second hybrid zinc finger domain as described herein. Although various second zinc finger domains and/or second hybrid zinc finger domains may be used (as discussed herein elsewhere; for example those obtained from IKZF1 ZF3, IKZF3 ZF3, ZFP91 ZF5, ZNF653 ZF5, ZNF276 ZF5, ZNF827 ZF2, ZNF692 ZF5 (see also Uniprot Accession no: Q13422, Q9UKT9, Q96JP5, Q96CK0, Q8N554, Q17R98, Q9BU19, respectively), in preferred embodiments there is provided for a degron tag and/or hybrid zinc finger polypeptide according to the invention, and wherein the third portion of the second hybrid zinc finger domain comprises an amino acid sequence according to SEQ ID NO: 104 (FKCHLCNYACRR), the fourth portion of the second hybrid zinc finger domain comprises an amino acid sequence according to SEQ ID NO: 105 (KDSVVAHKAKSH), and/or wherein the second hybrid zinc finger comprises an amino acid sequence according to SEQ ID NO: 106 (FKCHLCNYACRRKDSVVAHKAKSH). In a preferred embodiment with respect to the second hybrid zinc finger, the second hybrid zinc finger is composed of a beta-hairpin portion of IKZF1 ZF3 and an alpha helix portion of ZFP91 ZF5.

According to another aspect of the invention, the first portion and/or the second portion of the first hybrid zinc finger domain are obtained from a first Cys2-His2 zinc finger domain and/or a second Cys₂-His₂ zinc finger domain that is a IMID substrate in the native protein having said first Cys2-His2 zinc finger domain and/or second Cys₂-His₂ zinc finger domain. Preferably the first portion and the second portion of the first hybrid zinc finger domain are obtained from a first Cys2-His2 zinc finger domain and a second Cys₂-His₂ zinc finger domain that both are IMID substrates in the native protein having said first Cys2-His2 zinc finger domain or second Cys₂-His₂ zinc finger domain.

According to another aspect of the invention, the first portion and/or the second portion of the first hybrid zinc finger domain are preferably obtained from a first Cys2-His2 zinc finger domain and/or a second Cys₂-His₂ zinc finger domain that is a IMiD substrate in the native protein having said first Cys2-His2 zinc finger domain and/or second Cys₂-His₂ zinc finger domain. Preferably the first portion and the second portion of the first hybrid zinc finger domain are obtained from a first Cys2-His2 zinc finger domain and a second Cys₂-His₂ zinc finger domain that both are IMiD substrates in the native protein having said first Cys2-His2 zinc finger domain or second Cys₂-His₂ zinc finger domain. According to the invention, and according to this aspect, in preferred embodiments, the third portion and/or the fourth portion of the second (hybrid) zinc finger domain (wherein “(hybrid) zinc finger domain” indicates herein either of both options, i.e. a second hybrid zinc finger domain or a second zinc finger domain) are a third Cys2-His2 zinc finger domain and/or a fourth Cys₂-His₂ zinc finger domain that is N-terminal or C-terminal of a Cys2-His2 zinc finger domain that is a IMiD substrate in the native protein, preferably C-terminal. In some embodiments, the third portion and/or the fourth portion are obtained from a Cys₂-His₂ zinc finger domain that is not an IMiD substrate in the native protein having said zinc finger domain.

Finally, and as already discussed herein in the context of a linker peptide between the first hybrid zinc finger domain and the second hybrid zinc finger domain, there is provided for a degron tag and/or hybrid zinc finger polypeptide according to the invention, and wherein the first hybrid zinc finger domain and the second zinc finger domain, preferably second hybrid zinc finger domain, are adjacent or are connected via a linker peptide, for example comprising one, two, three, four, five, six, seven or more amino acids. However, in some embodiments no additional linker peptide is present.

According to another aspect there is not only provided for a degron tag according to the invention, comprising a first hybrid zinc finger domain, further comprising at least two amino acid substitutions and comprising a second hybrid zinc finger domain. According to this aspect of the invention there is also provided for a non-natural hybrid zinc finger polypeptide comprising a first hybrid zinc finger domain comprising a first portion and a second portion wherein
(1) the first portion comprises the amino acid sequence X₁X₂C₃X₄X₅C₆X₇X₈X₉X₁₀X₁₁, wherein X represents any amino acid, of a first Cys₂-His₂ zinc finger domain;
(2) the second portion comprises the amino acid sequence X₁₂X₁₃X₁₄X₁₅X₁₆X₁₇X₁₈H₁₉X₂₀X₂₁X₂₂H₂₃, wherein X represents any amino acid, of a second Cys₂-His₂ zinc finger domain, wherein the second Cys₂-His₂ zinc finger domain is different from the first Cys₂-His₂ zinc finger domain;
(3) the second portion is C-terminal with respect to the first portion; and
(4) wherein the first and second portion together comprises (the) at least two amino acid substitutions, wherein an amino acid substitution in the first portion is relative to the first portion of the first Cys₂-His₂ zinc finger domain and wherein an amino acid substitution in the second portion is relative to the second portion of the second Cys₂-His₂ zinc finger domain, and wherein the substitution is not at any of positions C₃, C₆, X₇, H₁₉, or H₂₃.

Although experiments show that the presence of a second zinc finger domain or second hybrid zinc finger domain in the degron tag and/or zinc finger polypeptides according to the invention, experiments also make clear that such second zinc finger domain or second hybrid zinc finger domain is not necessary or required to provide for enhanced or increased sensitivity to IMiDs. In other words, it was also surprisingly established by the inventors there can be provided for increased or enhanced sensitivity to IMiDs by providing a hybrid zinc finger polypeptide as described herein. The hybrid zinc finger polypeptide comprise or consists of the first hybrid zinc finger domain that is already described herein in much detail, for example in the context of the degron tag according to the invention. It will thus be understood by the skilled person that all aspects, preferences and features (e.g., size, positions and type of substitutions (e.g., Tables 1-6), sequence, linkers and so on) detailed herein also apply to this aspect of the invention, and without the need to repeat these in full detail.

In a preferred embodiment of the invention the hybrid zinc finger polypeptide comprises any one of the sequences listed in Table 5. In some embodiments the hybrid zinc finger polypeptide according to the invention comprises any one of the sequences listed in Table 5, preferably any one SEQ ID 1-90, SEQ ID 1-80, SEQ ID 1-70, SEQ ID 1-60, SEQ ID 1-50, SEQ ID 1-40, SEQ ID 1-30, SEQ ID 1-20, SEQ ID 1-10, or SEQ ID NO: 135. In another embodiment the zinc finger polypeptide according to the invention comprises any one of SEQ ID 1-10, SEQ ID 11-20, SEQ ID 21-30, SEQ ID 31-40, SEQ ID 41-50, SEQ ID 51-60, SEQ ID 61-70, SEQ ID 71-80, SEQ ID 81-90, or SEQ ID 91-100 or SEQ ID NO: 135

In embodiments there is therefor provided for a non-natural hybrid zinc finger polypeptide according to the invention and wherein the non-natural hybrid zinc finger comprises a first hybrid zinc finger domain as defined herein.

Also provided is for a non-natural hybrid zinc finger polypeptide according to the invention further comprising a second zinc finger domain, wherein the second zinc finger domain is a non-hybrid zinc finger domain. As explained herein, although it may be beneficial to include a second zinc finger domain in the hybrid zinc finger polypeptide according to the invention, it has been established that such second zinc finger domain is not required to provide for the observed enhanced or increased sensitivity for IMiDs. Yet, in some embodiment, the non-natural hybrid zinc finger polypeptide according to the invention further comprising a second zinc finger domain, wherein the second zinc finger domain is a non-hybrid zinc finger domain. For example, the second zinc finger domain may be a wild type Cys₂-His₂ zinc finger domain, for example, such as those disclosed herein, for example IKZF1 ZF3, IKZF3 ZF3, but also ZFP91 ZF5, ZNF653 ZF5, ZNF276 ZF5, and/or ZNF827 ZF2, ZNF692 ZF5.

The second zinc finger domain is preferably oriented C-terminal relative to the first hybrid zinc finger domain and may be directly adjacent or may be linked to the first hybrid zinc finger domain, as described herein elsewhere.

In some embodiment, the second zinc finger domain is a second hybrid zinc finger domain, such as those described herein elsewhere. In those embodiments wherein the second zinc finger domain in the hybrid zinc finger domain according to the invention is a non-hybrid, e.g., wild type or naturally occurring zinc finger domain, or variation thereof, the second zinc finger domain may preferably be selected from e.g. IKZF1 ZF3 (SEQ ID NO: 109 (FKCHLCNYACRRRDALTGHLRTH) or IKZF3 ZF3 (SEQ ID NO: 110 (FKCHLCNYACQRRDALTGHLRTH) or comprises an amino acid sequence according to SEQ ID NO: 109 or SEQ ID NO: 110. Further examples of suitable second zinc finger domains include, for example, IKZF1 ZF3, IKZF3 ZF3, ZFP91 ZF5, ZNF653 ZF5, ZNF276 ZF5, ZNF827 ZF2, ZNF692 ZF5, SEQ ID NO: 109-115, and the like.

According to a further aspect there if provided for a fusion protein comprising a degron tag according to the invention, or a non-natural hybrid zinc finger polypeptide according to the invention, further comprising a protein of interest. This aspect of the invention is directed to a fusion protein including a protein of interest (POI) and a degron tag or hybrid zinc finger polypeptide according to the invention and that can mediate degradation of the POI comprising fusion protein in connection to the use of an IMiD.

In some embodiments, the degron tag of hybrid zinc finger polypeptide may be located N-terminal to the POI, C-terminal to the POI or within the POI.

The fusion protein according to the invention may be used in methods of degrading a protein of interest, and that includes contacting a cell expressing the fusion protein with an effective amount of an IMID. The protein of interest may be any suitable protein (of fragment thereof). In some embodiments, the protein of interest is an endogenous protein. In some embodiments, the protein of interest is an exogenous protein. In some embodiments, the protein of interest is a recombinant protein or non-natural protein. In some embodiments, the protein of interest is a natural occurring protein. Accordingly, the degron tags and/or hybrid zinc finger proteins of the current invention can be utilized to produce an endogenous protein-degron tag fusion protein or exogenous protein-degron tag fusion protein, for example stably expressed in a cell. Endogenous proteins originate within an organism, tissue or cell and is expressed by that same organism, tissue or cell, whereas exogenous proteins originate outside of an organism, tissue or cell and are introduced into the organism, tissue or cell.

Although the protein of interest in the fusion proteins according to the invention is not in particular limited to any particular type of protein, suitable example of protein of interest include, for example, Cbl-b, SOCS1, CISH, Tox, Eomes, IL12, IL15. CBL-B is an E3 ubiquitin-protein ligase that in humans is encoded by the CBLB gene. SOCS family proteins form part of a classical negative feedback system that regulates cytokine signal transduction. SOCS1 is involved in negative regulation of cytokines that signal through the JAK/STAT pathway. Through binding to JAKs and IFNGR1, inhibits their kinase activity. In vitro, also suppresses Tec protein-tyrosine activity. Cytokine-inducible SH2-containing (CISH) protein is a protein that in humans is encoded by the CISH gene CISH controls T cell receptor (TCR) signaling and contains a SH2 domain and a SOCS box domain. Thymocyte selection-associated high mobility group box protein TOX is a protein that in humans is encoded by the TOX gene. TOX drives T-cell exhaustion and plays a role in innate lymphoid cell development. Eomesodermin also known as T-box brain protein 2 (Tbr2) is a protein that in humans is encoded by the EOMES gene. Eomesodermin/Tbr2 is highly expressed in CD8+ T cells, but not CD4+ T cells. Interleukin 12 (IL-12) is an interleukin that is naturally produced by dendritic cells, macrophages, neutrophils, and human B-lymphoblastoid cells (NC-37) in response to antigenic stimulation. Interleukin-15 (IL-15) is a cytokine that binds to and signals through a complex composed of IL-2/IL-15 receptor beta chain (CD122) and the common gamma chain (gamma-C, CD132). IL-15 is secreted by mononuclear phagocytes (and some other cells) following infection by virus(es) and induces the proliferation of natural killer cells.

Other proteins of interest include, for example, such chimeric polypeptides as described in WO2021080427, allowing, for example, the time and/or dose dependent regulation of T cell activity as the consequence of signaling via T cell receptor (TCR) and/or chimeric antigen receptor (CAR) and for the time and/or dose dependent regulation of NK cell activity as the consequence of signaling via NK cell receptor (NKR) and/or chimeric antigen receptor (CAR). The chimeric polypeptide described therein is designed to interact with phosphorylated immunoreceptor tyrosine-based activation motifs (ITAM) in the TCR/CD3 complex and/or CAR and/or NK cell receptor (NKR) complexes that contain ITAM bearing signaling molecules such as DAP12, gamma chain of the immunoglobulin receptor FceRI or CD3 zeta chain (Lanier et al, Nat Immunol. 2008 May: 9 (5): 495-502).

The chimeric polypeptides of WO2021080427 provides for tight regulation of T cell and NK cell activity (e.g., cytotoxic activity and/or cytokine secretion) due to the presence of a small molecule-regulated protein stability domain that is utilized to modulate (e.g., reduce or increase) in a time and/or dose dependent manner the expression of the chimeric polypeptide. In preferred embodiment of the current invention, this/the small molecule-regulated protein stability domain is a degron tag and/or hybrid zinc finger polypeptide according to the invention. The protein of interest is as is disclosed in detail in WO2021080427, comprising a first part comprising a SH2-domain from a protein which binds to a phosphorylated immunoreceptor tyrosine-based activation motif (ITAM), and preferably also comprising a second part comprising an immunoreceptor tyrosine-based switch motif (ITSM), preferably an ITSM and an immunoreceptor tyrosine-based inhibitory motif (ITIM). This protein is preferably combined with a third part comprising a small molecule-regulated protein stability domain, which is, in accordance with the current invention a degron tag or a hybrid zinc finger polypeptide as disclosed herein. In some embodiments, the ITAM is an ITAM comprised in a T cell receptor (TCR) complex and/or a NK cell receptor (NKR) complex and/or a chimeric antigen receptor (CAR), preferably an ITAM derived from a CD3 zeta chain, a CD3 epsilon chain, a CD3 delta chain, CD3 gamma chain, gamma chain of the immunoglobulin receptor FceRI and DAP12. In some embodiments, the SH2-domain is from a protein selected from the group consisting of Zap70, Syk, and Lck. In some embodiments, the chimeric polypeptide comprises more than one SH2-domain from a protein which binds a phosphorylated immunoreceptor tyrosine-based activation motif. In some embodiments, the ITIM and/or ITSM is from an inhibitory receptor protein, preferably an inhibitory immune receptor protein, preferably from a protein selected from the group consisting of PD1, BTLA, SIRPalpha, SIGLEC5, SIGLEC9, SIGLEC11, PECAM1 or LY9.

Also provided is for a non-naturally occurring nucleic acid encoding a degron tag according to the invention, a non-natural hybrid zinc finger polypeptide according to the invention, or a fusion protein according to the invention.

Also provided is for a vector comprising such non-naturally occurring nucleic acid.

Also provided is for a cell or host cell which expresses the non-naturally occurring nucleic acid according to the invention. Preferably the cell, the host cell, the protein of interest, and/or the Cys₂-His₂ zinc finger domain or zinc finger domains, as well as other sequences in the degron tags and/or hybrid zinc finger polynucleotides according to the invention are human or are of human origin or consist essential of human sequences.

According to another aspect of the invention there is provided for a a method of providing a degron tag according to the invention, a non-natural hybrid zinc finger polypeptide according to the invention, a fusion protein according to the invention, or a nucleic acid encoding these, the method comprising
(A) providing a hybrid zinc finger domain comprising a first portion and a second portion wherein
(1) the first portion comprises the amino acid sequence X₁X₂C₃X₄X₅C₆X₇X₈X₉X₁₀X₁₁, wherein X represents any amino acid, of a first Cys₂-His₂ zinc finger domain;
(2) the second portion comprises the amino acid sequence X₁₂X₁₃X₁₄X₁₅X₁₆X₁₇X₁₈H₁₉X₂₀X₂₁X₂₂H₂₃, wherein X represents any amino acid, of a second Cys₂-His₂ zinc finger domain, wherein the second Cys₂-His₂ zinc finger domain is different from the first Cys₂-His₂ zinc finger domain;
(3) the second portion is C-terminal with respect to the first portion;
(B) introducing at least two different amino acid substitutions in the hybrid zinc finger domain, and wherein the substitutions are not at any of positions C₃, C₆, X₇, H₁₉, or H₂₃; and
(C) using the hybrid zinc finger domain obtained in step (B), or the nucleic acid sequence encoding the hybrid zinc finger domain obtained in step (B) in preparing a degron tag according to the invention, a non-natural hybrid zinc finger polypeptide according to the invention, a fusion protein according to any to invention, or a nucleic acid encoding these.

The skilled person is well aware of methods that allow for preparing a degron tag according to the invention, a non-natural hybrid zinc finger polypeptide according to the invention, a fusion protein according to the invention, or a nucleic acid encoding these. Such methods are readily available in the prior art and include those for example described in the examples.

The skilled person will also understand that with respect to the first portion, the second portion, as well as with respect to the first and second Cys₂-His₂ zinc finger domain, the same consideration, features, and preferences apply as those described herein elsewhere.

The skilled person will also understand that with respect to the at least two different amino acid substitutions that are introduced in the method of the invention, this may be at any position in the hybrid zinc finger domain comprising a first portion and a second portion, and the substitution may be into any amino acid (as long at the substituted amino acid is different from the substituting amino acid). The skilled person will also understand that in preferred embodiments the position, the substituted amino acid, and/or the substituting amino acid are those as described in relation to any one of Tables 1-6, and 8, including combinations of the at least two substitutions, as well as preferences indicated.

The method may include the step of testing the hybrid zinc finger domain comprising the at least two substitutions, and/or the degron tag according to the invention, a non-natural hybrid zinc finger polypeptide according to the invention, a fusion protein according to the invention, or a nucleic acid encoding these, obtained with the method of the invention of sensitivity for IMiDs, for example as described in the examples, for example by determining the EI value or index as described herein.

Based on the EI value or index it may be decided if the obtained hybrid zinc finger domain comprising the at least two substitutions, and/or the degron tag according to the invention, a non-natural hybrid zinc finger polypeptide according to the invention, a fusion protein according to the invention, or a nucleic acid encoding these should be discarded or not. Therefore, in a preferred embodiment the method includes a step of analyzing the obtained hybrid zinc finger domain comprising the at least two substitutions, and/or the degron tag according to the invention, a non-natural hybrid zinc finger polypeptide according to the invention, a fusion protein according to the invention, or a nucleic acid encoding these for sensitivity to an IMiD, for example as described in the example section, in order to determine its usefulness as a degron tag and the like.

Therefore, is an embodiment there is provided for establishing sensitivity of the degron tag according to the invention, a non-natural hybrid zinc finger polypeptide according to the invention, a fusion protein according to the invention, for example as obtained with the method according to the invention, for immunomodulatory imide drug (IMiD) induced degradation, preferably wherein the IMiD is selected from thalidomide, lenalidomide, pomalidomide, avadomide, iberdomide, salts and analogs thereof.

A degron tag according to the invention, a non-natural hybrid zinc finger polypeptide according to the invention, a fusion protein according to the invention may thus be selected for its ability to be induced by a particular small molecule, preferably an immunomodulatory inducing drug (IMID). In one aspect, the IMID is a thalidomide or one of its analogues, for example, lenalidomide, pomalidomide, avadomide, or iberdomide.

According to yet another aspect there is provided for a method of degrading a protein of interest or for controlling expression of a protein of interest comprising contacting a cell in vitro or in vivo with an effective amount of an IMiD, wherein the cell expresses a nucleic acid encoding a degron tag according to the invention, and/or a non-natural hybrid zinc finger polypeptide according to the invention, a fusion protein according to the invention, preferably wherein the method is for regulating the activity of the protein of interest.

According to a further aspect there is provided for a method of degrading a protein of interest or for controlling expression of a protein of interest comprising administering an effective amount of an IMiD to a subject, preferably a human subject, wherein the subject previously been treated via gene therapy causing at least some cells in the subject to express a nucleic acid encoding a degron tag according to the invention, a non-natural hybrid zinc finger polypeptide according to the invention, or a fusion protein according to the invention, preferably comprising the protein of interest. In a preferred embodiment, the method is a method wherein gene therapy comprises introducing cells to the subject (e.g., T cells, B cells or NK cells), wherein the introduced cells express a nucleic acid encoding a degron tag according to the invention, a non-natural hybrid zinc finger polypeptide according to the invention, or a fusion protein according to the invention, e.g., comprising the protein of interest.

These aspects of invention are directed to a method of degrading a protein of interest and that include, for example, contacting a transgenic cell with an effective amount of an IMID, wherein the cell, for example, produces a fusion protein including a protein of interest and at least one degron tag and/or hybrid zinc finger polynucleotide according to the invention.

The methods may be conducted in vivo or in vitro. The POIs may be exogenous or endogenous. The ability to degrade a particular endogenous or exogenous protein of interest by creating POI-degron tag fusions and administering an IMiD can be used to treat disorders by controlling cellular behavior, for example as described in WO2021080427.

Accordingly, the degron tags, hybrid zinc finger polynucleotides and fusion proteins of the present invention can be utilized to produce a stably expressed endogenous protein-degron tag fusion protein or exogenous protein-degron tag fusion protein, and which levels or cellular activity may be regulated by providing or removing IMiDs to or from cells expressing such fusion proteins.

Also provided is for the use of a nucleic acid encoding a degron tag according to the invention, a non-natural hybrid zinc finger polypeptide according to the invention, or a fusion protein according to the invention in the treatment of a subject, preferably a human subject. The treatment of the patient in need thereof may be as described herein or may be any other treatment.

Therefore, these is also provided for the use of a degron tag according to the invention, a non-natural hybrid zinc finger polypeptide according to the invention, a fusion protein according to the invention, or a nucleic acid encoding any of these in controlling transcription factors such as Eomes, Tox, negative regulators of TCR signaling, such as Cbl-b, negative regulators of cytokine receptors such as SOCS1, CISH, membrane bound cytokines such as IL12, IL15, antigen receptors such as TCR, CAR, NKR, checkpoint receptors such as PD1, LAG3, TIM3, nucleases such as Cas9, TALEN, or zinc finger nuclease.

Also provided for is a first hybrid zinc finger domain and/or a second hybrid zinc finger domain as disclosed herein.

Also provided is for the use of a degron tag according to the invention, a non-natural hybrid zinc finger polypeptide according to the invention, a fusion protein according to the invention, a first hybrid zinc finger domain according to the invention, a second hybrid zinc finger domain according to the invention or a nucleic acid encoding any of these, as a medicament, preferably in combination with the use of an IMID as a medicament. Pharmaceutical compositions comprising a degron tag according to the invention, a non-natural hybrid zinc finger polypeptide according to the invention, a fusion protein according to the invention, a first hybrid zinc finger domain according to the invention, a second hybrid zinc finger domain according to the invention or a nucleic acid encoding any of these are also provided, including those in combination with an IMiD.

In an aspect, a degron tag according to the invention, a non-natural hybrid zinc finger polypeptide according to the invention, or a fusion protein according to the invention has enhanced or increased sensitivity to an IMiD molecule, e.g., thalidomide analogue relative to, for example, wild-type zinc finger domains.

In an aspect, a degron tag according to the invention, a non-natural hybrid zinc finger polypeptide according to the invention, or a fusion protein according to the invention has enhanced or increased sensitivity to one or more IMiD molecules relative to the wild-type Cys₂-His₂ zinc finger domain from which the beta-hairpins and/or the alpha helix subdomains are derived.

In one embodiment, the enhanced or increased sensitivity to one or more IMiD molecules allows for a reduction in the amount of IMiD molecule administered to induce degradation by 5%, 10%, 15%, 20%, 25%, 30%, 35%, 40%, 45%, 50%, 55%, 60%, 65%, 70%, 75%, 80% or more. In an aspect, the amount of small molecule, e.g., IMiD molecule, administered is reduced by a factor of 2, 3, 4, 5, 6, 7, 8, 9, 10, 20, 50, 100, 110, 120, 130, 140, 150 or more.

The foregoing description of the specific embodiments will so fully reveal the general nature of the invention that others can, by applying knowledge within the skill of the art (including the contents of the references cited herein), readily modify and/or adapt for various applications such specific embodiments, without undue experimentation, without departing from the general concept of the present invention. Therefore, such adaptations and modifications are intended to be within the meaning and range of equivalents of the disclosed embodiments, based on the teaching and guidance presented herein.

All references cited herein, including journal articles or abstracts, published or corresponding patent applications, patents, or any other references, are entirely incorporated by reference herein, including all data, tables, figures, and text presented in the cited references. Additionally, the entire contents of the references cited within the references cited herein are also entirely incorporated by references.

It is to be understood that the phraseology or terminology herein is for the purpose of description and not of limitation, such that the terminology or phraseology of the present specification is to be interpreted by the skilled artisan in light of the teachings and guidance presented herein, in combination with the knowledge of one of ordinary skill in the art.

It will be understood that all details, embodiments and preferences discussed with respect to one aspect of embodiment of the invention is likewise applicable to any other aspect or embodiment of the invention and that there is therefore not need to detail all such details, embodiments and preferences for all aspect separately.

Having now generally described the invention, the same will be more readily understood through reference to the following examples which is provided by way of illustration and is not intended to be limiting of the present invention. Further aspects and embodiments will be apparent to those skilled in the art.

### EXAMPLES

Because of the concerns associated with IMiD use at higher doses, the identification of novel zinc finger degron tag sequences that can be regulated at lower drug doses is desired. In prior work (Sievers et al. Science. 2018 November 2; 362 (6414): eaat0572), hybrid zinc finger sequences were described that combine a beta-hairpin and alpha-helix from two different zinc finger degrons, and that display an increased sensitivity towards IMID-induced degradation, as compared to the parental zinc finger degron.

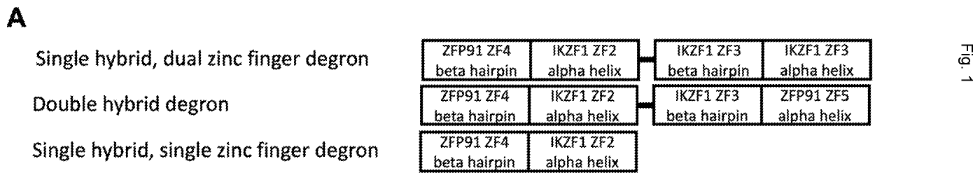

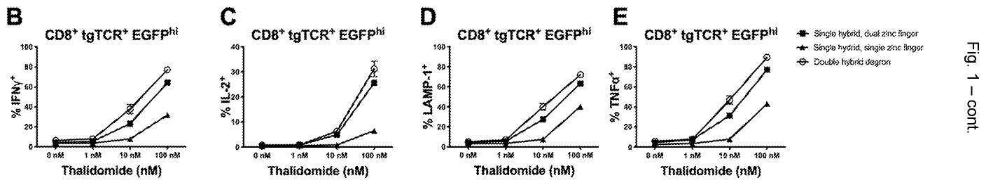

Specifically, a hybrid zinc finger degron containing the ZFP91 ZF4 beta hairpin and IKZF1 ZF2 alpha helix was found to show increase sensitivity towards IMiD induced degradation as compared to non-hybrid ZFP91 ZF4 and IKZF1 ZF2 zinc finger degrons. We previously created a ZFP91 ZF4 beta hairpin-IKZF1 ZF2 alpha helix-IKZF1 ZF3 degron (abbreviated as single hybrid, dual zinc finger prior art degron, FIG. 1) and demonstrated that CRASH-IT switch designs that contain this single hybrid, dual zinc finger prior art degron domain can be regulated by IMID molecules (WO2021080427A1). Here, we identify novel zinc finger-based degron domains that contain modifications in the first and, preferably the second zinc finger motifs of the degron tag and that show a significantly improved response to lower drug concentrations. Based on these data, we propose these novel degron domains, termed synthetic zinc fingers (SynFingers), as optimal building blocks to control the abundance of proteins of interest

### Example 1

### Material and Methods

### Cell Lines and Cell Culture

FLYRD18 (Sigma-Aldrich), NKIRTIL006 (Kvistborg et al. Oncoimmunology. 2012 Jul. 1; 1(4):409-418) and Jurkat cells were cultured in IMDM (ThermoFisher), supplemented with 8% FCS (ThermoFisher) and penicillin-streptomycin (100 IU/ml penicillin, 100 μg/ml streptomycin, Sigma-Aldrich). FLYRD18 and NKIRTIL006 cells were passaged every 2-3 days with trypsin-EDTA (ThermoFisher). All cell lines were tested for mycoplasma using PCR based screening and found negative.

### Retrovirus Production

Retroviral particles were produced in FLYRD18 packaging cells. In brief, 700,000 FLYRD18 packaging cells were plated per 10 cm dish one day prior to transfection. The next day, cell culture medium was refreshed with IMDM supplemented with 8% FCS without antibiotics. 25 μl of X-tremeGENE 9 (Roche) was mixed with 800 μl Opti-MEM (ThermoFisher) and incubated for 5 minutes. Subsequently, the Optimem-X-tremeGENE 9 mixture was added on top of 10 μg retroviral plasmid DNA dissolved in water and incubated for 15 minutes, and the resulting transfection mixture was added dropwise onto the packaging cells. Retrovirus containing supernatant was harvested 48 hours after transfection and immediately used or snap-frozen in liquid nitrogen.

### Plasmids

The retroviral vector encoding a CRASH-IT containing the single hybrid, dual zinc finger prior art degron, (pMP71-Zap70 2×SH2-PD1-ZFP91 ZF4 beta hairpin-IKZF1 ZF2 alpha helix-IKZF1 ZF3 iresEGFP, WO2021080427A1), a CRASH-IT variant containing a single hybrid, single zinc finger degron (pMP71-Zap70 2×SH2-PD1-(ZFP91 ZF4 beta hairpin-IKZF1 ZF2 alpha helix iresEGFP, WO2021080427A1), and the HLA class I-restricted CDK4 TCR (TCR 17, Stronen et al, Science. 2016 Jun. 10; 352 (6291): 1337-41) have been described previously.

The IKZF1 ZF3 alpha helix encoding sequence in the pMP71-Zap70 2×SH2-PD1-ZFP91 ZF4 beta hairpin-IKZF1 ZF2 alpha helix-IKZF1 ZF3 iresEGFP vector was replaced by gene synthesis product (IDT, IA, USA) encoding ZFP91 ZF5 alpha helix sequence using the Gibson assembly method (Gibson et al. Nat Methods. 2009 May; 6 (5): 343-5). The resulting degron is named “double hybrid degron” (FIG. 3).

A high diversity SynFinger library (˜50,200 variants) was synthesized by Twist Biosciences, CA, USA) using combinatorial variant library (CVL) service, as described in FIG. 3. pMP71-Zap70-Siglec11-FKBP12F36V iresEGFP has been described previously (Sahillioglu et al. Hum Gene Ther. 2021 October; 32 (19-20): 1029-1043). The FKBP12F36V encoding sequence in this vector was replaced by gene synthesis products encoding double hybrid degron (FIG. 3) or double hybrid degron variants containing the G14N/K21A, G14M/N15R, L17I/K21L, E4R/Q12L, Q12R/K1T or Q12R/K13V dual substitutions, or Q12R/K13V/G14N/K21A quadruple substitutions (SEQ ID NO: 135; SEQ ID NO: 136) using the Gibson assembly method.

### Screening

Quality control of the SynFinger library was performed by Twist Biosciences, and all but 2 SynFinger sequences amongst ˜50,200 synthesized sequences as described in FIG. 3 were observed in the cloned library. 150,000 million Jurkat cells were transduced with the SynFinger library (SynFinger containing CRASH-IT switches) at a 12.8% transduction efficiency and sorted for high EGFP expression to enrich transduced cells. For quality control of the SynFinger transduced Jurkat cells, SynFinger sequences were amplified and analyzed by NGS. 10 randomly selected SynFinger encoding DNA sequences or DNA sequences encoding the same SynFinger amino acid sequences but contain 2 nucleotide mismatches due to alternative codon usage were used to assess ability to distinguish true SynFinger sequences from noise in the NGS data. 10/10 SynFinger encoding full-nucleotide sequence match sequences were observed within NGS data whereas the 2 nucleotide mismatch containing sequences were not detected.

Jurkat cells expressing the SynFinger library were treated with 50 nM lenalidomide (a suboptimal dose that activates only ˜50% of cells expressing a CRASH-IT switch containing the parental double hybrid degron in Jurkat cells, as compared to a maximally effective 1000 nM lenalidomide dose) or were mock treated for 24 hours, and subsequently activated with anti-CD3/anti-CD28 antibodies in the presence or absence of 50 nM lenalidomide for 5 hours. Afterwards, cells were stained with IR dye (1:400) and anti-CD69-PE (1:200). Cells were sorted for live (IR dye negative), EGFP high end CD69 high (top 5%) or CD69 low (bottom 5%) expression using Aria Fusion cytometers.

Subsequently, cells were washed with PBS, and cell pellets were frozen at −20° C. until DNA extraction. Genomic DNA was isolated using the DNeasy Blood & Tissue Kit (Qiagen) according to the manufacturer's instructions, using 5×10\hat{ }6 cells per column. DNA was eluted in 22 μl RNAse free water. Deep sequencing adapters and indexes were added to SynFinger encoding DNA sequences using PCR amplification. Briefly, 3 μg genomic DNA (20 μl), indexed forward and reverse primers (10 μM, 2.5 μl each) and 25 μl NEBNext® High-Fidelity 2×PCR Master Mix were added to the PCR reaction. An optimized PCR reaction (98° C. for 30 s, (98° C. for 10 s, 56° C. for 10 s, 72° C. for 5 s)×25 cycles, 72° C. for 3 min was performed using Eppendorf PCR cyclers.

Indexed PCR products were run on agarose gel, relative DNA concentrations were estimated by measuring band intensity, and 12 indexed PCR reaction products were mixed per MiSeq chip. To increase sequence diversity, samples were mixed with 25% PhiX DNA. In total, 2 MiSeq chips were used (100 bp, single read). To distinguish genuine SynFinger sequences from sequencing errors, results were filtered using a reference list of actual sequences used in SynFinger library design (100% codon match). Sequences with a very low number of reads (sum of all samples below 500 reads) were excluded from analysis.

To identify SynFingers that improve sensitivity of CRASH-IT switches towards lenalidomide while preserving or improving stringency of switch mediated control of T cell activation in the absence of lenalidomide, an enrichment index (EI) value was calculated for each sequence as described below, and SynFingers were ranked according to their EI, with a low EI indicating a SynFinger with desired properties.

$$EI = \frac{Readcount ( Lenalidomide treatment . CD 69 low )}{Readcount ( Lenalidomide treatment . CD 69 high )} \times \frac{Readcount ( mock treatment . CD 69 high )}{Readcount ( mock treatment . CD 69 low )}$$

CD69-HI and CD60-LO read counts from top 100 SynFingers were compared with read counts from the parental double hybrid degron using a two-sided Fisher's exact test to determine enrichment of CD69-HI cells in the presence of lenalidomide and enrichment of CD69-LO cells in the absence of lenalidomide. The results for SEQ ID NO: 1-100 are shown in Table 6 and 9.

TABLE 9

| | Mock treatment | Lenalidomide | |
|---|---|---|---|
| | Fisher's exact | treatment | |
| SEQ ID | test | Fisher's exact test | |
| No. | p-value | p-value | EI index |
| 1 | 0.028722882 | 3.87E−115 | 0 |
| 2 | 3.40E−05 | 2.90E−130 | 0.00291222 |
| 3 | 4.41E−13 | 5.57E−89 | 0.00372528 |
| 4 | 1.34E−37 | 5.08E−202 | 0.004109971 |
| 5 | 0.412432478 | 9.93E−124 | 0.004648074 |
| 6 | 1.09E−53 | 1.46E−101 | 0.00471865 |
| 7 | 2.28E−26 | 2.08E−83 | 0.004968515 |
| 8 | 1.06E−32 | 1.31E−57 | 0.006186869 |
| 9 | 7.67E−05 | 9.75E−122 | 0.006877267 |
| 10 | 4.01E−20 | 1.12E−30 | 0.006950013 |
| 11 | 0.011641544 | 5.81E−73 | 0.008068234 |
| 12 | 6.50E−05 | 3.38E−77 | 0.0085639 |
| 13 | 8.56E−30 | 5.49E−61 | 0.00876145 |
| 14 | 1.84E−36 | 1.17E−175 | 0.009984402 |
| 15 | 0.000149089 | 8.41E−119 | 0.010142772 |
| 16 | 0.00016446 | 1.42E−115 | 0.010704273 |
| 17 | 0.000180527 | 4.40E−80 | 0.010796113 |
| 18 | 0.588330181 | 8.24E−165 | 0.011326673 |
| 19 | 2.77E−36 | 1.13E−17 | 0.011767926 |
| 20 | 1.21E−24 | 4.87E−17 | 0.012162914 |
| 21 | 7.82E−35 | 4.35E−12 | 0.012551551 |
| 22 | 1.13E−36 | 3.29E−15 | 0.013111269 |
| 23 | 2.70E−234 | 3.80E−153 | 0.013300837 |
| 24 | 1.29E−09 | 4.88E−29 | 0.013512849 |
| 25 | 7.64E−09 | 2.25E−119 | 0.013833226 |
| 26 | 0.177784834 | 4.69E−180 | 0.014775966 |
| 27 | 3.77E−26 | 5.36E−46 | 0.014865307 |
| 28 | 4.43E−47 | 2.28E−60 | 0.015890989 |
| 29 | 0.266451239 | 1.04E−69 | 0.016086794 |
| 30 | 2.90E−25 | 6.79E−51 | 0.016725195 |
| 31 | 0.074811465 | 7.73E−59 | 0.016863083 |
| 32 | 2.45E−14 | 1.79E−57 | 0.016927083 |
| 33 | 3.86E−73 | 4.23E−15 | 0.017054368 |
| 34 | 4.49E−29 | 1.35E−137 | 0.017225632 |
| 35 | 5.58E−52 | 1.23E−162 | 0.017544896 |
| 36 | 1.66E−49 | 3.45845952088873e−323 | 0.017694595 |
| 37 | 1.41E−44 | 1.14E−21 | 0.017868996 |
| 38 | 2.45E−38 | 2.83E−54 | 0.017942936 |
| 39 | 1.41E−07 | 8.55E−91 | 0.01900977 |
| 40 | 3.74E−42 | 0.014383253 | 0.01915403 |
| 41 | 2.45E−47 | 2.04E−30 | 0.019438445 |
| 42 | 3.17E−17 | 2.11E−117 | 0.019646848 |
| 43 | 2.22E−11 | 9.04E−63 | 0.019766277 |
| 44 | 0.936633417 | 2.00E−87 | 0.020643322 |
| 45 | 7.09E−67 | 0.004880501 | 0.020901639 |
| 46 | 7.01E−40 | 2.88E−07 | 0.020996596 |
| 47 | 0.367517193 | 1.50E−124 | 0.022086922 |
| 48 | 1.96E−35 | 1.03E−29 | 0.022190694 |
| 49 | 1.62E−07 | 1.17E−90 | 0.022681348 |
| 50 | 2.52E−48 | 2.56E−81 | 0.023070252 |
| 51 | 4.13E−08 | 1.01E−102 | 0.023086608 |
| 52 | 9.78E−72 | 0.374575991 | 0.023099481 |
| 53 | 2.13E−17 | 1.52E−99 | 0.023631198 |
| 54 | 0.067264505 | 3.23E−55 | 0.024078298 |
| 55 | 0.024132382 | 3.23E−45 | 0.024697019 |
| 56 | 0.36170993 | 3.06E−103 | 0.025430522 |
| 57 | 1.40E−75 | 2.38E−19 | 0.02583464 |
| 58 | 0.12435195 | 0 | 0.025841131 |
| 59 | 4.71E−06 | 5.86E−39 | 0.026815642 |
| 60 | 5.58E−51 | 9.17E−10 | 0.027389937 |
| 61 | 1.38E−60 | 0.011760083 | 0.027600513 |
| 62 | 3.74E−35 | 1.38E−26 | 0.027669711 |
| 63 | 3.22E−09 | 5.34391283854809e−319 | 0.028290744 |
| 64 | 1.17E−08 | 4.67E−28 | 0.029469901 |
| 65 | 0.006273013 | 6.23E−101 | 0.029885865 |
| 66 | 0.024578514 | 6.86E−98 | 0.029894605 |
| 67 | 5.29E−30 | 3.56E−21 | 0.030300224 |
| 68 | 0.096623275 | 5.46E−82 | 0.031406463 |
| 69 | 1.51E−32 | 1.22E−26 | 0.031878391 |
| 70 | 0.000309359 | 8.42E−97 | 0.031924717 |
| 71 | 2.24E−56 | 1.11E−13 | 0.032269961 |
| 72 | 2.24E−26 | 2.48E−52 | 0.032746025 |
| 73 | 3.27E−25 | 1.10E−27 | 0.033034775 |
| 74 | 0.082878836 | 8.46E−98 | 0.033618582 |
| 75 | 6.58E−08 | 3.38E−71 | 0.03442029 |
| 76 | 0.001747814 | 5.31E−81 | 0.034449761 |
| 77 | 8.43E−75 | 2.09E−16 | 0.035310273 |
| 78 | 5.48E−16 | 3.27E−32 | 0.035438461 |
| 79 | 0.624640971 | 8.36E−89 | 0.035691832 |
| 80 | 1.14E−17 | 1.28E−20 | 0.036848485 |
| 81 | 0 | 1.84E−10 | 0.037261051 |
| 82 | 6.85E−14 | 1.87E−58 | 0.037538608 |
| 83 | 9.64E−63 | 7.59E−199 | 0.038387133 |
| 84 | 6.93E−32 | 6.02E−38 | 0.039025335 |
| 85 | 8.57E−22 | 5.44E−49 | 0.039238539 |
| 86 | 2.24E−05 | 1.45E−30 | 0.039295393 |
| 87 | 1.25E−21 | 1.06E−50 | 0.039396688 |
| 88 | 1.87E−15 | 1.32E−21 | 0.039473684 |
| 89 | 9.73E−05 | 5.56E−53 | 0.040650091 |
| 90 | 9.52E−08 | 7.92E−30 | 0.0410691 |
| 91 | 8.05E−36 | 4.62E−09 | 0.04124576 |
| 92 | 1.12E−09 | 1.12E−37 | 0.041287832 |
| 93 | 0.001568011 | 6.53E−121 | 0.04175191 |
| 94 | 0.163528995 | 7.32E−57 | 0.042291085 |
| 95 | 1.06E−07 | 2.88E−78 | 0.042847145 |
| 96 | 6.17E−11 | 2.93E−22 | 0.042849089 |
| 97 | 3.41E−51 | 1.92E−29 | 0.042938713 |
| 98 | 1.13E−07 | 0 | 0.043024631 |
| 99 | 0.005332121 | 1.08E−35 | 0.043386844 |
| 100 | 0.884502214 | 2.53E−166 | 0.044608879 |

### Example 2

### Results

To our knowledge, the possibility of using a hybrid zinc finger as the second zinc finger as a means to improve the overall sensitivity of the dual zinc finger degron system was not tested before. To investigate whether the second zinc finger (IKZF1 ZF3) of the single hybrid, dual zinc finger prior art degron can be replaced with other zinc finger sequences while preserving or improving sensitivity towards IMiDs, we created a panel of CRASH-IT switches containing the zinc finger degrons shown in FIG. 1. Primary human T cells were transduced to express the CDK4 neoantigen-specific MHC class I-restricted TCR, together with CRASH-IT variants encoding indicated zinc finger degrons. Analysis of cytokine production (IFNγ, IL2, and TNFα) and T cell degranulation (LAMP-1 surface expression) upon co-culture with endogenously CDK4 neoantigen expressing NKIRTIL006 cells revealed that a double hybrid degron improved the sensitivity of the CRASH-IT switch towards thalidomide as compared to the CRASH-IT variant that contains the single hybrid, dual zinc finger prior art degron sequence (FIG. 1).

In line with prior data, a CRASH-IT switch containing a single hybrid, single zinc finger degron showed reduced thalidomide sensitivity as compared to the single hybrid, dual zinc finger prior art degron sequence (FIG. 1).

To further improve the sensitivity of double hybrid degron towards IMiDs, we performed a high throughput screen using a library of ˜50,200 CRASH-IT variants that encode either single or double amino acid substitutions at the indicated positions of the first hybrid zinc finger sequence, thereby changing 18 amino acids of this zinc finger into any amino acid except cysteine (FIG. 3). Jurkat T cells expressing this synthetic zinc finger (SynFinger) containing CRASH-IT variant library were treated with a low dose (50 nM) lenalidomide or left untreated for 24 hours, followed by stimulation with aCD3/aCD28 antibodies for 5 hours in the continued presence or absence of lenalidomide. Subsequently, 5% of Jurkat cells with the highest or lowest expression of the T cell activation marker CD69 were isolated by FACS, SynFinger sequences present in these populations were amplified and analyzed by NGS. Analysis of the relative abundance of SynFinger sequences in the CD69-HI and CD69-LO populations identified amino acid substitutions within the zinc finger degron that improved sensitivity towards IMiDs. Enrichment index (EI) values that both reflect high expression of CD69 in the presence of lenalidomide and low expression of CD69 in the absence of lenalidomide were calculated for each SynFinger (see Methods). SynFingers were then ranked according to EI values to identify novel zinc finger degrons with desirable characteristics, such as improved sensitivity towards IMiDs and efficient switch function (and hence stable POI expression) in the absence of IMID.

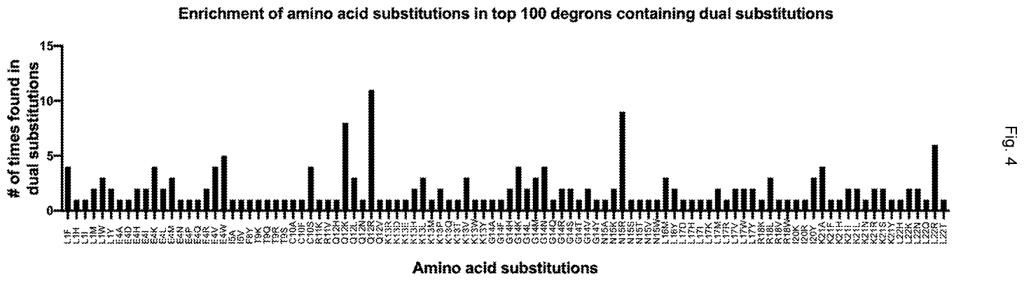

The top 100 SynFingers identified with this method were all dual amino acid substitution (Table 6). Importantly, certain amino acid substitutions such as Q12R, Q12K, N15R and L22R (present in 11%, 8%, 9% and 6% of top 100 SynFingers, respectively) were observed frequently among the top 100 SynFinger containing dual substitution mutations (FIG. 4). Notably, these mutations were also ranked among the top hits when analyzed as single amino acid substitutions. Furthermore, the frequently observed primary amino acid substitutions Q12R, Q12K, N15R and L22R, increased sensitivity towards IMiDs further in a synergistic manner when combined with the secondary amino acid substitutions listed in Table 8, (i.e., EI Q12R/K13V=0.006877267, whereas EI Q12R=0.167217557). Thus in particular preferred embodiments of the invention, a combination of amino acid substitutions are mentioned in table 8 is used/provided, e.g. 12R and 13T or 22R and 14L, and so on.

TABLE 8
Secondary mutations observed in combination with Q12R, N15R,
Q12K and L22R primary mutations in top 100 list of Table 5.

| Secondary | Secondary | Secondary | Secondary |
|---|---|---|---|
| mutations | mutations | mutations | mutations |
| observed in | observed in | observed in | observed in |
| combination | combination | combination | combination |
| with Q12R | with N15R | with Q12K | with L22R |
| K13T | G14M | N15A | G14N |
| L17M | G14T | F8Y | N15T |
| K13V | G14T | I5V | K13P |
| E4L | L1F | I20K | L17D |
| L17Y | L1H | G14N | G14L |
| L22H | K13D | K13R | T9K |
| C10A | G14Q | K21I | |
| I20R | C10S | G14V | |
| N15S | K21H | | |
| E4W | | | |
| E4Q | | | |

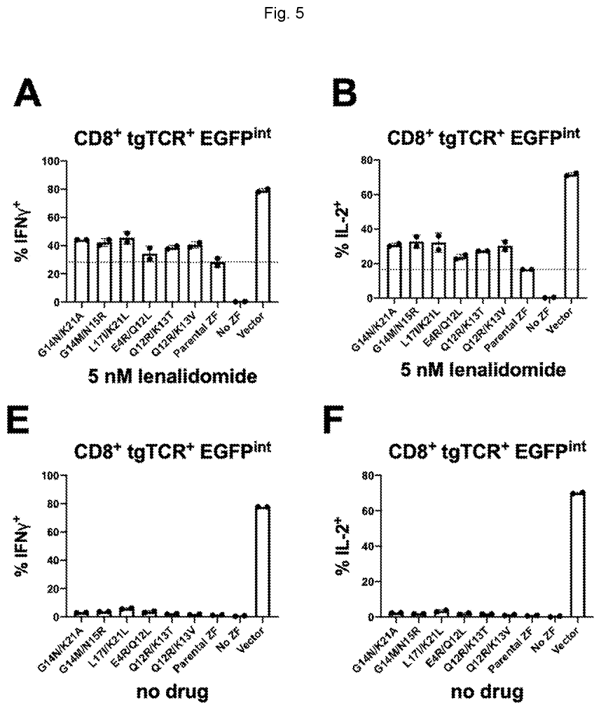

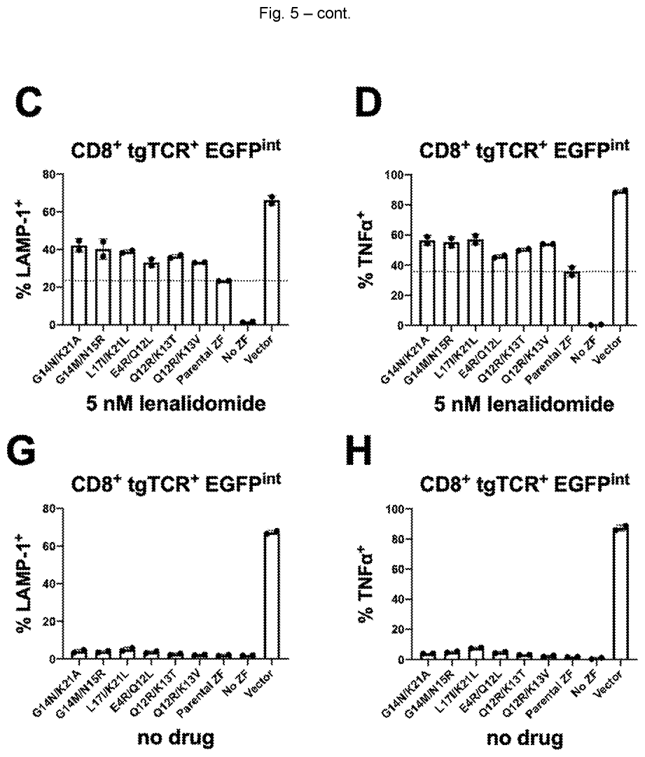

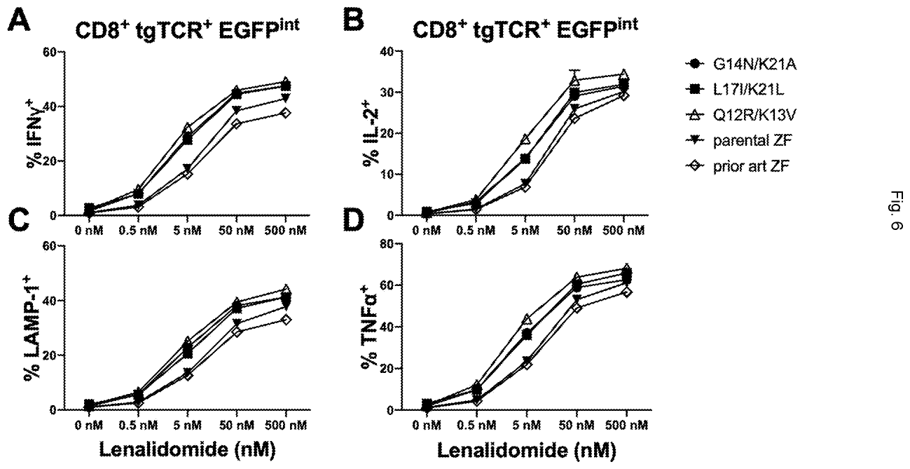

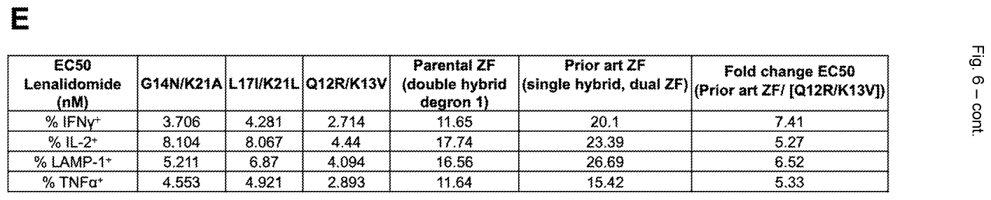

To test whether the SynFingers that show an improved drug sensitivity in this Jurkat SynFinger library screen also led to an improved sensitivity to low IMID concentrations when expressed in primary human T cells, human peripheral blood T cells were modified to express the CDK4 TCR and indicated SynFinger containing CRASH-IT switch variants (FIG. 5). Importantly, T cells expressing G14N/K21A, G14M/N15R, L17I/K21L, E4R/Q12L, Q12R/K1T and Q12R/K13V dual substitution containing SynFingers showed improved cytokine production at very low (5 nM) lenalidomide concentration levels as compared to T cells expressing the double hybrid degron containing CRASH-IT switch that formed the starting point of the genetic screen (FIG. 5). Furthermore, small molecule titration experiments revealed that the Q12R/K13V dual substitution containing SynFinger shows a 5.3- to 7.4-fold lower EC50 value as compared to the single hybrid, dual zinc finger prior art degron containing CRASH-IT switch for all T cell effector functions tested (FIG. 6).

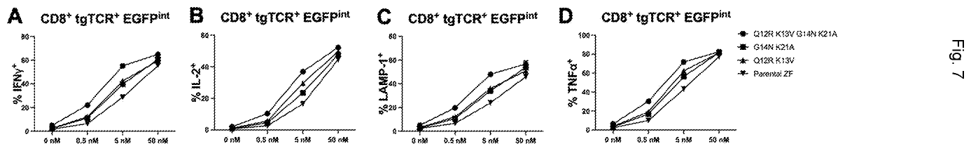

Upon identification of sets of 2 amino acid substitutions such as Q12R/K13V and G14N/K21A that improve lenalidomide sensitivity of SynFinger degrons, we hypothesized that combination of these sets could increase drug sensitivity further. To test this, we created CRASH-IT switch variants containing parental ZF, or SynFinger degrons with dual substitutions (Q12R/K13V or G14N/K21A) or quadruple substitutions (Q12R/K13V/G14N/K21A; SEQ ID NO: 135 and SEQ ID NO: 136). Primary human T cells were transduced to express the CDK4 TCR, together with indicated CRASH-IT variants. Analysis of cytokine production (IFNγ, IL2, and TNFα) and T cell degranulation (LAMP-1 surface expression) upon co-culture with endogenously CDK4 neoantigen expressing NKIRTIL006 cells revealed that combination of Q12R/K13V/G14N/K21A substitutions improved the sensitivity of the SynFinger towards lenalidomide as compared to the SynFinger degrons that contain Q12R/K13V or G14N/K21A (FIG. 7).

Thus, these data identify specific positions and mutations in zinc finger sequences that yield improved substrates for regulation by IMiDs and also specific combinations of amino acid mutations that yield such improved substrates. Note that in the SynFinger validation experiments (FIG. 5 and FIG. 6), Siglec11 signaling domain containing CRASH-IT variants were used, whereas in the high throughout SynFinger library screening (FIG. 3 and FIG. 4), the CRASH-IT variant contained the PD1 signaling domain, demonstrating that optimized SynFingers improve control over cell function independent of the POI effector domain used.

The synthetic zinc finger (SynFinger) sequences we identified in this study may be used in a range of applications beyond their use in the CRASH-IT switch platform. For example, important regulators of cell function, such as receptors that determine the antigen specificity of immune cells, but also regulators controlling antigen sensitivity, cytokine signaling, or cell differentiation may be regulated by creating fusion proteins with such novel SynFinger sequences. The ability to induce protein degradation at reduced IMiD levels opens new possibilities to implement this protein degradation technology in clinical settings both in cellular therapies and beyond.

1. A degron tag comprising a non-natural first hybrid zinc finger domain and a non-natural second hybrid zinc finger domain wherein
(1) the first hybrid zinc finger domain comprises a first portion and a second portion;
(2) the first portion comprises the amino acid sequence X₁X₂C₃X₄X₅C₆X₇X₈X₉X₁₀X₁₁, wherein X represents any amino acid, of a first Cys₂-His₂ zinc finger domain;
(3) the second portion comprises the amino acid sequence X₁₂X₁₃X₁₄X₁₅X₁₆X₁₇X₁₈H₁₉X₂₀X₂₁X₂₂H₂₃, wherein X represents any amino acid, of a second Cys₂-His₂ zinc finger domain, wherein the second Cys₂-His₂ zinc finger domain is different from the first Cys₂-His₂ zinc finger domain;
(4) the second portion is C-terminal with respect to the first portion;
(5) the second hybrid zinc finger domain is C-terminal with respect to the first hybrid zinc finger domain;
(6) the second hybrid zinc finger domain comprises a third portion and a fourth portion;
(7) the third portion comprises the amino acid sequence (Z)₂C(Z)₂C(Z)₅₋₆ wherein Z represents any amino acid, of a third Cys₂-His₂ zinc finger domain;
(8) the fourth second portion comprises the amino acid sequence (Z)₆H(Z)₃₋₄H, wherein Z represents any amino acid, of a fourth Cys₂-His₂ zinc finger domain, wherein the fourth Cys₂-His₂ zinc finger domain is different from the third Cys₂-His₂ zinc finger domain; and
(9) the fourth portion is C-terminal with respect to the third portion; and
(10) wherein the first hybrid zinc finger domain comprises at least two amino acid substitutions, wherein an amino acid substitution in the first portion of the first hybrid zinc finger domain is relative to the first portion of the first Cys₂-His₂ zinc finger domain and wherein an amino acid substitution in the second portion of the first hybrid zinc finger domain is relative to the second portion of the second Cys₂-His₂ zinc finger domain, and wherein the substitution is not at any of positions C₃, C₆, X₇, H₁₉, or H₂₃.

2. The degron tag of claim 1, wherein the degron tag is able to bind a complex formed between cereblon (CRBN) and an immunomodulatory drug (IMiD).

3. The degron tag of claim 1, wherein the first portion is a beta-hairpin loop of a first Cys₂-His₂ zinc finger domain, the second portion is an alpha-helix region of a second Cys₂-His₂ zinc finger domain, the third portion is a beta-hairpin loop of a third Cys₂-His₂ zinc finger domain, and/or the fourth portion is an alpha-helix region of a fourth Cys₂-His₂ zinc finger domain.

4. The degron tag of claim 1, wherein the first substitution is in the second portion and the second substitution is in the first portion or in the second portion.

5. The degron tag of claim 1, wherein at least one substitution is at a position selected from the group consisting of X₁, X₄, X₁₂, X₁₃, X₁₄, X₁₅, X₁₇, X₂₁, and X₂₂.

6. The degron tag of claim 1, wherein at least one substitution is selected from those listed in Table 1, preferably wherein the at least two substitutions are each, independently, selected from those listed in Table 1.

7. The degron tag of claim 1, wherein the at least two substitutions are selected from those listed in Table 2.

8. The degron tag of claim 1, wherein an amino acid in the first portion that is substituted and/or an amino acid in the second portion that is substituted is selected from those listed in Table 3.

9. The degron tag of claim 1, wherein the at least two amino acids in the first portion and/or in the second portion that are substituted are selected from those listed in Table 4.

10. The degron tag of claim 1, wherein:
(1) the first Cys₂-His₂ zinc finger domain is selected from the group consisting of IKZF1 ZF2, IKZF3 ZF2, ZFP91 ZF4, ZNF654 ZF1, ZNF787 ZF5, ZNF653 ZF4, ZNF276 ZF4, ZNF692 ZF4, ZNF582 ZF9, ZNF517 ZF10, E4F1 ZF2, and ZNF827 ZF1, preferably ZFP91 ZF4;
(2) the second Cys₂-His₂ zinc finger domain is selected from the group consisting of IKZF1 ZF2, IKZF3 ZF2, ZFP91 ZF4, ZNF654 ZF1, ZNF787 ZF5, ZNF653 ZF4, ZNF276 ZF4, ZNF692 ZF4, ZNF582 ZF9, ZNF517 ZF10, E4F1 ZF2, ZNF827 ZF1, preferably IKZF1 ZF2;
(3) the third Cys₂-His₂ zinc finger domain is selected from the group consisting of IKZF1 ZF3, IKZF3 ZF3, ZFP91 ZF5, ZNF653 ZF5, ZNF276 ZF5, ZNF827 ZF2, ZNF692 ZF5, preferably IKZF1 ZF3; and/or
(4) the fourth Cys₂-His₂ zinc finger domain is selected from the group consisting of IKZF1 ZF3, IKZF3 ZF3, ZFP91 ZF5, ZNF653 ZF5, ZNF276 ZF5, ZNF827 ZF2, ZNF692 ZF5, preferably ZFP91 ZF5.

11. The degron tag of claim 1, wherein the at least two amino acid substitutions are introduced in a first hybrid zinc finger domain the first portion thereof having an amino acid sequence according to SEQ ID NO: 101, and/or the second portion thereof having an amino acid sequence according to SEQ ID NO: 102, and/or the first hybrid zinc finger domain having an amino acid sequence according to SEQ ID NO: 103.

12. The degron tag of claim 1, wherein the first hybrid zinc finger domain comprising the at least two substitutions is selected from those listed in Table 5.

13. The degron tag of claim 1, wherein the third portion of the second hybrid zinc finger domain comprises an amino acid sequence according to SEQ ID NO: 104, the fourth portion of the second hybrid zinc finger domain comprises an amino acid sequence according to SEQ ID NO: 105, and/or wherein the second hybrid zinc finger comprises an amino acid sequence according to SEQ ID NO: 106.

14. The degron tag of claim 1, wherein the first hybrid zinc finger domain and the second hybrid zinc finger domain are adjacent or are connected via a linker peptide.

15. A non-natural hybrid zinc finger polypeptide comprising a first hybrid zinc finger domain comprising a first portion and a second portion wherein
(1) the first portion comprises the amino acid sequence X₁X₂C₃X₄X₅C₆X₇X₈X₉X₁₀X₁₁, wherein X represents any amino acid, of a first Cys₂-His₂ zinc finger domain;
(2) the second portion comprises the amino acid sequence X₁₂X₁₃X₁₄X₁₅X₁₆X₁₇X₁₈H₁₉X₂₀X₂₁X₂₂H₂₃, wherein X represents any amino acid, of a second Cys₂-His₂ zinc finger domain, wherein the second Cys₂-His₂ zinc finger domain is different from the first Cys₂-His₂ zinc finger domain;
(3) the second portion is C-terminal with respect to the first portion; and
(4) wherein the first and second portion together comprises at least two amino acid substitutions, wherein an amino acid substitution in the first portion is relative to the first portion of the first Cys₂-His₂ zinc finger domain and wherein an amino acid substitution in the second portion is relative to the second portion of the second Cys₂-His₂ zinc finger domain, and wherein the substitution is not at any of positions C₃, C₆, X₇, H₁₉, or H₂₃.

16. (canceled)

17. The non-natural hybrid zinc finger polypeptide of claim 15, further comprising a second zinc finger domain, wherein the second zinc finger domain is a non-hybrid zinc finger domain, preferably selected from IKZF1 ZF3, IKZF3 ZF3, but also ZFP91 ZF5, ZNF653 ZF5, ZNF276 ZF5, and/or ZNF827 ZF2, ZNF692 ZF5.

18. The non-natural hybrid zinc finger polypeptide of claim 15, wherein the second zinc finger domain is C-terminal with respect to the first hybrid zinc finger domain.

19. The non-natural hybrid zinc finger polypeptide of claim 15, wherein the second zinc finger domain is selected from IKZF1 ZF3, IKZF3 ZF3 and/or wherein the second zinc finger domain comprises an amino acid sequence according to SEQ ID NO: 109-110.

20. A fusion protein comprising a degron tag of claim 19 and a protein of interest.

21. The fusion protein of claim 20, wherein the degron tag is located N-terminal or C-terminal to the protein of interest.

22. The fusion protein of any of the previous claims, wherein the protein of interest is selected from Cbl-b, SOCS1, CISH, Tox, Eomes, IL12, IL15.

23. (canceled)

24. (canceled)

25. (canceled)

26. (canceled)

27. (canceled)

28. (canceled)

29. (canceled)

30. (canceled)

31. (canceled)

32. (canceled)

33. A fusion protein comprising a non-natural hybrid zinc finger polypeptide of of claim 16 and a protein of interest.

34. The fusion protein of claim 33, wherein the non-natural hybrid zinc finger polypeptide is located N-terminal or C-terminal to the protein of interest.

35. The fusion protein of any of the previous claims, wherein the protein of interest is selected from Chl-b, SOCS1, CISH, Tox, Eomes, IL12, IL15.
